(19) United States
(12) Patent Application Publication
(10) Pub. No.: US 2026/0273083 A1
(43) Pub. Date: Sep. 17, 2026

(54) Anti-human CD98hc antibody and methods of use therefor

(71) Applicant: MedAbome, Inc., Fremont, CA (US)

(72) Inventors: Mason Lu, Fremont, CA (US); Qinhong Ma, Fremont, CA (US); Mary Q. Xu, Houston, TX (US); Daizong Li, Clayton, MO (US); Ke-Wei Zhao, El Monte, CA (US)

(21) Appl. No.: 19/077,109

(22) Filed: Mar. 12, 2025

(51) Int. Cl. A61K 47/68; C07K 16/28

(52) U.S. Cl. CPC A61K 47/68031; A61K 47/6849; A61K 47/6889; C07K 16/2896; C07K 2317/31; C07K 2317/565; C07K 2317/622; C07K 2317/77

(57) ABSTRACT

Disclosed is a monoclonal antibody, MAb52-4.2 mAb, and its fragments and derivatives that recognize a conformational epitope of human CD98hc (SLC3A2, 4F2HC), which is present highly on malignant tumors but weakly or is absent in normal human cells and tissues. This specificity enhances its potential as a therapeutic agent while minimizing off-target effects. The anti-CD98 hc mAb and its fragments and derivatives can be conjugated to cytotoxic drug moieties to form antibody-drug conjugates (ADCs), to inhibit tumor growth in animal models.

### FIELD OF THE INVENTION

The present invention relates to a monoclonal antibody, MAb52-4.2 mAb, and its derivatives that recognize a conformational epitope of human CD98hc, which is highly expressed on many solid tumors and hematologic malignancies, but rarely found in normal tissues, thereby enhancing its potential as a therapeutic agent while minimizing off-target effects. MAb52-4.2 mAb and its derivatives can be conjugated to cytotoxic drug moieties to form antibody-drug conjugates (ADCs) to inhibit solid tumor growth in animal models.

### BACKGROUND

CD98 heavy chain (CD98hc), also known as SLC3A2 (solute carrier family 3 member 2), 4F2, MDU1, 4F2HC, 4T2HC, and NACAE, is a type II membrane protein encoded by the SLC3A2 gene. It has long been implicated in various physiological and pathological processes, such as nutrient transport, cell adhesion, and signaling pathways. CD98hc is the heavy chain subunit, which covalently binds through disulfide bonds and electrostatic interactions to a number of light chains, to form six heterodimeric transporters (Rodriguez et al., 2021). For instance, CD98hc binds to the light chain of L-type amino acid transporter 1 (LAT1, SLC7A5) to form the large neutral amino acid transporter LAT1, also known as cluster of differentiation 98 (CD98), which facilitates the uptake of various amino acids such as isoleucine, leucine, methionine, valine, histidine, tyrosine, and tryptophan. Another example is the cystine/glutamate antiporter (System xC-), paired by CD98hc and SLC7A11, which facilitates the exchange of extracellular cystine for intracellular glutamate. These transport activities are crucial for supporting cellular processes like protein synthesis, energy metabolism, and antioxidant defense, and thus are vital to cell proliferation and survival. Moreover, by interacting with cell adhesion receptor integrin β1 (ITGB1), CD98hc modulates intracellular signaling (Prager et al., 2007).

CD98hc is highly expressed in tissues with high metabolic demands, such as the immune system cells and endothelial cells. The aberrant expression of CD98hc has been linked to a variety of disease mechanisms, including cancer, immune diseases, and metabolic disorders. Overexpression of CD98hc has been widely observed in many cancer types, such as breast cancer, colorectal cancer, lung cancer, pancreatic cancer, head and neck squamous cell cancer (HNSCC), and various type of hematologic malignancies (Bajaj et al., 2016; Bianconi et al., 2022; Digomann et al., 2019; Hasegawa et al., 2022; Ichinoe et al., 2021; Kaira et al., 2009; Montero et al., 2022; Poettler et al., 2013; Salter et al., 1989; Xia and Dubrovska, 2023). Its overexpression contributes to the Warburg effect, where cancer cells preferentially use glycolysis over oxidative phosphorylation, even in the presence of oxygen. The increased amino acid uptake facilitated by CD98hc supports the biosynthesis of proteins, nucleotides, and lipids, which are essential for the rapid proliferation of cancer cells (Liu et al., 2022). Moreover, the interaction between CD98hc and integrins facilitates cell adhesion and migration, processes that are crucial for metastasis (Cantor and Ginsberg, 2012). Beyond cancer, CD98hc has also been implicated in various immune-related diseases. Its involvement in the regulation of T cell activation and function suggests that modulation of its activity could provide therapeutic benefits in autoimmune diseases or in the modulation of immune responses (Nakao et al., 1993).

Given these critical roles in tumor metabolism and survival, CD98hc has gained great attention as a potential therapeutic target. Recent studies have demonstrated that inhibiting CD98 expression or function can hinder the growth of tumors in preclinical models, making it a promising candidate for targeted cancer therapy. The potential for targeting CD98hc in clinical therapies extends to the development of monoclonal antibodies, small molecules, CAR-T therapy, or RNA-based therapies that specifically inhibit its function (Nachef et al., 2021). Several studies are underway to explore small-molecule inhibitors of CD98hc as a novel class of anticancer agents (Zhang et al., 2021). Anti-CD98hc antibodies have shown promising results by significantly reducing tumor growth in preclinical models of hepatocellular carcinoma (Lim et al., 2024), and breast cancer (Montero et al., 2022). Hosen's group showed that the anti-CD98hc mAb R8H283, which targets a glycosylation site of CD98hc that was specific to myeloma but not normal hematopoietic cells (Hasegawa et. al., 2022), was effective on treatment of non-small cell lung cancer (NSCLC) by CAR-T therapy (Yaga et al., 2024). IGN523, anti-CD98hc antibody, demonstrated robust tumor growth inhibition in preclinical studies. It was evaluated in a Phase I clinical trial for acute myeloid leukemia (AML), but complete or partial responses were not observed for the treated individuals (Hayes et al., 2015; Bixby et al., 2015; NCT02040506).

While the therapeutic potential of CD98hc inhibition is promising, there are challenges in developing effective therapies. The high expression level of CD98hc on various cancer cells makes it a promising target for antibody-drug conjugates (ADCs) candidates (Montero et al., 2022). A key determinant of ADC efficacy is the tumor specificity and internalization efficiency of the target antigen. Besides high expression, its natural property of being recruited into lysosomes by the lysosomal protein LAPTM4b (Milkereit et al., 2015) can facilitate drug delivery and processing in lysosomes. However, its ubiquitous expression in normal tissues raises concerns about off-target effects and toxicity. Thus, use of CD98hc should specifically target cancers. In this regard, developing a high-affinity anti-CD98hc mAb that selectively binds to a conformational epitope present in cancer cells while sparing normal human tissues was sought.

### SUMMARY

Hybridoma MAb52-4.2 was generated by live-cell immunization (LCI) and live-cell high-throughput screening (LC-HTS) using human cancer cell lines. The MAb52-4.2 mAb produced by the hybridoma exhibited strongly binding to various types of cancer cell lines while showing weak interactions with lymphocytes in normal human PBMCs and bone marrow mononuclear cells (BMMCs). Importantly, it did not bind to normal human tissues, indicating potential tumor specificity. MAb52-4.2 mAb demonstrated high affinity (K_{D}≈2.2 nM) for the human CD98hc protein, with a notable increase in affinity when CD98hc formed a complex with its binding partners, such as SLC7A11 (also known as xCT or CCBR1).

A human-murine chimeric antibody (cAb) and a humanized antibody (HuAb), both containing a human kappa light chain constant region and a human IgG1 heavy chain constant region with free cysteine mutations (m2), were recombinantly expressed. MAb52-4.2cAbm2 and MAb52-4.2HuAbm2 retained similar binding properties to multiple cancer cell lines and recombinant CD98hc, comparable to the murine antibody counterpart. When conjugated with a cleavable linker and a payload such as monomethyl auristatin E (MMAE), MAb52-4.2cAbm2-MMAE and MAb52-4.2HuAbm2-MMAE exhibited potent antiproliferative effects across multiple cancer cell lines in vitro. Furthermore, MAb52-4.2cAbm2-MMAE effectively suppressed tumor growth in vivo across various cell line-derived xenograft (CDX) mouse models, reinforcing its potential as a promising therapeutic agent for cancer treatment.

The cDNA coding sequence of the variable region of the light chain of MAb52-4.2 mAb is shown as SEQ ID NO: 1 below.

(SEQ ID NO: 1)
CAAATTGTTCTCACCCAGTCTCCAACAATCATGTCTGCATCTCCTGGGG
AGAAGGTCACCATAGCCTGCAGTGTCAGCTCAAGTGTAAATTACATACA
CTGGGTCCAGCAGAAGCCAGGCACTTCTCCCAAACTCTGGATTTATAGC
ACGTCCAAATTGGCTTCTGGAGTCCCTGCTCGCTTCAGTGGCAGTGGCT
CTGGGACCTCTTATTCTCTCACAGTCAGCCGAATGGAGGCTGAAGATGC
TGCCACTTATTACTGCCAACAAAGGAGTAGTTACCCGTACACGTTCGGA
GGGGGGACCAAGCTGGAAATAAGA

The translated amino acid sequence of the variable region of the light chain of MAb52-4.2 mAb and MAb52-4.2cAbm2 is shown as SEQ ID NO: 2 below. Three complementarity determining regions (CDRs1 to 3, left to right, respectively shown as SEQ ID NOs: 5, 6, 7) are highlighted in bold below and underlined.

(SEQ ID NO: 2)
QIVLTQSPTIMSASPGEKVTIACSVSSSVNYIHWVQQKPGTSPKLWIYS
TSKLASGVPARFSGSGSGTSYSLTVSRMEAEDAATYYCQQRSSYPYTFG
GGTKLEIR

The cDNA coding sequence of the variable region of the heavy chain of MAb52-4.2 mAb is shown as SEQ ID NO: 3 below.

(SEQ ID NO: 3)
CAGGTCCAACTGCAGCAGCCTGGGGCTGAACTGGGCAAGCCTGGGACAT
CAGTGAAGCTGTCCTGCAAGGCTTCTGGCTACACATTCACCAGTTATTG
GATGCACTGGGTGAAGCAGAGGCCTGGACAAGGCCTTGAGTGGATTGGA
AACATTAATCCTAATGGTGGTAAGACTAACTATAATGAGAGGTTCAAGA
ATAAGGCCACACTGACTGTTGACAAATCCTCCAACACAGCCTACATGCA
GCTCACCTCCCTGACATCTGAGGACTCTGCGGTCTACTTCTGTACAAGA
GAGATAGACTGGGGCCAAGGCACCACTCTCACAGTCTCCTCA

The translated amino acid sequence of the variable region of the heavy chain of MAb52-4.2 mAb and MAb52-4.2cAbm2 is shown as SEQ ID NO: 4 below. Three complementarity determining regions (CDRs1 to 3, left to right, respectively shown as SEQ ID NOs: 8, 9, 10) are highlighted in bold and underlined below.

(SEQ ID NO: 4)
QVQLQQPGAELGKPGTSVKLSCKASGYTFTSYWMHWVKQRPGQGLEWIG
NINPNGGKTNYNERFKNKATLTVDKSSNTAYMQLTSLTSEDSAVYFCTR
EIDWGQGTTLTVSS

The designed amino acid sequence of the variable region of the light chain of MAb52-4.2HuAbm2, designated as HuVL2, is shown as SEQ ID NO: 13 below, and three antigenic determinant regions (CDRs1 to 3, left to right, respectively shown as SEQ ID NOs: 5, 6, 7) are highlighted in bold and underlined below.

(SEQ ID NO: 13)
EIVLTQSPATLSLSPGERATLACSVSSSVNYIHWVQQKPGQAPRLWIYS
TSKLASGVPARFSGSGSGTDFTLTISSLEPEDFAVYYCQQRSSYPYTFG
QGTKLEIK

The designed amino acid sequence of the variable region of the heavy chain of MAb52-4.2HuAbm2, designated as HuVH2, is shown as SEQ ID NO: 14 below, and three antigenic determinant regions (CDRs1 to 3, left to right, respectively shown as SEQ ID NOs: 8, 9, 10) are highlighted in bold and underlined.

(SEQ ID NO: 14)
QVQLVQSGAEVKKPGASVKVSCKASGYTFTSYWMHWVRQAPGQGLEWIG
NINPNGGKTNYNERFKNRATMTRDTSTSTVYMELSSLRSEDTAVYFCTR
EIDWGQGTLVTVSS

When the chimeric or humanized format of MAb52-4.2 mAb was conjugated with a cytotoxic agent to form an ADC, it efficiently inhibited tumor growth in vitro and/or in vivo. Additionally, MAb52-4.2 can be deployed as bispecific, multi-specific, or other fragments or antibody derivatives, or dual-drug ADCs, antibody radionuclide conjugates (RACs), CAR-T/NK/microphage/DC therapies, and proteolysis-targeting chimera (PROTAC), offering innovative solutions for other therapeutic applications in the treatment of fibrosis and neural diseases.

The MAb52-4.2 ADCs can also be administrated alone or in combination with other therapeutic approaches, such as immunotherapy, chemotherapy, or cell therapy, for the treatment of cancers, such as breast, lung, colorectal, gastric, pancreatic, or skin cancer, particularly those resistant to standard anti-cancer therapies.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1 to 1A-6 show that MAb52-4.2 mAb binds to the cell surfaces of all 34 tested human solid tumor cell lines and two immortalized cell lines, Beas-2B and HUVEC, as determined by FACS analyses. The meaning of the abbreviations in the figures is: MFI: median fluorescence intensity; FITC: fluorescein isothiocyanate; [mAb]: concentration of monoclonal antibody; NSCLC: non-small cell lung cancer; SCLC: small cell lung cancer; GC: gastric cancer; CRC: colorectal cancer; HCC: hepatocellular carcinoma; PDAC: pancreatic ductal adenocarcinoma; TNBC: triple-negative breast cancer; BC: breast cancer; CC: cervical cancer.

FIGS. 1B-1 and 1B-2 depict that MAb52-4.2 mAb binds to all 12 tested hematologic malignant cell lines. The meaning of the abbreviations in the figures is: AML: acute myeloid leukemia; CML: chronic myeloid leukemia; T-ALL: T cell acute lymphoblastic leukemia; B-ALL: B cell acute lymphoblastic leukemia; NHL-BL: non-Hodgkin lymphoma (Burkitt lymphoma).

FIG. 2A shows interaction between MAb52-4.2 mAb and normal human peripheral blood mononuclear cells (PBMCs), including a general population and subpopulations of lymphocytes, monocytes, neutrophils, and platelets.

FIG. 2B shows interaction between MAb52-4.2 mAb and normal human bone marrow mononuclear cells (BMMCs) and the gated subpopulations based on size and internal complexity. BMMCs were categorized using a forward scatter (FSC) versus side scatter (SSC) plot to distinguish large cells, medium cells with high complexity (high-medium), medium cells with low complexity (low-medium), and small cells.

FIG. 2C shows interaction between MAb52-4.2 mAb and normal human cord blood mononuclear cells (CB-MNCs), including a general population and subpopulations of lymphocytes, monocytes, neutrophils, and platelets.

FIG. 2D illustrates no interaction between MAb52-4.2 mAb and normal cynomolgus monkey PBMCs, including a general population and subpopulations of lymphocytes, monocytes, neutrophils, and platelets.

FIG. 3A shows the IHC result of formalin-fixed, paraffin-embedded (FFPE) NSCLC section reacted with MAb52-4.2 mAb. Tissues were visualized under a microscope at 10× magnification. Blue staining corresponds to hematoxylin counterstaining, which visualizes cells and nuclei, while positive staining appears in brown.

FIG. 3B displays the IHC results of normal human tissue microarray (TMA) stained with MAb52-4.2 mAb (upper panel) and a table summarizing the staining results along with the corresponding tissue locations on the TMA slide (lower panel). The TMA slide contains 90 different sections, with every three consecutive spots being the same tissue from three distinct donors. Placenta serves as a positive control.

Blue staining corresponds to hematoxylin counterstaining, which visualizes cells and nuclei, while positive staining appears in brown.

FIG. 4A shows the scores of four best hits identified by two independent immunoprecipitation (IP) and liquid chromatography with tandem mass spectrometry (LC-MS/MS) analysis. Accession numbers correspond to UniProtKB/Swiss-Prot IDs, and deduced peptide sequences are highlighted in bold. PEP: posterior error probability; PSMs: peptide spectrum matches; #AAs: number of amino acids.

FIG. 4B shows the amino acid sequence of CD98hc, mapped with the peptide sequences derived from the LC-MS/MS results highlighted in bold.

FIGS. 5A to 5C illustrate that MAb52-4.2 mAb has a weak interaction with the rhCD98hc-ECD-His monomer, but a strong interaction with the rhSLC3A2/SLC7A11 virus-like protein (VLP) complex in ELISA assays, suggesting MAb52-4.2 mAb binds to a conformational epitope. Binding of mAb to the antigens was probed with 1:4,000 diluted Peroxidase AffiniPure™ Goat Anti-Mouse IgG, Fcγ fragment specific (Jackson ImmunoResearch Laboratories). A450 represents absorbance at 450-nm wavelength.

FIG. 5A shows the low binding signal between MAb52-4.2 mAb and rhCD98hc-ECD-His. Anti-6×His-tag mAb serves as a positive control to confirm proper coating of the antigen. Each well was immobilized with 15 ng of rhCD98hc-ECD-His.

FIG. 5B shows that MAb52-4.2 mAb interacts with a mixture of cell lysates from various cancer cell lines in a dose-dependent manner. Anti-6×His-tag mAb had non-specific binding at a high concentration, whereas the isotype control mAb did not bind to the cell lysates. Each well was immobilized with cell lysates containing approximately 2.0 μg proteins.

FIG. 5C shows the dose-dependent binding of MAb52-4.2 mAb to rhSLC3A2/SLC7A11-VLP but not to VLP protein isotype control.

FIG. 6 shows no detectable band in cancer cell lysates by MAb52-4.2 mAb in Western blot. MAb52-4.2 mAb detected a weak band with 1.0 μg rhCD98hc-ECD-His but no band with 1.0 μg of rhSLC3A2/SLC7A11-VLP under non-reducing condition. Anti-human β-actin mAb-HRP served as a loading control. Numbers indicate the molecular weights in kilodalton (kDa) of Broad Multi Color Pre-Stained Protein Standard (GenScript). Arrows indicate the locations of rhCD98hc-ECD-His and β-actin bands, respectively.

FIG. 7 presents the aligned sensorgram traces depicting the association and dissociation curves for MAb52-4.2 mAb binding to rhCD98hc-ECD-His, extracted from raw data analyzed by ForteBio Octet® QK^{e} System. Anti-Mouse Fc Capture (AMC) biosensors were immobilized with 100 nM MAb52-4.2 mAb. The rhCD98hc-ECD-His protein was loaded at concentrations of 200, 100, 50, 25, and 12.5 nM by 2-fold serial dilution to allow association and dissociation for 15 min and 30 min, respectively.

FIG. 8 illustrates a dose-dependent binding of MAb52-4.2 mAb to the surface of HEK293FT cells expressing the full-length human CD98hc protein assayed by FACS. Surface binding was detected using 1:800 diluted R-Phycoerythrin (R-PE) AffiniPure™ Goat Anti-Mouse IgG, Fcγ Fragment Specific (Jackson ImmunoResearch Laboratories). No signal was detected with the parental HEK293FT cells, which does not express CD98hc (lower panel).

FIG. 9 shows the relative expression levels of the SLC3A2 gene encoding CD98hc in representative cancer cell lines, as determined by qRT-PCR analysis. Data were normalized to RPLP0 mRNA and expressed as fold change relative to healthy human PBMC controls. Values represent the mean±SD of triplicate samples. *P<0.05, **P<0.01 (Student's t test).

FIG. 10 illustrates the effect of MAb52-4.2 mAb on the invasion of various cancer cell lines. “ctrl” represents the baseline of isotype control mAb-treated groups. Positive values indicate increased invasion, while negative values indicate decreased invasion. Data are represented as the mean±SD of triplicate samples.

FIGS. 11A to 11H are fluorescent images showing internalization of FITC-labeled MAb52-4.2 mAb in eight different human cancer cell lines. Cells were incubated with 1 μg/mL MAb52-4.2 mAb-FITC, or an isotype control mAb-FITC for one hour at 4° C. After PBS wash, the cells were incubated at 37° C. with 5% CO₂ for 15 min, 30 min, or 2 hours. The dynamics of antibody binding and internalization of each antibody are shown vertically at four different conditions, with 4° C. as the baseline of antibody binding. The pictures were taken at 40× magnification with the same exposure time. Scale bars indicate 100 μm.

FIG. 12 illustrates the dose-dependent binding of MAb52-4.2cAbm2 to the surface of various human cancer cell lines, as determined by FACS analysis. Human PBMCs served as a negative control. cAbm2: chimeric antibody with double cysteine mutations in each heavy chain constant region (Fc); [Ab]: antibody concentration.

FIG. 13 is the aligned sensorgram traces showing association and dissociation curves for MAb52-4.2cAbm2 binding to rhCD98hc-ECD-His, extracted from raw data by the ForteBio software. The Octet® QK^{e} System was equipped with Anti-Human Fc Capture (AHC) biosensors to immobilize 100 nM MAb52-4.2cAbm2. The rhCD98hc-ECD-His was loaded at concentrations of 200, 100, 50, 25, and 12.5 nM by 2-fold serial dilution to allow association and dissociation for 15 min and 30 min, respectively.

FIG. 14 shows the SDS-PAGE analysis of MAb52-4.2cAbm2 with (+) or without (−) MMAE conjugation under both reducing and non-reducing conditions. Numbers to the left indicate the molecular weights in kDa of GenScript Broad Multi Color Pre-Stained Protein Standard (middle lane). Each lane was loaded with approximately 2 μg protein.

FIGS. 15-1 to 15-7 illustrate the inhibitory effects of MAb52-4.2cAbm2-MMAE on the proliferation of various human cell lines, as determined by cell counting assays. The non-cell binding isotype control, Isotype ctrl cAbm2-MMAE, served as the proliferation baseline for each cell line. A₄₅₀: absorbance at 450 nm. Data represent the mean±SD of triplicate samples.

FIGS. 16A to 16F illustrate the antitumor effects of MAb52-4.2cAbm2-MMAE on six cancer cell line-derived xenograft (CDX) mouse models. MAb52-4.2cAbm2-MMAE, isotype control cAbm2-MMAE, or PBS was injected intraperitoneally (i.p.) with the dose as indicated on Day 0. Data represents average tumor volume±SD of five mice per group (n=5). PBS: Dulbecco's phosphate buffer saline; MMAE: monomethyl auristatin E.

FIG. 17A depicts the comparison of the parental murine mAb light chain variable region (MoVL) with the select human germline acceptor IGKV3-11*01 and two humanized MAb52-4.2 light chain VLs (HuVL1 and HuVL2). CDRs of MAb52-4.2 mAb are highlighted in bold and underline. Grey and black bars on top of the sequences are the Kabat and Chothia definitions of CDRs, respectively. Back mutations in frameworks are italicized and highlighted.

FIG. 17B depicts the comparison of the parental murine mAb heavy chain variable region (MoVH) with the select human germline acceptor IGHV1-46*01 and two humanized MAb52-4.2 heavy chains VHs (HuVH1 and HuVH2). CDRs of MAb52-4.2 mAb are highlighted in bold and underline. Grey and black bars on top of the sequences are the Kabat and Chothia definitions of CDRs, respectively. Back mutations in frameworks are italicized and highlighted.

FIG. 18A shows the curves of dose-dependent binding to eight representative cancer cell lines by MAb52-4.2HuAbm2 are comparable with that of MAb52-4.2cAbm2, as determined by FACS analysis.

FIG. 18B depicts binding of MAb52-4.2HuAbm2 to HEK293FT cells expressing CD98hc are comparable with that of MAb52-4.2cAbm2, as determined by FACS analysis.

FIG. 19 shows a competition ELISA assay, where binding between rhCD98hc-ECD-His and HRP-conjugated MAb52-4.2 mAb (MAb52-4.2mAb-HRP) was inhibited by unlabeled MAb52-4.2cAbm2 and MAb52-4.2HuAbm2, to a similar extent as their parental murine MAb52-4.2 mAb. The isotype control cAbm2, which recognizes a non-cell surface antigen, did not compete with MAb52-4.2mAb-HRP. The plate was immobilized with 20 ng/well rhCD98hc-ECD-His.

FIG. 20 shows the aligned sensorgram traces depicting the association and dissociation curves and the data table of the binding kinetics between MAb52-4.2HuAbm2 and rhCD98hc-ECD-His at 2-fold serial dilutions (200, 100, 50, 25, and 12.5 nM). The Octet® QK^{e} System was equipped with Anti-Human Fc Capture (AHC) biosensors to immobilize 100 nM MAb52-4.2HuAbm2.

FIGS. 21A to 21F are fluorescent images showing internalization of CF488-conjugated MAb52-4.2HuAbm2 by six different human cancer cell lines. Cells were incubated with 1 μg/mL MAb52-4.2HuAbm2—CF488, or an isotype control cAbm2—CF488 for one hour at 4° C. to obtain the baselines of antibody binding for each cell line. Antibody internalization was observed at 15 min, 30 min, and 2 hours after incubation at 37° C. The pictures were taken at 40× magnification with the same exposure time. Scale bars indicate 100 μm.

FIG. 22 shows the SDS-PAGE analysis of MAb52-4.2HuAbm2 with (+) or without (−) MMAE conjugation under both reducing and non-reducing conditions. Numbers to the left indicate the molecular weights in kDa of GenScript Broad Multi Color Pre-Stained Protein Standard (left lane). Each lane was loaded with approximately 2 μg protein.

FIG. 23 compares the surface binding of MAb52-4.2cAbm2 and MAb52-4.2HuAbm2, with or without MMAE conjugation, to six human cancer cell lines, as determined by FACS analyses. Each cell line represents a distinct cancer type. An isotype control chimeric antibody (Isotype ctrl cAbm2), with or without MMAE conjugation, served as the negative control.

FIG. 24 shows in vitro cell counting assays comparing the efficacies of MMAE-conjugated MAb52-4.2cAbm2 and MAb52-4.2HuAbm2 in inhibiting the proliferation of four representative human cancer cell lines. Isotype control cAbm2-MMAE served as the proliferation baseline for each cell line. Data represent the mean±SD of triplicate samples.

### DETAILED DESCRIPTION

### Definitions

The section headings used herein are for organizational purposes only and are not to be construed as limiting the subject matter described. All patents and applications listed herein are incorporated by reference.

Unless defined otherwise, all technical and scientific terms used herein have the same meaning as is commonly understood by one of ordinary skill in the art. All patents, applications, published applications, and other publications are incorporated by reference in their entirety. In the event that there is a plurality of definitions for a term herein, those in this section prevail unless stated otherwise.

The term “ADCs” refers to antibody-drug conjugate, wherein a binding agent, including a monoclonal antibody, fragment or derivative is conjugated with a drug. Suitable drugs in an ADC include any substance having biological or detectable activity, for example, therapeutic agents, detectable labels, binding agents, and prodrugs, which are metabolized to an active agent in vivo. Suitable drugs in the conjugates preferably include anti-tumor agents, including Monomethyl auristatin E (MMAE) or another anti-tumor agent, for example, a cytotoxin, including maytansine or a derivative thereof, or a derivative of Auristatin, epothilone or a derivative thereof, paclitaxel or a derivative thereof, or a vinca alkaloid compound; or further including: Combretastatin A-4 phosphate, Combretastatin A-4 and its derivatives, indole-sulfa compounds, vinca alkaloids compounds such as vinblastine, vincristine, vindesine, vinorelbine, vinflunine, vinglycinate, anhydrovinblastine, dolastatins 10 and analogues, halichondrin B and Eribulin, indole-3-oxalyl amides, substituted indol-3-oxalyl amides, podophyllotoxins, 7-diethylamino-3-(2′-benzoxazolyl)-coumarin (DBC), discodermolide, Laulimalide; DNA topoisomerase inhibitors such as camptothecin and its derivatives, mitoxantron; mitoguazone; nitrogen mustard analogues such as Chlorambucil, Chlomaphazine, cyclophosphamide, Estramustine, ifosfamide, Mustine, Nitromin, Melphalan, Novembichin, Phenamet, Phenesterine, Prednimustine, Trofosfamide, Uramustine; nitrosoureas such as Carmustine, streptozotocin, Fotemustine, Lomustine, Nimustine, Ranimustine; antibiotics such as the enediyne antibiotics, Dynemicin, Esperamicin, Neocarzinostatin, Aclacinomycin, Actinomycin, Anthroamycin, Azaserine, Bleomycins, actinomycin C, Carabicin, Idarubicin, Carzinophilin, Carminomycin, Actinomycin D, Daunorubicin, Doxorubicin, 6-diazo-5-oxo-L-norleucine, Adriamycin, Epirubicin, Esorubicin, Idarubicin, Marcellomycin, Mitomycins, Mycophenolic acid, Nogalamycin, Olivomycin, Peplomycin, Bofeimeisu, Puromycin, Adriamycin-Fe, Rodorubicin, Streptonigrin, Streptozocin, Tubercidin, Ubenimex, Zinostatin, Zorubicin; folic acid analogues such as Denopterin, Methotrexate, Pteropterin, Trimetrexate, Edatrexate; Purine analogues such as Fludarabine, 6-mercaptopurine, Thiamiprine, Thioguanine; pyrimidine analogues such as Ancitabine, Gemcitabine, Enoxaparin, Azacitidine, 6-Azauridine, Carmofur, Cytarabine, dideoxyuridine, deoxy-fluorouridine, Fluoruridine; androgens such as Calusterone, Dromostanolone propionate, Epitiostanol, Mepitiostance, Testolactone; anti-adrenal compounds such as Aminoglutethimide, Mitotane, Trilostane; trichothecenes such as T-2 toxin, verracurin A, Roridin A and Anguidine; arizidines such as Benzodopa, Carboquone, Meturedopa and Uredopa; platinum analogs such as Cisplatin, Carboplatin, Oxaliplatin, Miriplatin, Etoposide; anti-androgens such as Flutamide, Nilutamide, Bicalutamide, Leuprolide and Goserelin; protein kinase and proteasome inhibitors.

The binding agent is preferably bound to the anti-tumor agent using a linker, or other binding methods can be used. In some embodiments, linkers may be used in the conjugate bonding which can be composed of flexible residues like glycine and serine (one example being four glycine residues followed by a serine, repeated; or simply repeated glycine residues) so that the adjacent protein domains are free to move relative to one another. If one desires to maintain distance between domains, so they cannot interact, rigid linkers are preferred; one example being (repeating): glutamic acid, three alanine residues, lysine. Another rigid linker is (repeating): any amino acid residue and proline. Linkers can be either non-cleavable (e.g., thioether, SMCC, PEG linkers), or cleavable linkers, such as valine-citrulline (Val-Cit, VC) dipeptide, glutamic acid-valine-citrulline (Glu-Val-Cit, GVC) tripeptide and disulfide linkers. U.S. Pat. No. 9,310,373 “Molecular conjugate” discloses a number of hydrazide thiol linkers, and making conjugates with them. U.S. Pat. No. 8,518,891 discloses a linker with an aminoarylmethyl or aminoheteroaryl moiety. Bi-functional linkers have also been described. See U.S. Pat. No. 11,040,084.

The antibody in the ADC can be a whole antibody or a fragment thereof. The binding agents of the invention include MAb52-4.2-like or MAb52-4.2-derived antibodies, antibody fragments, fusion proteins, or an antibody-derived or modified chimeric antibody receptor (CAR), including single-chain variable fragments (scFv) fused to intracellular signaling domains, e.g., the zeta chain of CD3 (CD3ζ). CAR is preferably expressed in CAR immune effector cells including but not limited to T cells, natural killer (NK) cells, microphages, and dendritic cells (DCs).

The term binding agent(s) includes an antibody (both are interchangeably used in singular or plural form) which is an immunoglobulin molecule capable of binding to a target antigen, such as an antigen on lung cancer cells, through at least one antigen recognition site, located in the variable region of the immunoglobulin molecule. As used herein, the term “antibody” encompasses not only intact (i.e., full-length) monoclonal antibodies like MAb52-4.2, but also antigen-binding fragments thereof (such as Fab, Fab′, F(ab′)₂, Fv, Fd, rIgG, single chain (scFv) or sc(Fv)₂, mutants thereof, fusion proteins comprising an antibody portion, humanized antibodies (like MAb52-4.2HuAbm2), chimeric antibodies (like MAb52-4.2cAbm2), diabodies, multi-specific antibodies (e.g., bispecific antibodies), single domain antigen binding (SDAB) molecules, a VH or VL domain, or a VHH domain, and any other modified configuration of the immunoglobulin molecule that comprises an antigen recognition site of the required specificity, including glycosylation variants of antibodies, amino acid sequence variants of antibodies, and covalently modified antibodies. Also included are antibody-drug conjugates.

An antibody includes antibodies of any class, such as IgD, IgE, IgG, IgA, or IgM (or a sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2.

Antibodies described herein can be murine, rat, human, or any other origin (including chimeric or humanized antibodies, including as described in U.S. Pat. No. 7,317,091B2, and including such antibodies generated by affinity maturation). In some examples, the antibody comprises a modified constant region, such as a constant region that is immunologically inert, e.g., does not trigger complement mediated lysis, or does not stimulate antibody-dependent cell mediated cytotoxicity (ADCC).

Humanized antibodies refer to forms of non-human (e.g. murine) antibodies that are specific chimeric immunoglobulins, immunoglobulin chains, or antigen-binding fragments thereof that contain sequences derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody will also optimally comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody. Humanized antibodies may also involve affinity maturation. See Jones et al., Nature, 321:522-525 (1986); Riechmann et al., Nature, 332:323-327 (1988); Verhoeyen et al., Science, 239:1534-1536 (1988).

A chimeric antibody includes a heavy chain constant region and a light chain constant region from a human antibody. See, e.g., Morrison et al. (1984) Proc. Natl. Acad. Sci. USA 81, 6851; Neuberger et al. (1984) Nature 312, 604; and Takeda et al. (1984) Nature 314:452. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. See U.S. Pat. No. 4,816,567.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

### Formulations

Antibodies and Binding Agents and ADCs can be prepared in a formulation for administration to a subject. A lyophilized formulation is preferred, which as a first step, requires preparing a pre-lyophilized formulation. The amount of ADC in the pre-lyophilized formulation is determined taking into account the desired dose volumes, mode(s) of administration, etc. The protein is generally present in solution. For example, the protein may be present in a pH-buffered solution at a pH from about 4-8, and preferably from about 5-7. Exemplary buffers include histidine, phosphate, Tris, citrate, succinate and other organic acids. The buffer concentration can be from about 1 mM to about 20 mM, or from about 3 mM to about 15 mM, depending, for example, on the buffer and the desired isotonicity of the formulation (e.g. of the reconstituted formulation). The preferred buffer is histidine as it can have lyoprotective properties. Succinate is also a useful buffer.

The lyoprotectant is added to the pre-lyophilized formulation. In preferred embodiments, the lyoprotectant is a non-reducing sugar such as sucrose or trehalose. The amount of lyoprotectant in the pre-lyophilized formulation is generally such that, upon reconstitution, the resulting formulation will be isotonic, as preferred, though hypertonic reconstituted formulations may also be suitable. In addition, the amount of lyoprotectant must not be too low such that an unacceptable amount of degradation/aggregation of the protein occurs upon lyophilization.

Where the lyoprotectant is a sugar (such as sucrose or trehalose) and the protein is an antibody, exemplary lyoprotectant concentrations in the pre-lyophilized formulation are from about 10 mM to about 400 mM, and preferably from about 30 mM to about 300 mM, and most preferably from about 50 mM to about 100 mM.

The ratio of protein to lyoprotectant is selected for each protein and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole antibody, and preferably from about 200 to about 1000 moles of lyoprotectant to 1 mole antibody, including from about 200 to about 600 moles of lyoprotectant to 1 mole antibody.

In preferred embodiments, it has been found to be desirable to add a surfactant to the pre-lyophilized formulation. Alternatively, or in addition, the surfactant may be added to the lyophilized formulation and/or the reconstituted formulation. Exemplary surfactants include nonionic surfactants such as polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g. Pluronics, PF68 etc). The amount of surfactant added is such that it reduces aggregation of the reconstituted protein and minimizes the formation of particulates after reconstitution. For example, the surfactant may be present in the pre-lyophilized formulation in an amount from about 0.001-0.5%, and preferably from about 0.005-0.05%.

A mixture of the lyoprotectant (such as sucrose or trehalose) and a bulking agent (e.g. mannitol or glycine) may be used in the preparation of the pre-lyophilization formulation. The bulking agent may allow for the production of a uniform lyophilized cake without excessive pockets therein.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; metal complexes (e.g. Zn-protein complexes); biodegradable polymers such as polyesters; and/or salt-forming counterions such as sodium. In addition, various adjuvants such as are commonly used in the art may be included. Considerations for the inclusion of various components in pharmaceutical compositions are described, e.g., in Gilman et al. (Eds.) (1990), Goodman and Gilman's: The Pharmacological Basis of Therapeutics, 8th Ed., Pergamon Press, which is incorporated herein by reference in its entirety.

The pharmaceutical compositions and formulations described herein are preferably stable, so as to retain its physical and chemical stability and integrity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes.

After the protein, lyoprotectant and other optional components are mixed together, the formulation is lyophilized. Many different freeze-dryers are available for this purpose such as Hull50® (Hull, USA) or GT20® (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation.

Typically, the shelf temperature for the primary drying will range from about-30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hours). A secondary drying stage may be carried out at about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial. As a general proposition, lyophilization will result in a lyophilized formulation in which the moisture content thereof is less than about 5%, and preferably less than about 3%.

At the desired stage, typically when it is time to administer the protein to the patient, the lyophilized formulation may be reconstituted with a diluent such that the protein concentration in the reconstituted formulation is preferably similar to that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

Alternatively, a non-lyophilized formulation may be used, including ADCs and any of the well-known carriers, excipients, buffers, stabilizers, preservatives, adjuvants and other additives described herein and well known in the art.

### Dosages and Administration

The ADC formulations described above can be administered to a subject (e.g., a human) in need of the treatment via a suitable route, such as administration by intravenous, intramuscular, intraperitoneal, intracerebrospinal, subcutaneous, intracutaneous, intraarticular, intrasynovial, intrathecal, intradermal, intratumoral, intranodal, intramedulla, oral, inhalation or topical routes; or it may be administered orally, by inhalation spray, topically, rectally, nasally, buccally, vaginally or via an implanted reservoir; and in any case, as a bolus or by continuous infusion over a period of time; or via injectable depot routes of administration such as using 1-, 3-, or 6-month depot injectable or biodegradable materials and methods.

Commercially available nebulizers for liquid formulations, including jet nebulizers and ultrasonic nebulizers are useful for administration. Liquid formulations can be directly nebulized and lyophilized powder can be nebulized after reconstitution. Alternatively, ADCs can be aerosolized using a fluorocarbon formulation and a metered dose inhaler, or inhaled as a lyophilized and milled powder. Where CAR is deployed in the invention, compositions of immune effector cells (e.g., T cells, NK cells) may be injected directly into a tumor, lymph node, or site of infection, or elsewhere.

The subject to be treated by the methods described herein can be a mammal, more preferably a human. Mammals include, but are not limited to, farm animals, sport animals, pets, primates, horses, dogs, cats, mice and rats.

An “effective amount” refers to the amount of ADC required to confer therapeutic effect on the subject, either alone or in combination with one or more other active agents. Effective amounts vary, depending on the particular condition being treated, the severity of the condition, the individual patient parameters including age, physical condition, size, gender and weight, the duration of the treatment, the nature of concurrent therapy (if any), the specific route of administration and like factors, all of which are well known to those of ordinary skill in the art and can be addressed with no more than routine experimentation. It is generally preferred that a maximum dose of the individual components or combinations thereof be used, that is, the highest safe dose according to sound medical judgment. A lower dose or tolerable dose for medical reasons, psychological reasons or other reasons, is also appropriate.

Empirical considerations, such as the ADC half-life, generally will contribute to the determination of the dosage. For example, ADCs that are compatible with the human immune system, e.g., those including humanized antibodies or fully human antibodies, may be used to prolong half-life of the antibody and to prevent the ADC being attacked by the host's immune system. Frequency of administration may be determined and adjusted over the course of therapy, and is generally, but not necessarily, based on treatment and/or suppression and/or amelioration and/or delay of cancer. Alternatively, sustained continuous release formulations of antibody may be appropriate. Various formulations and devices for achieving sustained release are known in the art.

In one example, dosages for an ADC as described herein may be determined empirically in individuals who have been given one or more administration(s) of the antibody. Individuals are given incremental dosages of the antibody. To assess efficacy of the ADC, an indicator of the disease (e.g., tumor growth) can be followed according to routine practice.

Generally, for administration of any of the ADCs described herein, an initial candidate dosage can be extrapolated from the experiments described below. For repeated administrations over several days or longer, depending on the condition, the treatment is sustained until a desired suppression of symptoms occurs or until sufficient therapeutic levels are achieved to alleviate the cancer. An exemplary dosing regimen comprises administering an initial higher dose, followed by a lower maintenance dose. However, other dosage regimens may be useful, depending on the pattern of pharmacokinetic decay that the practitioner wishes to achieve. For example, dosing from one-four times a week is contemplated. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

Conventional methods, known to those of ordinary skill in the art of medicine, can be used to administer the pharmaceutical composition to the subject, depending upon the treatment goal and the cancer site.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like).

For intravenous injection, water soluble ADCs can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients.

Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an ADC is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., WO 00/53211 and U.S. Pat. No. 5,981,568,

In another embodiment of the present disclosure, an article of manufacture is provided which contains any of the pharmaceutical compositions and formulations described herein (e.g., comprising an ADC) and provides instructions for its use and/or reconstitution. The article of manufacture comprises a container. Suitable containers include, for example, bottles, vials (e.g., dual chamber vials), syringes (such as dual chamber syringes) and test tubes. The container may be formed from a variety of materials such as glass or plastic. The container holds the formulation and the label on, or associated with, the container may indicate directions for reconstitution and/or use. For example, the label may indicate that the formulation is reconstituted to particular protein concentrations. The container holding the formulation may be a multi-use vial, which allows for repeat administrations (e.g., from 2-6 administrations) of the reconstituted formulation. The article of manufacture may further comprise a second container comprising a suitable diluent (e.g., BWFI). Upon mixing of the diluent and the lyophilized formulation, the final protein concentration in the reconstituted formulation will generally be at least 50 mg/mL. The article of manufacture may further include other materials desirable from a commercial and user standpoint, including other buffers, diluents, filters, needles, syringes, and package inserts with instructions for use.

### EXAMPLES

The following Examples are not limiting but only exemplary. Unless otherwise specified, all cell lines were purchased from American Type Culture Collection (ATCC, Manassas, VA) and cultured in Dulbecco s Modified Eagle's Medium (DMEM; Thermo Fisher Scientific, Waltham, MA) supplemented with 10% (v/v) Gibco fetal bovine serum (FBS; Thermo Fisher Scientific) at 37° C. in a humidified atmosphere with 5% CO₂. Standard methods were used to manipulate DNA and RNA described in Sambrook et al., Molecular cloning: A laboratory manual; Cold Spring Harbor Laboratory Press, Cold Spring Harbor, New York, 1989. All commercial reagents and kits were used according to the manufacturer's instructions unless otherwise stated.

### Example 1

Generation of MAb52-4.2 mAb Bound Highly with Human Solid Tumor Cell Lines

### A. Generation of Hybridoma Clones by Live-Cell Immunization and Live-Cell Screening

To obtain mAbs specifically targeting human malignant tumors, a mixture of three triple-negative breast cancer (TNBC) cell lines, BT-20, BT549, and Hs 578T, was used for live-cell immunization (LCI) and live-cell high-throughput screening (LC-HTS) as described previously (see: U.S. Pat. No. 11,939,391B2, US20210139602A1, WO2014146487A1, WO2017114204). Hybridoma culture supernatants were screened for binding to the surfaces of these three cell lines and counter-screened with human peripheral blood mononuclear cells (PBMCs) from healthy adult donors (Stanford Blood Center, Palo Alto, CA) using a fluorescence-activated cell sorting (FACS) assay. Briefly, cancer cell lines cultured in DMEM/10% (v/v) FBS were detached using 0.2% (w/v) Ethylenediaminetetraacetic acid (EDTA) and mixed with PBS. The cells were collected by centrifugation at 500×g and blocked with ice-cold PBS containing 1.0% (w/v) bovine serum albumin (BSA; Thermo Fisher Scientific). Hybridoma culture supernatants were incubated with the cells at 4° C. for 30 minutes (min). Surface antibody binding was detected using 1:800 diluted Fluorescein AffiniPure™ Goat Anti-Mouse IgG, Fcγ Fragment Specific (Jackson ImmunoResearch Laboratories, West Grove, PA). After three washes with ice-cold PBS/1.0% (w/v) BSA, cells were fixed with 4.0% (w/v) paraformaldehyde. FACS was performed using BD FACSCalibur™ Flow Cytometer equipped with High Throughput Sampler (HTS), and data were analyzed using FlowJo™ 10.8 software (Becton Dickinson, San Jose, CA).

Hybridoma colonies that exhibited high fluorescence signals in binding to TNBC cell lines, but very weak or no binding signal to PBMCs in the FACS assay, were selected for expansion, weaning from conditional medium, and subcloning following standard protocols (Kohler & Milstein, Nature 1975, 256:495-497; Winter & Milstein, Nature 1991, 349:293-299). MAbs secreted by these hybridoma subclones were purified from the culture supernatants by affinity chromatography using MabSelect™ SuRe™ LX Protein A resin (Cytiva, Wilmington, DE) and buffer-exchanged into phosphate-buffered saline (PBS, pH7.2; Cytiva). Antibody concentrations were determined by absorbance at 280 nM (A280) using a Nanodrop™ 2000 Spectrophotometer (Thermo Fisher Scientific). The purity and integrity of the mAbs were assessed by sodium dodecyl-sulfate polyacrylamide gel electrophoresis (SDS-PAGE) on 4-12% SurePAGE™ Bis-Tris gels (GenScript, Piscataway, NJ) stained with SimplyBlue™ SafeStain (Thermo Fisher Scientific). The isotype of each mAb was determined using IsoStrip™ Mouse Monoclonal Antibody Isotyping Kit (Roche Molecular Systems, Pleasanton, CA).

B. FACS Analysis of mAbs in Binding to Cancer Cell Surfaces

The reactivity of purified mAbs was confirmed by assessing their binding to representative cancer cell lines from different cancer types with FACS assays. Purified mAbs with serial dilutions were incubated with individual cell lines at 4° C. for 30 min, followed by detection with Fluorescein AffiniPure™ Goat Anti-Mouse IgG, Fcγ Fragment Specific. To evaluate specificity and potential off-target effects, multiple batches of fresh PBMCs from distinct healthy donors were stained in parallel as negative controls. Additionally, frozen human bone marrow mononuclear cells (BMMCs) from healthy donors and human cord blood mononuclear cells (CB-MNCs) purchased from HumanCells Bioscience (Milpitas, CA) were carefully thawed and stained to assess the safety profile of the mAbs as therapeutic candidates.

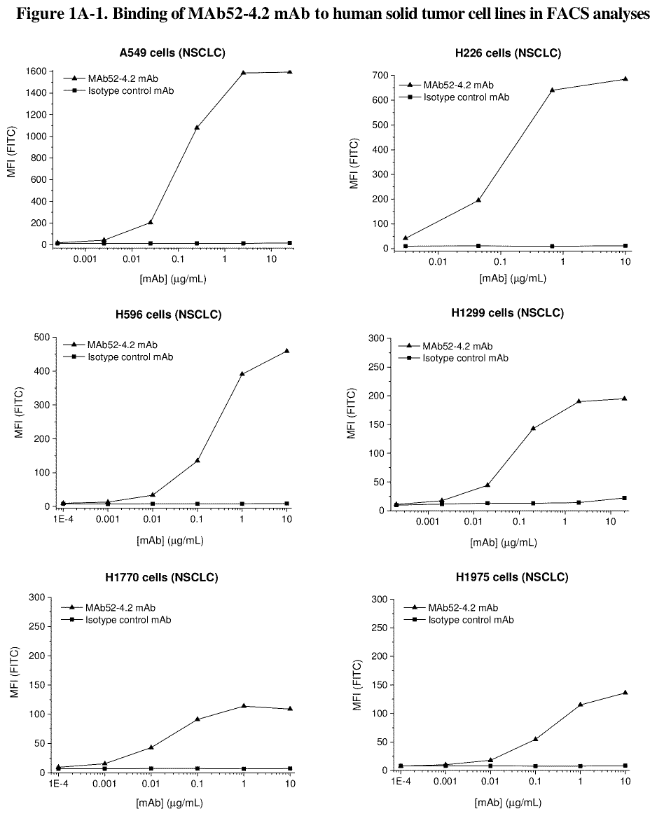

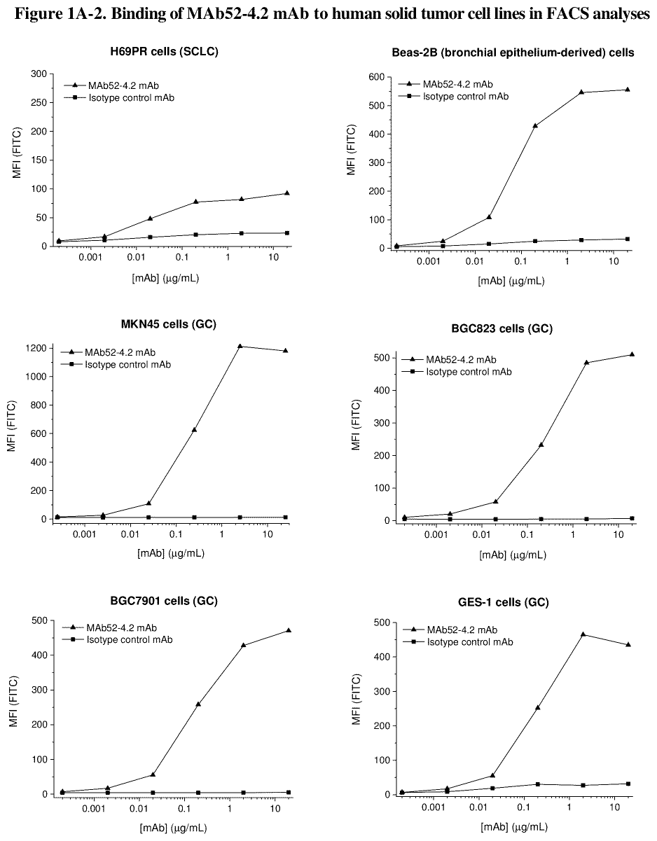

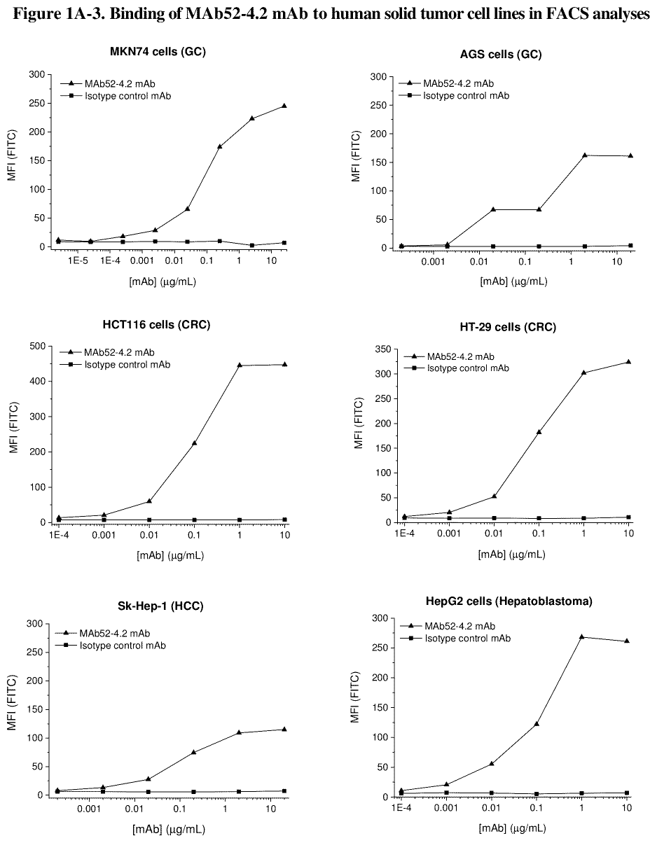

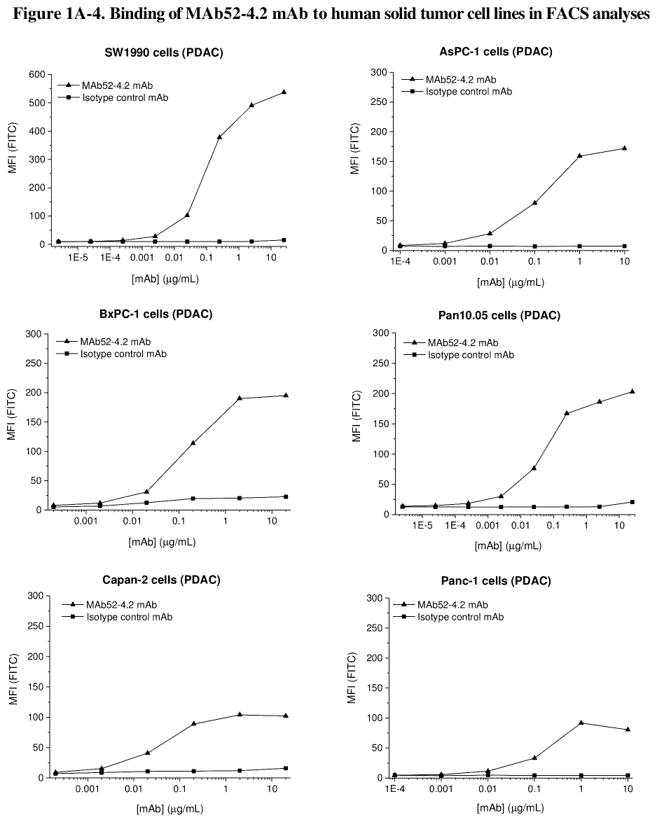

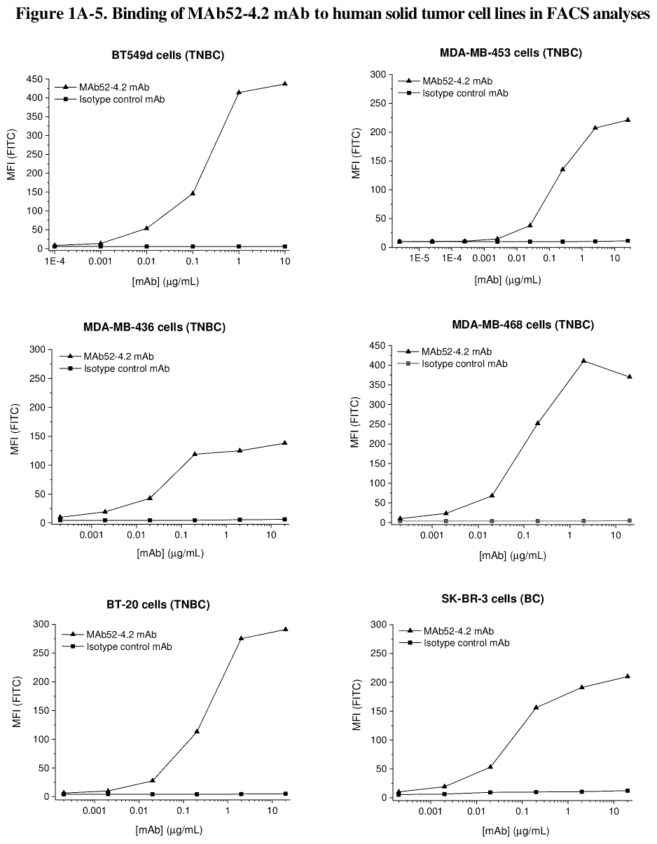

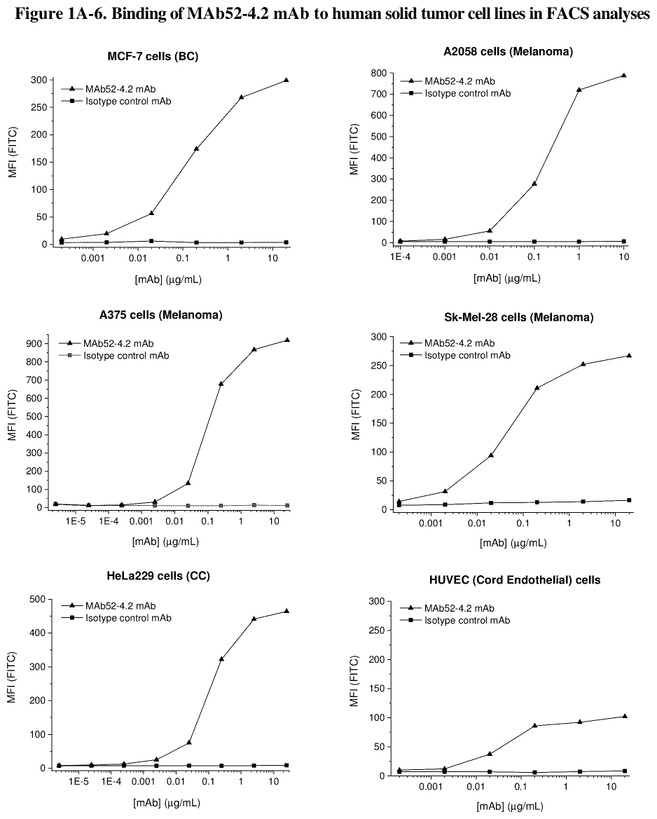

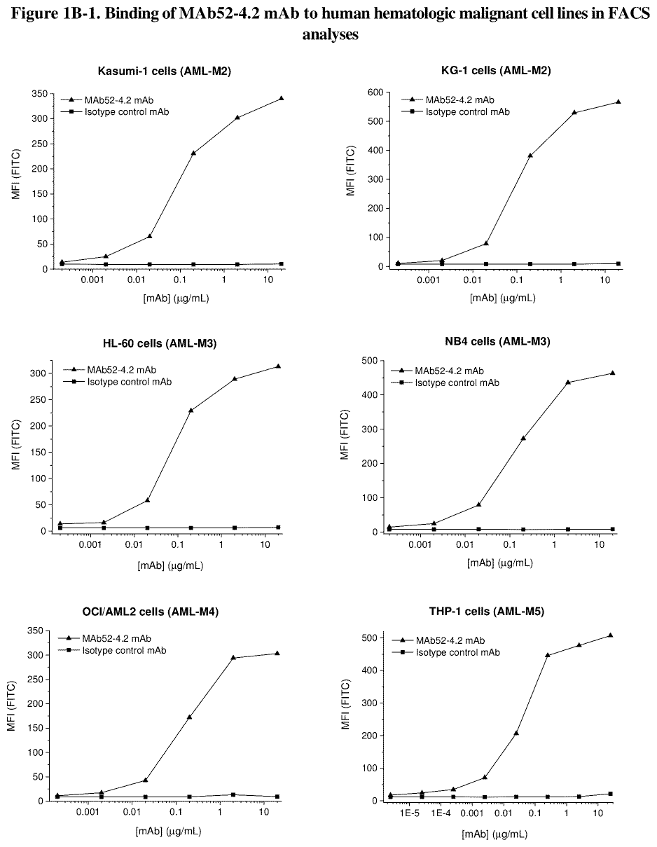

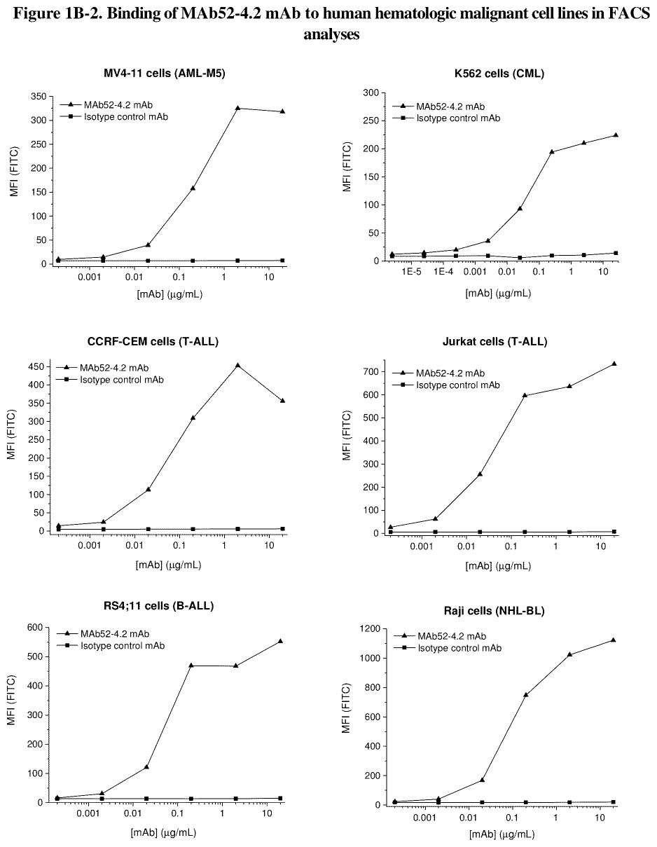

Among all purified mAbs, MAb52-4.2 mAb exhibited a high binding capacity to nearly all types of cancer cell lines, including lung cancer (NSCLC and SCLC), gastric cancer (GC), colorectal cancer (CRC), hepatocellular carcinoma (HCC), pancreatic ductal adenocarcinoma (PDAC), breast cancer (BC), melanoma, cervical cancer (CC), leukemia (AML, ALL, and CML), and lymphoma (FIG. 1A and FIG. 1B), suggesting that its target is widely expressed across both solid and liquid tumors and may play a role in cancer cell proliferation and metastasis.

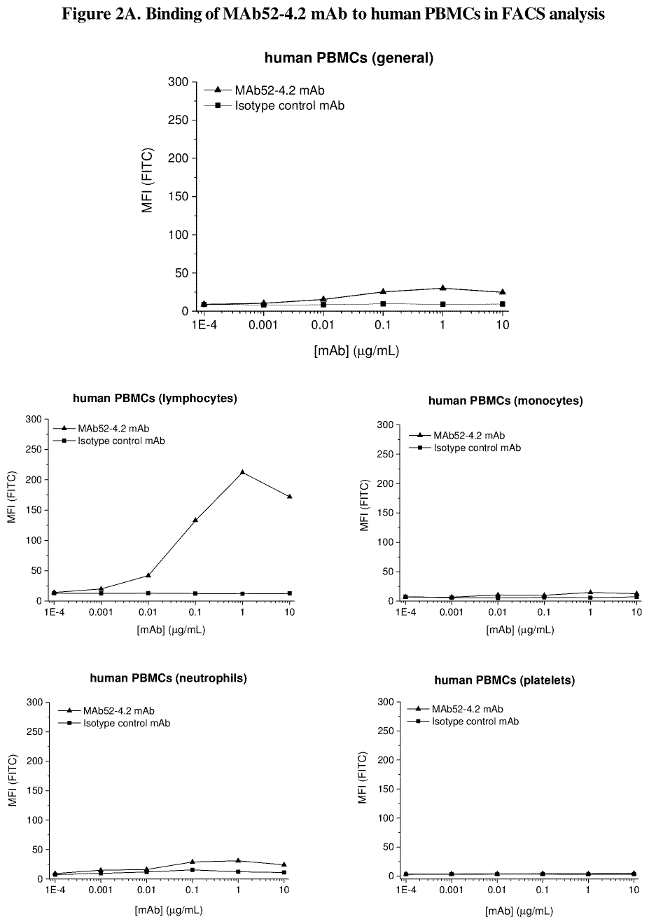

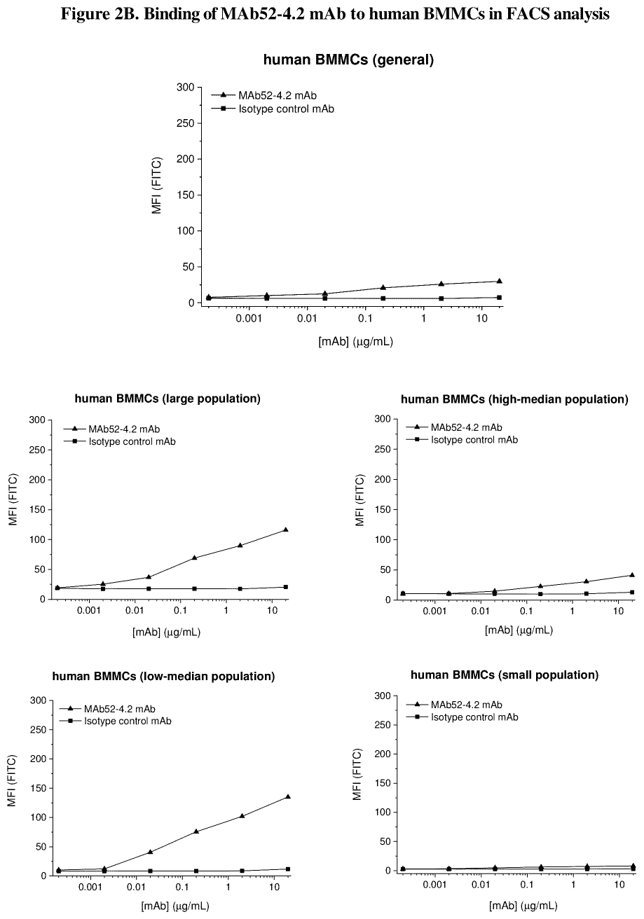

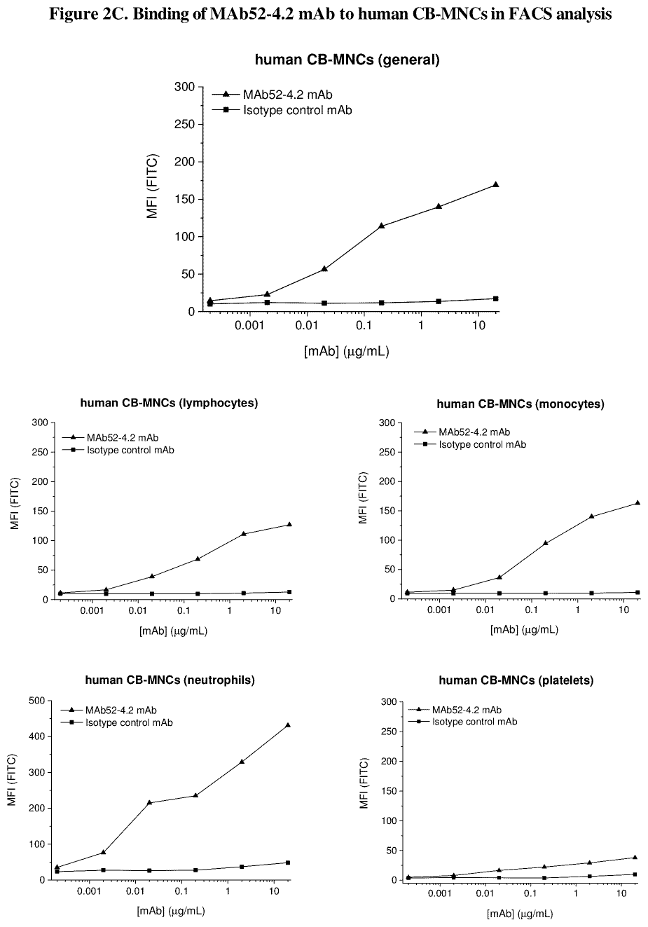

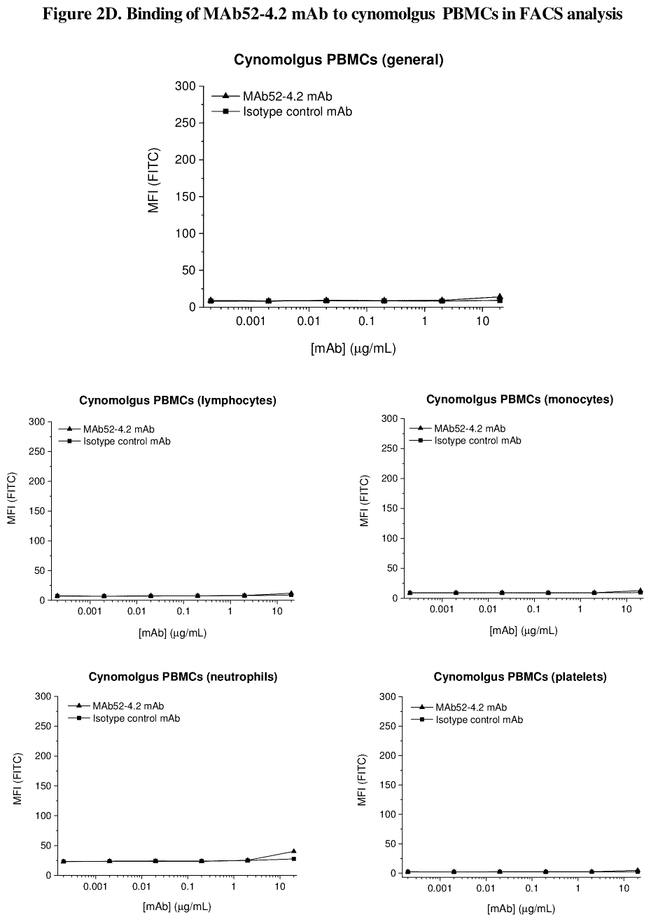

In FACS analyses with normal human cells, MAb52-4.2 mAb bound to lymphocytes in human PBMCs (FIG. 2A) and BMMCs (FIG. 2B). Additionally, it boud to CBMCs (FIG. 2C), which contain a significant portion of stem cells, indicating that its target is present in early-stage cells. This suggests that MAb52-4.2 mAb's target could be a shared biomarker for both progenitor cells and cancer cells. Notably, MAb52-4.2 mAb did not cross-react with PBMCs isolated from cynomolgus monkeys (FIG. 2D), which may have implications for its species specificity.

Isotype typing revealed that MAb52-4.2 mAb consists of a murine kappa light chain and an IgG1 heavy chain, and therefore, a murine IgG1-Kappa isotype control mAb was included in all binding assays for MAb52-4.2 mAb.

### C. IHC Assay of MAb52-4.2 mAb in Binding to Normal Human Tissues

To assess whether MAb52-4.2 mAb interacts with normal human tissues beyond lymphocytes and CB-MNCs, an immunohistochemistry (IHC) assay was performed using FDA standard formalin-fixed paraffin-embedded (FFPE) tissue microarray (TMA) slides (Biochain, Newark, CA). Each TMA slide contains 30 different human tissue samples, each donated from three distinct donors. Among them, 29 are normal tissues, and the placenta sections serve as a positive control. Lung cancer (NSCLC) FFPE sections were used to validate the IHC protocol and the optimal concentration of MAb52-4.2 mAb for the assay. Tissue sections were deparaffinized, rehydrated through graded alcohols, and subjected to heat-induced antigen retrieval (HIER), followed by blocking with 10% (v/v) horse serum (Vector Laboratories, Newark, CA). Sections were incubated overnight at 4° C. with 100 μg/mL MAb52-4.2 mAb. Antibody binding was detected using VECTASTAIN® Elite® ABC-HRP Kit, Peroxidase (Mouse IgG) from Vector Laboratories. Samples were counterstained with Mayer's hematoxylin solution (MilliporeSigma, St. Louis, MO), mounted in VectaMount® AQ Mounting Medium (Vector Laboratories), and coverslipped. Tissue sections were imaged and scanned using the BZ-X800 All-in-One Fluorescence Microscope (Keyence, Itasca, IL).

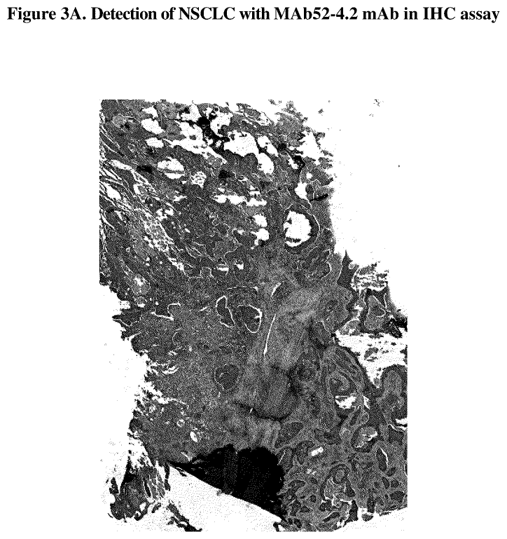

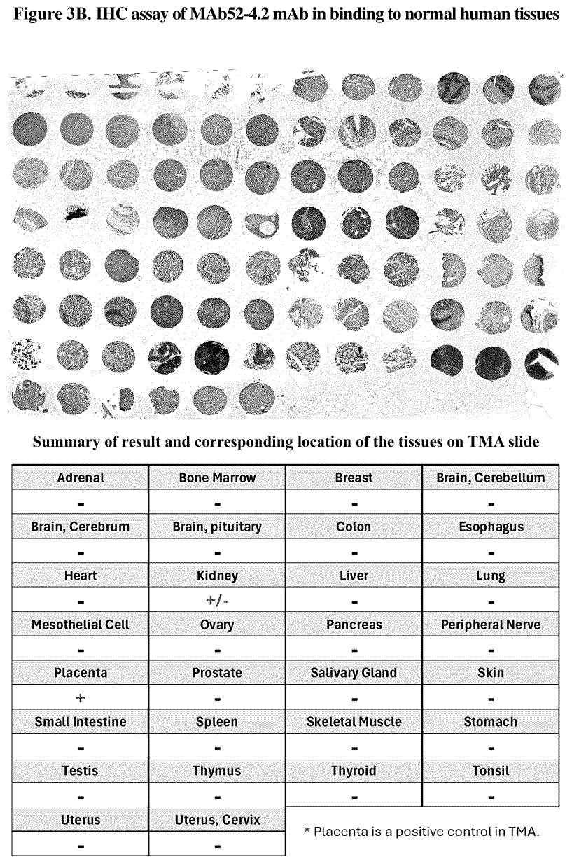

IHC analysis revealed that lung cancer tissue sections stained with 100 μg/mL (or 0.67 μM) of MAb52-4.2 mAb exhibited a strong brown precipitate, indicating high expression levels of the target antigen (FIG. 3A). In contrast, MAb52-4.2 mAb showed no staining or only weak background signals in 28 of 29 normal tissue types examined on the normal TMA slide (FIG. 3B). The only exception was the kidney, where weak signals were observed. However, this was likely due to the reaction between endogenous biotin in the kidney and the horse radish peroxidase (HRP) conjugated to the secondary antibody, a common artifact observed in IHC with therapeutic antibodies.

These findings confirm a clear distinction in target protein expression between cancerous and normal tissues, reinforcing the potential of MAb52-4.2 mAb as a therapeutic drug candidate for cancer treatment.

### Example 2

### Identification and Verification of Human CD98hc as the Target of MAb52-4.2 mAb

### A. Target Identification by Immunoprecipitation (IP) and Mass Spectrometry (MS)

To identify the target of MAb52-4.2 mAb, it was conjugated to Dynabeads™ Protein-A (Thermo Fisher Scientific) for immunoprecipitation (IP), or to HRP with Lightning-Link® HRP Conjugation Kit (Abcam, Waltham, MA), according to the manufacturers' protocols. Cancer cell lines that showed strong reactivity with MAb52-4.2 mAb were lysed with radioimmunoprecipitation assay (RIPA) buffer (Thermo Fisher Scientific), and the lysates of mixed cancer cell lines were incubated with the Dynabeads™-MAb52-4.2 mAb complex at room temperature (RT) for 30 min. The complex was washed sequentially with 0.5% (v/v) TritonX-100/PBS and PBS. The immunoprecipitated (IP) protein samples were separated by SDS-PAGE under a non-reducing condition and transferred to a nitrocellulose membrane (MilliporeSigma). The membrane was blocked with PBS containing 0.05% (v/v) Tween®-20 (PBST) and 1% (w/v) BSA and probed with MAb52-4.2 mAb-HRP, followed by detection using SuperSignal™ West Pico PLUS Chemiluminescent Substrate (Thermo Fisher Scientific).

Nevertheless, MAb52-4.2 mAb-HRP failed to detect any specific bands in the Western blot analysis, suggesting that the target(s) is likely a conformational epitope that was denatured during the Western blot process. This indicates that the antibody recognizes a three-dimensional structure rather than a linear peptide sequence. To further investigate, the MAb52-4.2 mAb IP samples were digested directly with trypsin and Lys-C protease and analyzed by liquid chromatography with tandem mass spectrometry (LC-MS/MS, Innomics, San Jose, CA). The resulting data were searched against the latest reviewed human database from Uniprot (https://www.uniprot.org/) using Mascot.

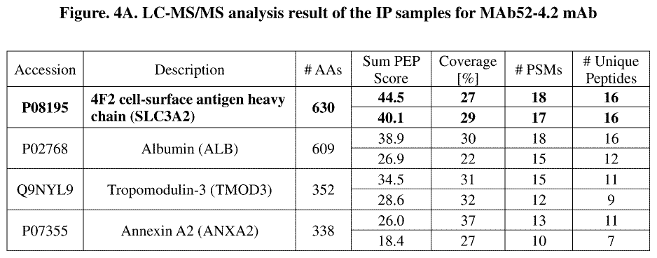

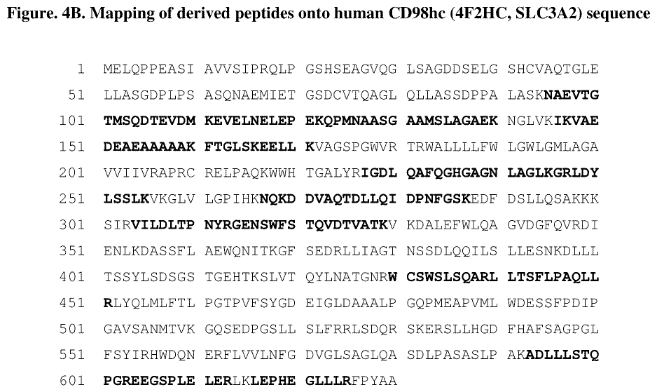

FIG. 4A lists the most confidently identified proteins detected from two IP samples. CD98hc, also known as 4F2HC or SLC3A2 (UniProtKB/Swiss-Prot: P08195), had the highest scores with 27% to 29% sequence coverages (FIG. 4B). Other proteins identified were excluded based on their properties and subcellular locations.

### B. Verification of Interaction Between MAb52-4.2 mAb and CD98hc by ELISA Assay

A direct ELISA assay using a recombinant human CD98hc extracellular domain (ECD, Arg 206-Ala 630) with an N-terminal His tag, namely rhCD98hc-ECD-His, expressed in HEK293 cells (Sino Biological US, Wayne, PA) was performed to assess its reactivity with MAb52-4.2 mAb. The antigen was immobilized on Immulon® microtiter plates (Thermo Fisher Scientific) at 15 ng/well, washed with PBST and blocked with PBST/1% (w/v) BSA at RT for one hour. MAb52-4.2 mAb, anti-6×His-tag mAb (Proteintech, Rosemont, IL), or isotype control mAb was added in serial dilutions and incubated for one hour. After washing, the plates were probed with 1:4,000 diluted Peroxidase AffiniPure™ Goat Anti-Mouse IgG, Fcγ fragment specific (Jackson ImmunoResearch Laboratories) for 30-60 min. Following thorough wash, 3,3′,5,5′-Tetramethylbenzidine (TMB) was added to allow color development for 15 min and then stopped with 0.05 M sulfuric acid. Absorbance at 450 nm (A450) was measured using the SpectraMax Microplate Reader (Molecular Device, San Jose, CA).

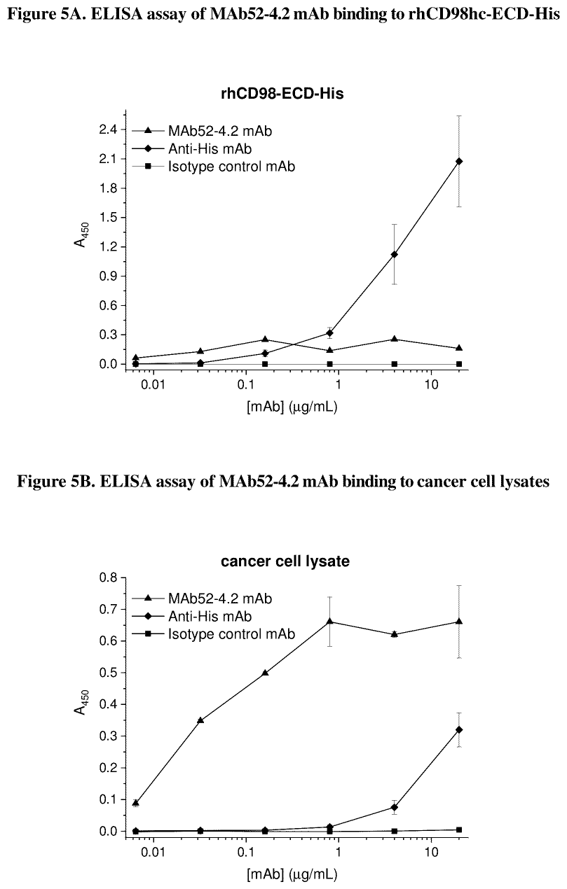

The binding of MAb52-4.2 mAb to rhCD98hc-ECD-His was very weak (FIG. 5A), raising concerns about its interaction with the purified antigen. Successful antigen coating was confirmed using anti-6×His mAb, though its binding affinity was low (EC₅₀≥5 μg/mL), and mouse anti-human CD98 mAb (Proteintech) verified the identity of the antigen. Despite multiple assay repetitions and the inclusion of rhCD98hc-ECD-His from a different manufacturer (AcroBiosystems, Newark, DE), results remained consistent, which challenged the IP and LC-MS/MS results.

However, MAb52-4.2 mAb strongly bound to immobilized proteins from cancer cell lysates, with an EC₅₀ of approximately 0.02 μg/mL (0.13 nM) (FIG. 5B), indicating a high affinity for its native target.

The discrepancy between binding to purified rhCD98hc-ECD-His versus cell lysates in ELISA assays and the failure to detect specific bands from the IP samples in Western blot analysis suggested that MAb52-4.2 mAb might recognize a conformational epitope in CD98hc that was disrupted during Western blot conditions. Given that CD98hc is a component of multiple membrane transporters, it is likely that the tertiary or quaternary structure required for MAb52-4.2 mAb binding is not properly presented in the monomeric rhCD98hc-ECD-His protein.

To examine this hypothesis, a full-length human SLC3A2 (CD98hc) and SLC7A11 heterodimer protein, expressed from HEK293 cells in the form of virus-like particles (VLPs) to preserve the native conformations of these membrane proteins, was purchased from Acro Biosystems, along with the VLP protein isotype control to rule out non-specific interactions. ELISA results demonstrated strong, dose-dependent binding of MAb52-4.2 mAb to rhSLC3A2/SLC7A11-VLP (FIG. 5C), confirming the LC-MS/MS identification of CD98hc as a target. The EC₅₀ for MAb52-4.2 mAb and rhSLC3A2/SLC7A11-VLP interaction was below 0.01 μg/mL (0.067 nM), implicating its high binding affinity.

### C. Western Blot Confirming a Conformational Binding Epitope on CD98hc

For further verification, rhSLC3A2/SLC7A11-VLP and rhCD98hc-ECD-His were electrophoresed along with cancer cell lysates under non-reducing condition, and reacted with MAb52-4.2 mAb for one hour in a Western blot analysis. After washing, Peroxidase AffiniPure™ Goat Anti-Mouse IgG, Fcγ fragment specific, and HRP-conjugated anti-β-actin mAb (Proteintech) were used for probing, followed by reacting with SuperSignal™ West Pico PLUS Chemiluminescent Substrate (Thermo Fisher Scientific).

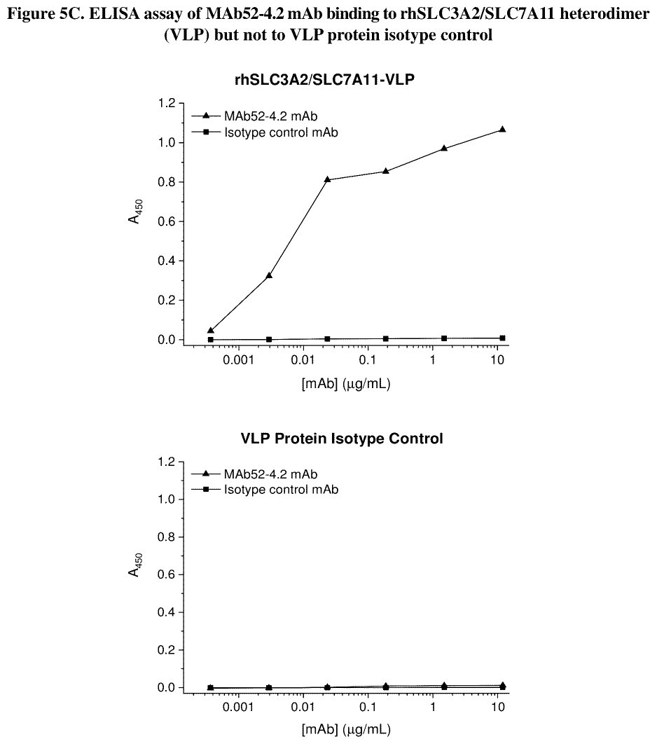

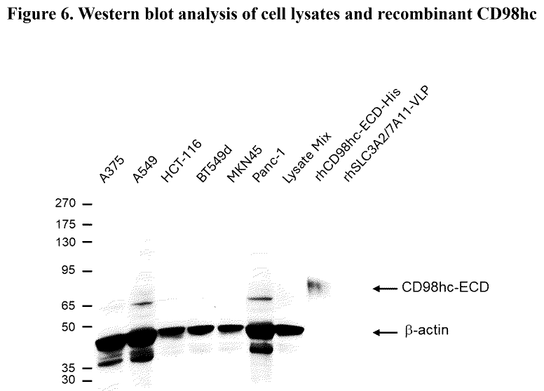

As expected, MAb52-4.2 mAb failed to detect specific bands in cancer cell lysates, likely due to denaturation of the conformational epitope during electrophoresis (FIG. 6). Weak detection of rhCD98hc-ECD-His despite a 1.0 μg loading further supported that MAb52-4.2 mAb binds a conformation-dependent epitope. The rhSLC3A2/SLC7A11-VLP complex was not detected, possibly due to its large molecular size preventing migration through the gel. Notably, rhCD98hc-ECD-His migrated at 80 kDa instead of 47.7 kDa under non-reducing conditions due to glycosylation.

These findings strongly suggest that MAb52-4.2 mAb binds a conformational epitope on the rhSLC3A2&SLC7A11 heterodimer, which was disrupted by SDS-PAGE sample preparation. The marked difference in binding to rhSLC3A2&SLC7A11-VLP versus rhCD98hc-ECD-His supports this hypothesis. However, due to the lack of commercially available CD98hc complex proteins, we are unable to determine whether MAb52-4.2 mAb specifically targets the SLC3A2&SLC7A11 heterodimer, or other CD98hc-containing heterodimers at the current stage.

D. Binding Kinetics and Affinity Between MAb52-4.2 mAb and rhCD98hc-ECD-his Determined by Octet

The binding kinetics and affinity of MAb52-4.2 mAb to rhCD98hc-ECD-His was determined using Bio-Layer Interferometry (BLI) label-free technology with an Octet® QK^{e} System (Molecular Devices, San Jose, CA). Anti-Mouse IgG Fc Capture (AMC) Biosensors (Sartorius, Fremont, CA) were immobilized with 100 nM MAb52-4.2 mAb for 10 min, followed by a 5-min wash with 1× Kinetic Buffer (Sartorius). After the baseline stabilized, sensors loaded with MAb52-4.2 mAb were exposed to rhCD98hc-ECD-His for 15 min to obtain the association curve, followed by 30 min of dissociation in 1× Kinetic Buffer. The assay was repeated multiple times using 2-fold serial dilutions of rhCD98hc-ECD-His, ranging from 200 nM to 12.5 nM, in 1× Kinetic Buffer. Kinetic data were processed with average referencing and fit to a 1:1 interaction model using Octet® Data Analysis software (version 10) to determine the association rate constant (kₒₙ) and dissociation rate constant (k_{off} or k_{dis}). The equilibrium dissociation constant (K_{D}) in molar units (M) was calculated using the formula K_{D}=k_{off}/kₒₙ. The biosensors were regenerated with 10 mM glycine, pH 1.70.

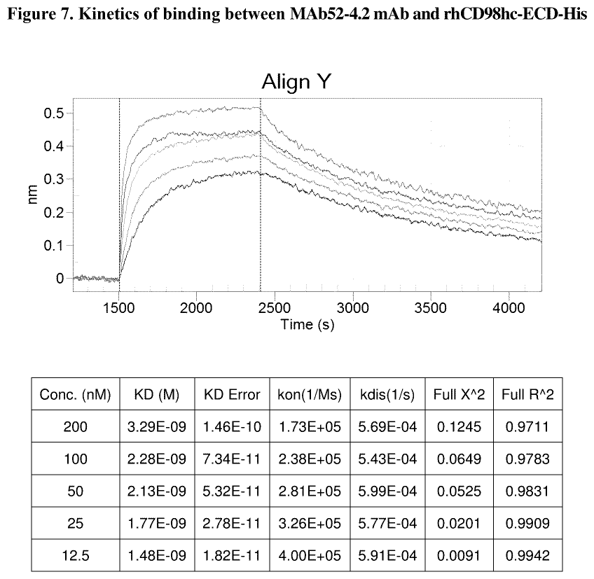

The sensorgram illustrating the association and dissociation between MAb52-4.2 mAb and rhCD98hc-ECD-His is shown in FIG. 7. A fast association rate and a moderate dissociation rate suggest a relatively high affinity. The affinity (K_{D}) calculated was approximately 2.2×10⁻⁹ M.

### E. Binding of MAb52-4.2 mAb to HEK293 Cells Expressing CD98hc on Cell Surface

To confirm the direct interaction between MAb52-4.2 mAb and CD98hc, a stable HEK293 cell line expressing recombinant human CD98hc was established. HEK293FT cells (Thermo Fisher Scientific) were transfected with the pCMV3-CD98-GFPSpark plasmid (Sino Biological), which encodes the full-length human CD98hc cDNA with a C-terminal green fluorescence protein (GFP) tag. Transfected cells were seeded into 96-well tissue culture plates containing complete culture medium supplemented with 200 μg/mL hygromycin (MilliporeSigma) for selection. Single clones exhibiting green fluorescence were expanded, and expression of the CD98hc-GFP fusion protein was screened via FACS using mouse anti-human CD98 mAb (Proteintech), followed by R-Phycoerythrin (R-PE) AffiniPure™ Goat Anti-Mouse IgG, Fcγ fragment specific (Jackson ImmunoResearch) as the secondary antibody. The HEK293FT-CD98hc cell lines with varying expression levels were cryopreserved for further analysis.

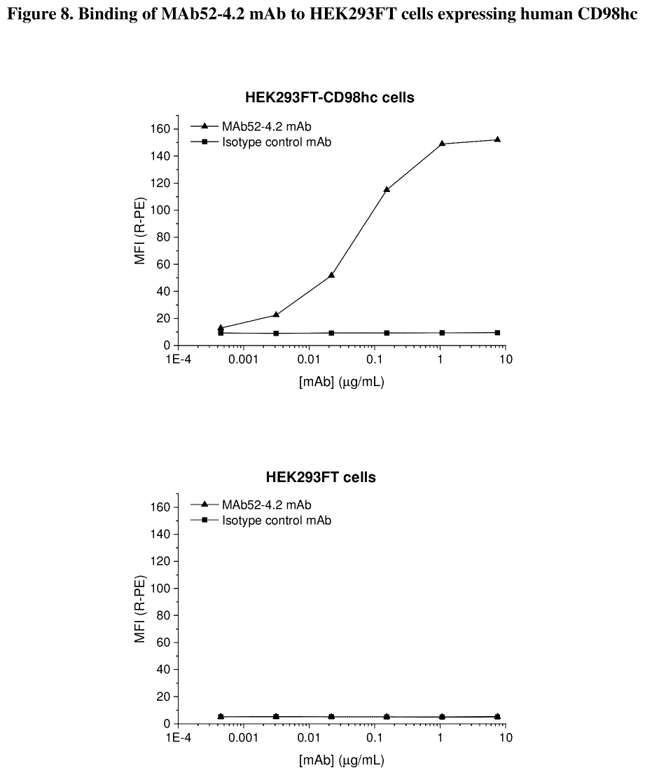

A dose-dependent binding curve of MAb52-4.2 mAb to the surface of HEK293FT-CD98hc cells, but not to the parental HEK293FT cells, was observed (FIG. 8). The HEK293 expression system preserves the native folding and post-translational modifications (PTMs) of full-length CD98hc, as well as its assembly of quaternary structure with other transporter subunits, and therefore, allows proper exposure of the conformational epitope to be accessible by MAb52-4.2 mAb. This result reinforced CD98hc to be the specific target for MAb52-4.2 mAb.

F. Detection of SLC3A2 Gene Expression in Cancer Cell Lines by qRT-PCR

CD98hc is known to widely expressed in normal tissues at both mRNA and protein levels. However, MAb52-4.2 mAb exhibited no or very weak binding to normal cells or tissues, but strong binding to various cancer cell lines and cancerous tissues, suggesting that its targeting epitope on CD98hc is highly related to cancer. It was unclear whether the different expression levels between normal and cancer cells were regulated at the gene or protein levels. As a result, the relative expression levels of the SLC3A2 gene encoding CD98hc were measured in various cancer cell lines using quantitative reverse transcription-polymerase chain reaction (qRT-PCR) to evaluate the correlation between gene expression and the abundance of the targeting epitope.

Six cell lines representing different types of cancer were used for qRT-PCR analysis. MAb52-4.2 mAb exhibited stronger binding to A375 (melanoma), A549 (NSCLC), BT549d (TNBC), HCT-116 (CRC), and MKN45 (GC; AffiGen, Baileys Harbor, WI) cells, and weaker binding to Panc-1 (PDAC) cells than lymphocytes (FIGS. 1A and 2A). The cells were cultured in DMEM supplemented with 10% (v/v) FBS until reaching exponential phase, detached, and washed with PBS. Total RNA was isolated from the cells using Quick-RNA™ Miniprep Kit (Zymo Research, Irvine, CA), and residual genomic DNA (gDNA) was removed by DNase I digestion (Thermo Fisher Scientific) according to the manufacturer's protocol. First-strand cDNA was synthesized at 25° C. for 10 min, 37° C. for 120 min, and 85° C. for 5 min using the High Capacity cDNA Reverse Transcription Kits (Thermo Fisher Scientific) in a 20-μL reaction containing 1.0 μg of total RNA, 2.0 μL of 10×RT buffer, 4 mM dNTP mix, 2.0 μL of RT Random Primers, 1.0 μL of MultiScribe™ Reverse Transcriptase, and 1.0 μL of RNase Inhibitor. Real-time PCR was performed on the ABI7800 Real Time PCR System (Thermo Fisher Scientific) using Luna Universal qPCR Master Mix (New England Biolabs, Ipswich, MA) in a 20-μL reaction with 0.2 μL of first-strand cDNA as the template. The cycling conditions were as follows: 95° C. for 60 sec, followed by 40 cycles of 95° C. for 15 sec and 60° C. for 30 sec. Relative mRNA expression levels were calculated using the comparative cycle threshold (2^{-ΔΔCT}) method (Livak and Schmittgen, Methods 2001). Ribosomal protein lateral stalk subunit P0 (RPLP0) served as the housekeeping control. The primer sequences for qPCR were:

SLC3A2 Forward:
(SEQ ID NO: 15)
5′-CCAGAAGGATGATGTCGCTCAG-3′
SLC3A2 Reverse:
(SEQ ID NO: 16)
5′-GAGTAAGGTCCAGAATGACACGG-3′
RPLP0 Forward:
(SEQ ID NO: 17)
5′-GGCGACCTGGAAGTCCAACT-3′
RPLP0 Reverse:
(SEQ ID NO: 18)
5′-CCATCAGCACCACAGCCTTC-3′

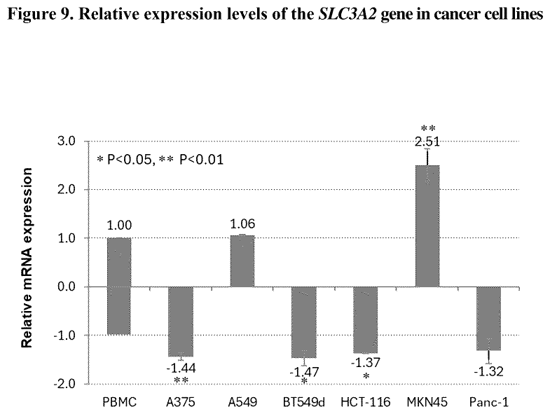

Compared with normal human PBMCs, the SLC3A2 gene was significantly upregulated in MKN45 cell line, with 2.5-fold increase, whereas downregulated in A375, BT549d, and HCT-116 cells (FIG. 9). No significant difference was found with A549 cells, though they showed the highest expression of the epitope targeted by MAb52-4.2 mAb. Thus, the differences in MAb52-4.2 mAb binding between cell lines do not relate to the SLC3A2 gene expression.

### Example 3

### In Vitro Effects of MAb52-4.2 mAb on Cancer Cell Lines

### A. Cell Invasion Assay of MAb52-4.2 mAb

MAb52-4.2 mAb did not show significant effect on cancer cell proliferation (data not shown). However, a cell invasion assay using the QCM ECMatrix Cell Invasion Assay Kit (MilliporeSigma) suggested that MAb52-4.2 mAb interferes with cancer cell invasion. This was done with A375, A549, BT549d, HCT-116, MKN45, and Panc-1 cell lines, which were seeded at a density of 3×10⁵ cells/well in serum-free DMEM supplemented with 1.0% (w/v) BSA into the 24-well plate provided by the kit. The cells were treated with 150 μg/mL of MAb52-4.2 mAb or an isotype control mAb and incubated overnight at 37° C. with 5% CO₂ and humidity. Invasive cells that migrated through the ECM layer were stained according to the manufacturer's instructions and quantified by measuring absorbance at 560 nm (A560) using a microplate reader. Each sample was measured in triplicates.

For each cell line, the difference in number of cells that invaded through the ECM matrix between the MAb52-4.2 mAb-treated group (test) and isotype control mAb-treated group (control) was calculated the following formula:

Percent change in invasive cells (%)=[(A₅₆₀ test−A₅₆₀ control)/A₅₆₀ control]×100.

Isotype control samples were used as the baseline, represented as 0±standard deviation (SD) of triplicated samples. A positive result indicates an increase in cell invasion, whereas a negative result signifies a percentage of reduction in cell invasion.

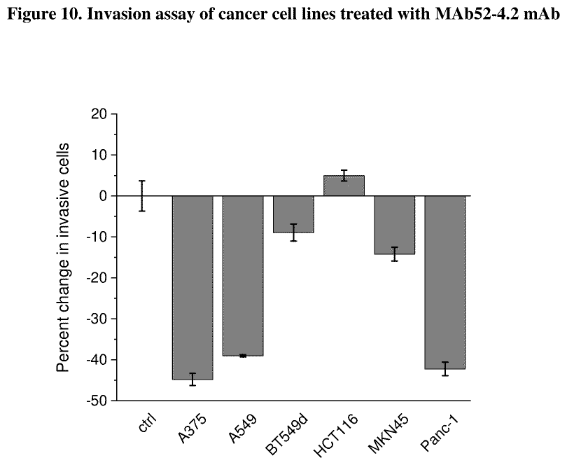

After 24 hours of co-incubation with MAb52-4.2 mAb, A375, A549, MKN45, and Panc-1 cells exhibited a significant reduction in the number of cells that migrated across the ECMatrix™ membrane (FIG. 10). Although MAb52-4.2 mAb did not demonstrate cytotoxic effect on cancer cells, it may partially inhibit cancer metastasis.

### B. Internalization Assay of MAb52-4.2 mAb by Cancer Cell Lines

To assess whether binding of MAb52-4.2 mAb to the conformational epitope of CD98hc could trigger the internalization of CD98hc, MAb52-4.2 mAb and an isotype control mAb were conjugated with fluorescein (FITC) using Abcam's FITC Conjugation Kit (Abcam). This was done by incubating the mAbs with Modifier Reagent and FITC Mix at RT for 3 hours, followed by quenching of any unbound FITC. Cell lines were cultured in DMEM medium supplemented with 10% (v/v) FBS and plated at optimal densities ranging from 1 to 5×10⁴ cells per well in four Falcon flat-bottomed 96-well tissue culture microplates (Corning, Corning, NY). The cells were incubated overnight at 37° C. with 5% CO₂ and humidity. The cells were pre-cooled to 4° C. and then co-incubated with 0.1 μg/mL MAb52-4.2mAb-FITC or Isotype control mAb-FITC at 4° C. to allow surface binding. One hour later, the cells were rinsed twice with ice-cold PBS to remove the unbound mAb-FITC. One plate was subsequently stained for 15 min at 4° C. with 4′,6-Diamidino-2-Phenylindole, dihydrochloride (DAPI; Biotium, Fremont, CA), rinsed with ice-cold PBS, and then photographed using the BZ-X800 All-in-one Fluorescence Microscope (Keyence, Itasca, IL). Meanwhile, warm culture medium was added to the remaining three plates and incubated at 37° C. for 15 min, 30 min, 2 hours, respectively, to promote internalization of the mAb-antigen complexes. These plates were also stained with DAPI for 15 min at 37° C. prior to PBS wash and imaging.

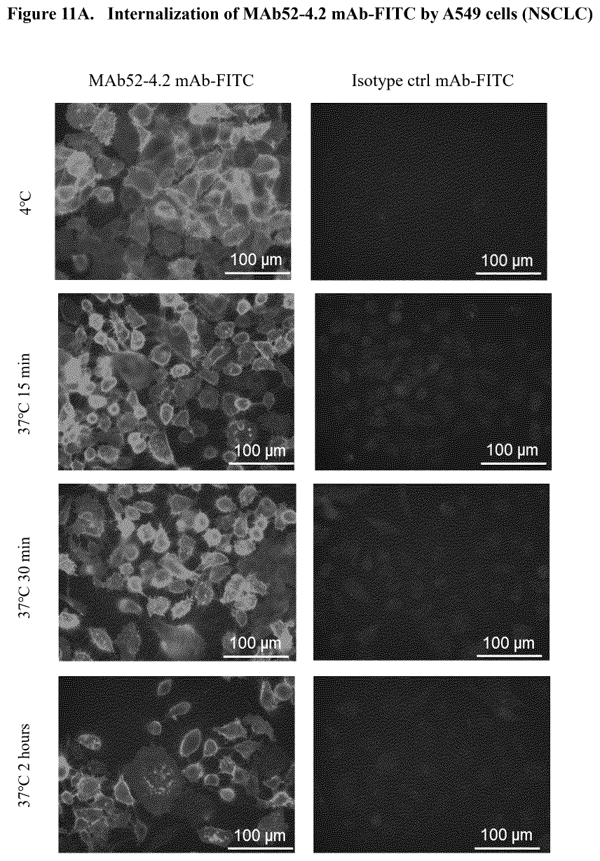

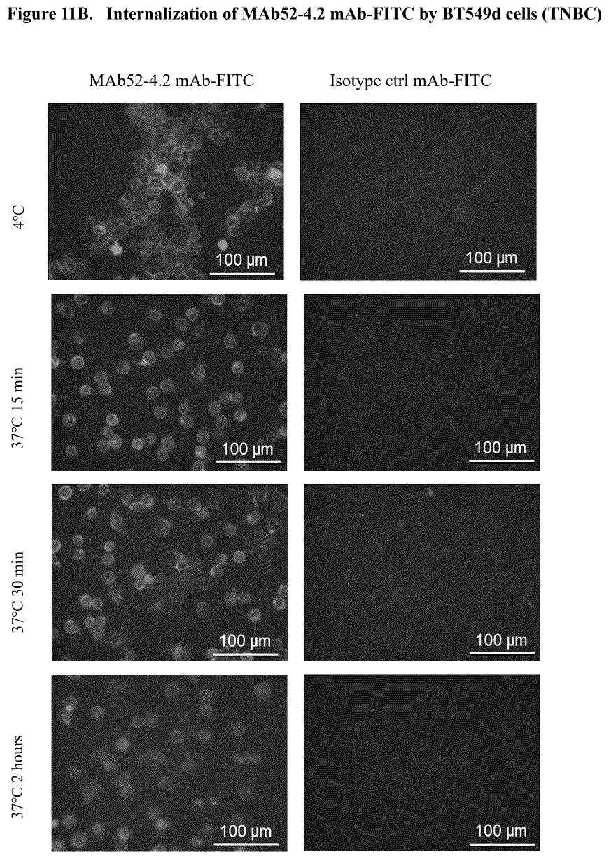

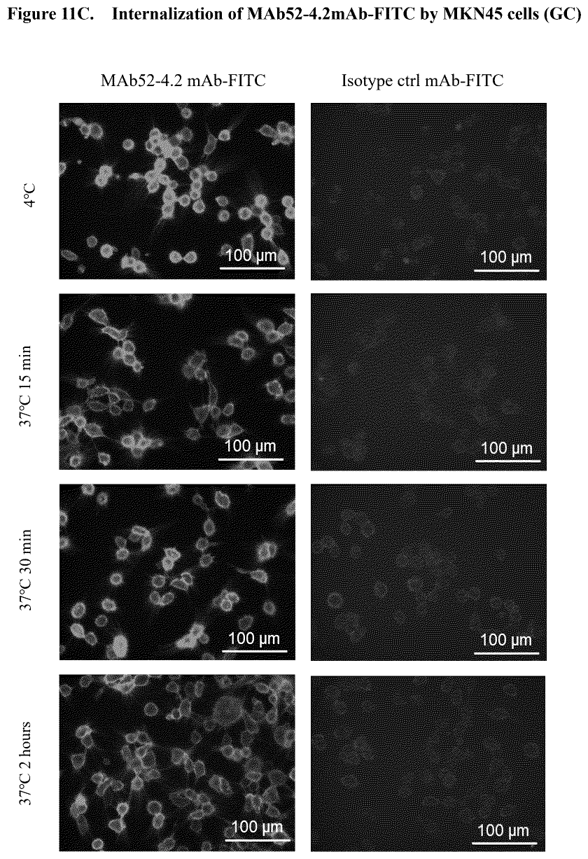

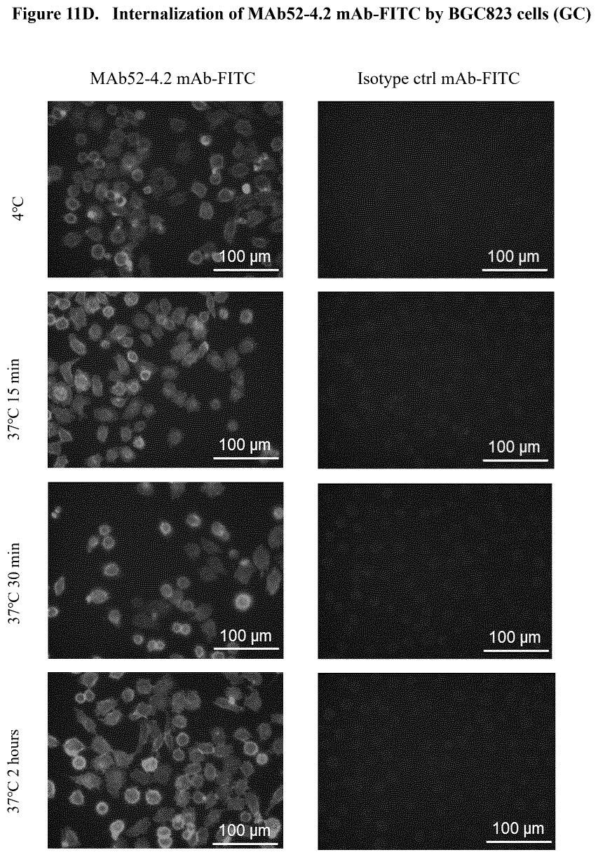

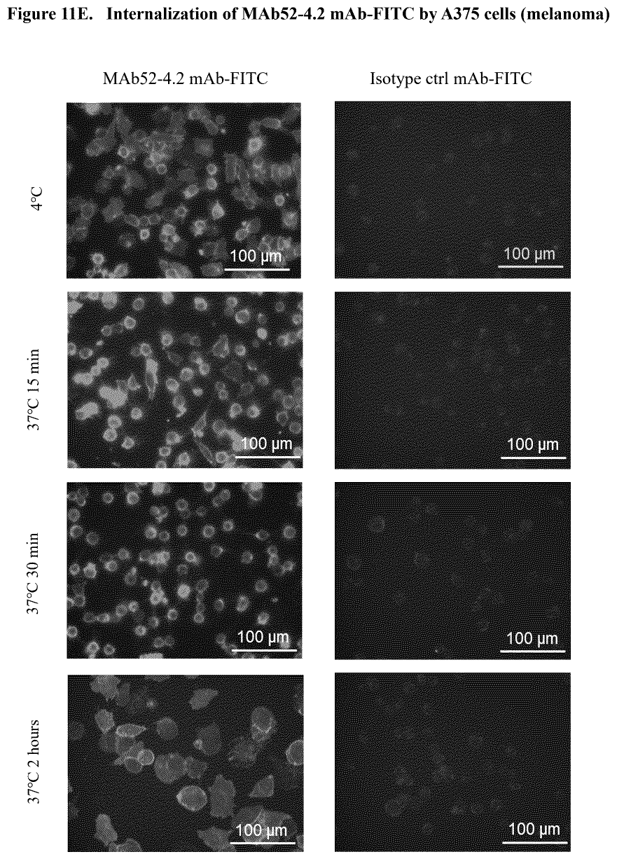

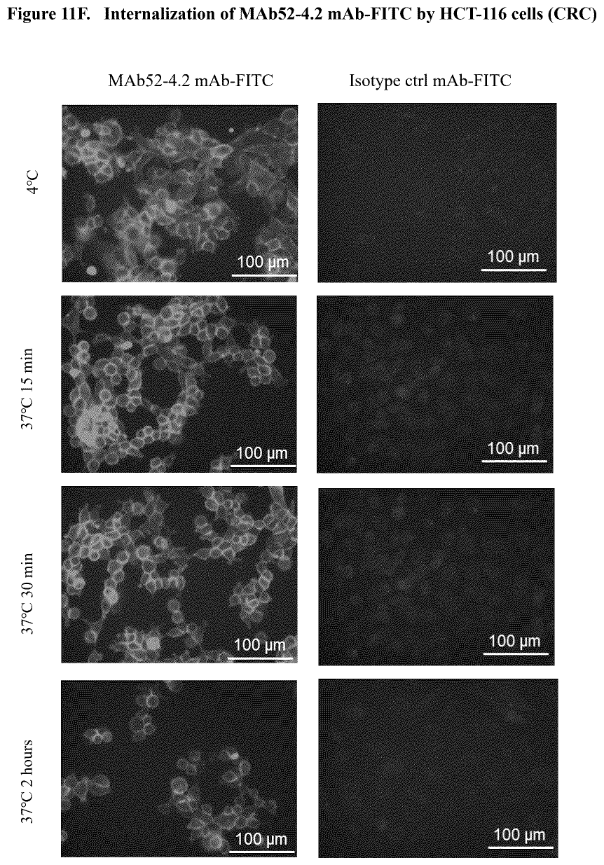

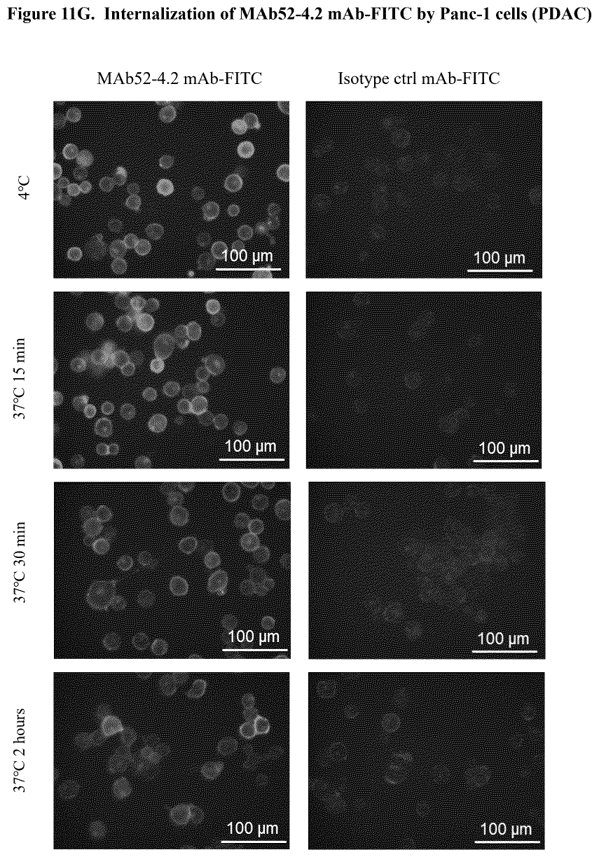

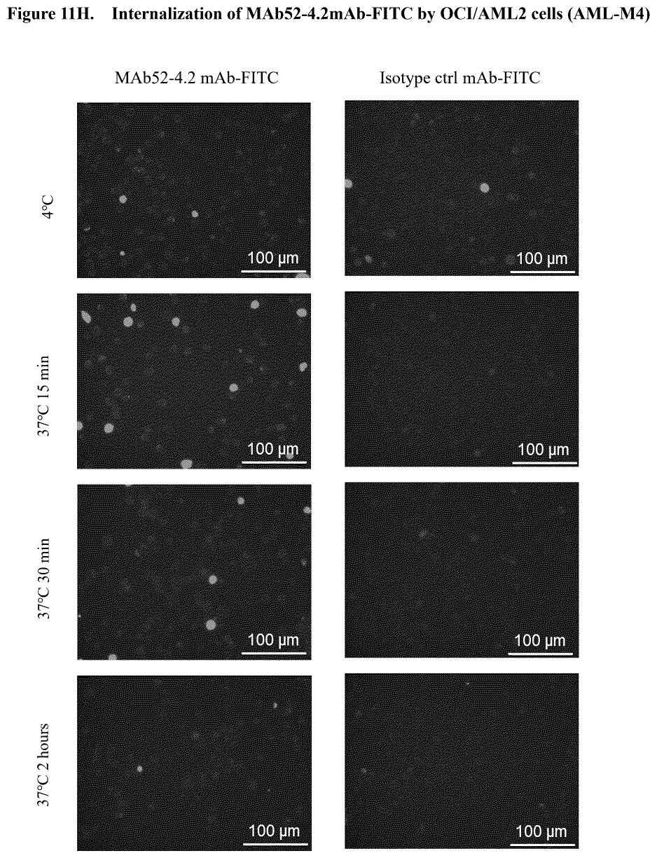

As expected, no binding was detected in any of the cancer cell lines treated with the isotype control mAb-FITC (FIGS. 11A to 11H, right panels). In contrast, MAb52-4.2mAb-FITC exhibited green fluorescence outlining the cell membrane after one hour of incubation at 4° C., confirming its binding to the target antigen on the cell surface (FIGS. 11A to 11G, left panels). The intensity of surface fluorescence correlated well with the cell staining results shown in FIG. 1A, with OCI/AML2 cells displaying much weaker fluorescence than the other cell lines. Notably, fluorescence accumulation in the cytoplasm was observed in A549 (FIG. 11A), MKN45 (FIG. 11C), BGC823 (GC; AffiGen, WI; FIG. 11D), and A375 (FIG. 11E) cells after one-hour at 4° C., suggesting partial internalization of the MAb52-4.2mAb-FITC and CD98hc complexes despite the reduced membrane fluidity at this temperature. Intracellular fluorescence intensified in these cells as incubation continued at 37° C., increasing progressively from 15 min to 2 hours. BT549d cells showed significant internalization within 15 min at 37° C., whereas no internalization was observed at 4° C. In contrast, no significant internalization was observed in HCT-116 (FIG. 11F) and OCI/AML2 (FIG. 11H) cells. Panc-1 cells exhibited strong self-fluorescence and were not assessable (FIG. 11G).

Variations in internalization efficiency may be influenced not only by antigen expression levels but also by intrinsic turnover rates of CD98hc and PTMs such as glycosylation, phosphorylation, and ubiquitination. These findings demonstrate efficient and robust internalization of MAb52-4.2 mAb in multiple cancer types, including A549, MKN45, BGC823, A375, and BT549d, highlighting its potential as an ADC candidate for multi-cancer treatment.

### Example 4

Molecular Cloning of the Light and Heavy Chain Genes Encoding MAb52-4.2 mAb and Expression of Chimeric Antibody (cAb)

### A. Cloning and Sequencing of the Light and Heavy Chain Genes by 5′-RACE

The cDNA sequences encoding the variable light (VL) and variable heavy (VH) regions of the MAb52-4.2 hybridoma were determined using 5′-Rapid Amplification of cDNA Ends (5′-RACE). Total RNA was extracted from the MAb52-4.2 hybridoma cells using the Quick-RNA™ Miniprep Kit (Zymo Research). Reverse transcription and first-strand cDNA synthesis were performed using the SMARTer RACE 5′/3′ Kit (Takara Bio USA, San Jose, CA). The resulting cDNAs was then subjected to 5′-RACE PCR amplification using the following reverse primers to pair with the common forward primer, Universal Primer A Mix (UPM), provided by the kit.

Light chain reverse primer:
(SEQ ID NO: 24)
5′-CTGCTCACTGGATGGTGGGAAGATGG-3′;
Heavy chain reverse primer:
(SEQ ID NO: 25)
5′-AGCTGGGAAGGTGTGCACAC-3′;

The amplified PCR products were cloned into the pRACE vector (Takara) and transformed into Stellar™ E. coli competent cells (Takara) under ampicillin selection. Ten E. coli colonies containing VL and VH DNA fragments were randomly selected for plasmid purification using Hi-Speed Mini Plasmid Kits (IBI Scientific, Dubuque, Iowa). The purified plasmids were subjected to Sanger sequencing (Molecular Cloning Laboratories, South San Francisco, CA), and all sequences were analyzed through BLAST (Basic Local Alignment Search Tool) search against public databases, including NCBI IgBlast (https://www.ncbi.nlm.nih.gov/igblast/), the international ImMunoGeneTics (IMGT) information System® (http://www.imgt.org/IMGT_vquest/), and Uniprot (https://www.uniprot.org/blast), for the novelty of the V domains. Consensus sequences of VL and VH of MAb52-4.2 mAb are shown in the SUMMARY section as SEQ ID NO: 1 and SEQ ID NO: 3, respectively. The encoded amino acid sequences are shown as SEQ ID NO: 2 and SEQ ID NO: 4, respectively.

B. Construction of Expression Plasmid for MAb52-4.2cAbm2

The VL and VH genes of MAb52-4.2 mAb were amplified via PCR using Q5® High-Fidelity DNA Polymerase (New England Biolabs) from the sequencing plasmids using the cloning primer pairs shown as SEQ ID NO: 26 to 29 below.

VL Cloning Forward primer:
(SEQ ID NO: 26)
5′-AGCACTAGTGCCGCCACCATGCATTTTCAAGTGCAGAT-3′;
VL Cloning Reverse primer:
(SEQ ID NO: 27)
5′-GAACACGCTGGGCGCCGCATCAGCCCGTCTTATTTC-3′;
VH Cloning Forward primer:
(SEQ ID NO: 28)
5′-AGCTCTAGAGCCGCCACCATGGGATGGAACTGTTTCATC-3′;
VH Cloning Reverse primer:
(SEQ ID NO: 29)
5′-GCCTTTGGTGCTAGCTGAGGAGACTGTGAGAGTG-3′.

The PCR products were gel-purified using the Monarch DNA Gel Extraction Kit (New England Biolabs) and cloned in-frame into the pCMV-hIgG1K-m2 vector with NEBuilder® HiFi DNA Assembly Cloning Kit (New England Biolabs), following the manufacturer's protocol. The pCMV-hIgG1K-m2 vector encodes the human kappa light chain constant region (CL) and the human IgG1 heavy chain constant region (CH1-CH3), under the control of the murine CMV promoter and the human CMV IE1 promoter, respectively. Additionally, each hIgG1 constant region contains two cysteine (Cys of C) point mutations, enabling rapid screening of ADC candidates via site-specific conjugation to various payloads and linkers. Upon in-frame fusion with the murine MAb52-4.2 VL and VH genes, the resulting pCMV-52-4.2cAbm2 plasmid facilitates the expression of a human-murine chimeric antibody (cAb) incorporating four free Cys residues, named MAb52-4.2cAbm2.

C. Expression of MAb52-4.2cAbm2

Following confirmation by Sanger sequencing, pCMV-52-4.2cAbm2 was electroporated into Dux-1S-HD cells, a CHO DG44-derived cell line capable of high-density growth in serum-free medium (SFM), such as CD DG44 medium (Thermo Fisher Scientific). The cells were then plated at a density of 2×10⁵ cells/well in selection medium in 96-well tissue culture microplates. After 2-3 weeks of incubation in a CO₂ incubator, stable cell lines secreting MAb52-4.2cAbm2 into the culture medium were screened by ELISA and FACS analysis to assess antibody expression and binding to cancer cell lines. The selected cell line was subsequently subcloned by limited dilution, adapted to SFM, and expanded for large-scale production of MAb52-4.2cAbm2. MAb52-4.2cAbm2 purified from the culture supernatants by Protein-A affinity chromatography was analyzed by SDS-PAGE to ensure its integrity and purity.

### Example 5

Characterization of MAb52-4.2cAbm2 for Binding to Cancer Cell Lines and rhCD98hc-ECD-his Protein
A. FACS Analysis of MAb52-4.2cAbm2 Binding to Various Cancer Cell Lines

FACS analysis was conducted to assess the interaction between MAb52-4.2cAbm2 and various cancer cell lines, following the protocol outlined in Example 1. The only modification was the replacement of the anti-mouse IgG secondary antibody with Fluorescein AffiniPure™ Goat Anti-Human IgG, Fcγ fragment specific, to detect surface-bound cAbm2. An isotype control cAbm2, consisting of human IgG1 CH1-CH3 and human Kappa CL, served as the negative control.

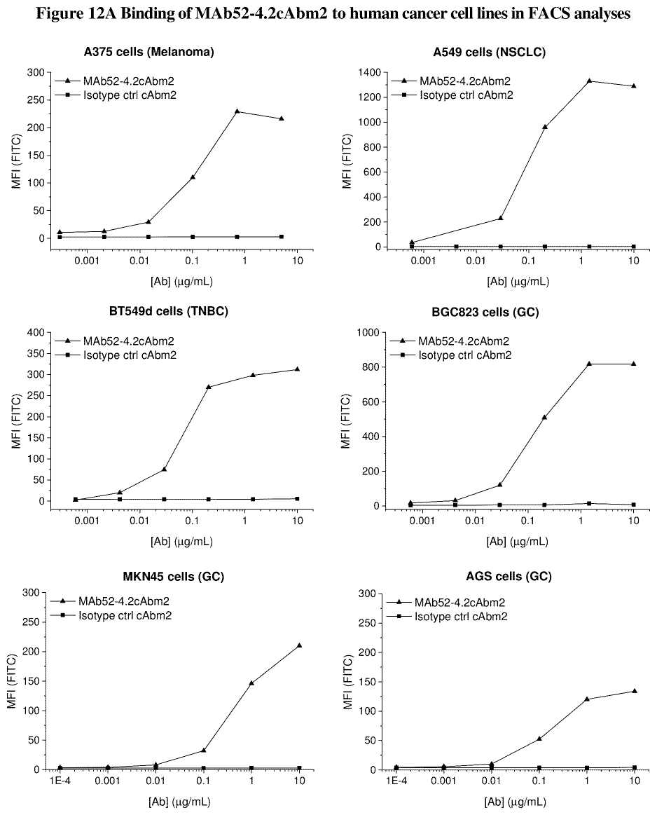

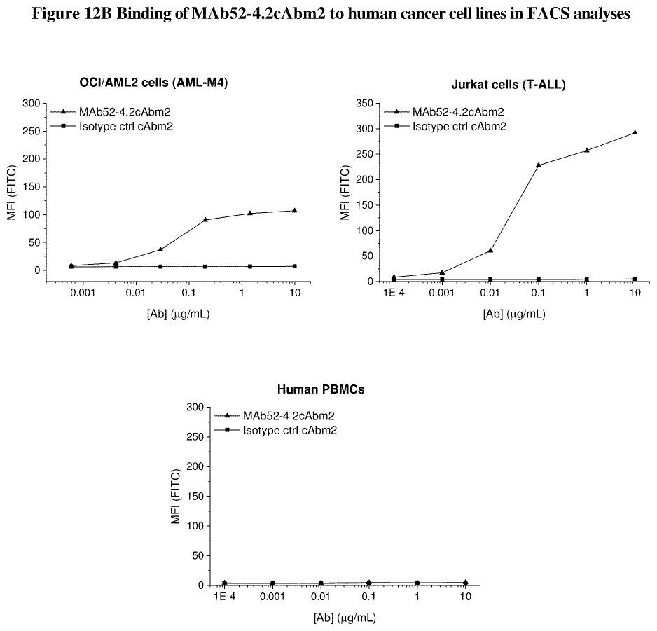

As shown in FIG. 12, MAb52-4.2cAbm2 exhibited dose-dependent binding to all tested solid tumor cell lines, with a binding pattern and EC₅₀ comparable to its mAb counterpart (see FIGS. 1A and 1B). These data suggest that replacement of murine constant regions with human constant regions with the Cys point mutations, did not impact the binding specificity of the chimeric antibody.

B. Binding Kinetics and Affinity Between MAb52-4.2cAbm2 and rhCD98hc-ECD-his

The interaction between MAb52-4.2cAbm2 and rhCD98hc-ECD-His was analyzed using the Octet® QK^{e} System equipped with Anti-Human IgG Fc Capture (AHC) Biosensors (Sartorius) following the protocol described in Example 2C. One hundred nanomolar (100 nM) MAb52-4.2cAbm2 was immobilized onto the AHC biosensors for 10 min, followed by a 5-min wash, a 15-min association phase with rhCD98hc-ECD-His, and a 30-min dissociation phase. The assay was repeated multiple times with rhCD98hc-ECD-His concentrations ranging from 200 nM to 12.5 nM, using 2-fold serial dilutions. A 1:1 binding model was applied to fit the binding curves.

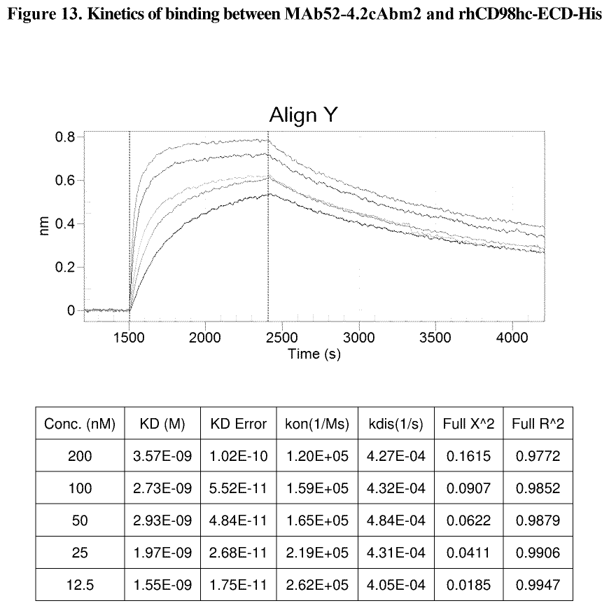

As shown in FIG. 13, MAb52-4.2cAbm2 exhibited a fast association rate and a moderate dissociation rate, with a calculated K_{D} of approximately 2.6×10⁻⁹ M that was similar to the murine mAb counterpart (2.2×10⁻⁹ M; FIG. 7).

### Example 6

Preparation and Characterization of MAb52-4.2cAbm2-MMAE ADC
A. Site-Specific Conjugation of MAb52-4.2cAbm2 with Vc-MMAE

MAb52-4.2 mAb demonstrated strong cancer-specificity and internalization capability in multiple cancer cell lines, making it a promising candidate for ADC therapy. A preliminary study using monomethyl auristatin E (MMAE)-conjugated MAb52-4.2 mAb to the endogenous Cys residues showed promising inhibitory effect on cancer cell proliferation (data not shown). To further explore its potential as an ADC candidate, MAb52-4.2cAbm2 and the non-cell binding isotype control cAbm2, which were engineered with two Cys point mutations in each IgG1 heavy chain constant region for site-specific conjugation using the maleimide-valine-citrulline-PAB-MMAE (VcMMAE) linker-payload reagent (BroadPharma, San Diego, CA). VcMMAE consists of a lysosomal-cleavable dipeptide linker (Vc), a para-aminobenzyloxycarbonyl (PABC) spacer, an anti-tubulin toxin MMAE, and a thiol-reactive maleimidocaproyl (MC) group that specifically reacts with the free thiol group in cysteines. VcMMAE has been demonstrated to be an effective payload-linker in ADC in treating many types of cancer. It is a key component in FDA-approved ADCs, including Brentuximab vedotin (Adcetris®), Polatuzumab vedotin (Polivy®), Enfortumab vedotin (Padcev®), and Tisotumab vedotin (Tivdak®) (Gogia et al. Cancers, 2023; Liu et al. Mol Cancer 2024). Consequently, we employed VcMMAE in our ADC platform to quickly screen mAbs as ADC candidates.

Site-specific conjugation was performed following the method described by Junutula et al. (Nat Biotechnol. 26:925-932, 2008). Briefly, MAb52-4.2cAbm2 was reduced with 1 mM dithiothreitol (DTT) to remove cysteine or glutathione adducts from the culture medium. The antibody was then refolded into its native structure in the presence of 0.4 mM dehydroascorbic acid (DHAA; MilliporeSigma), and unfolded antibody light and heavy chains were removed using an Amicon® Ultra Centrifugal Filter (50 kDa MWCO; MilliporeSigma). For conjugation, VcMMAE and MAb52-4.2cAbm2 were mixed at a 10:1 molar ratio with gentle agitation at RT for one hour, followed by size exclusion chromatography to remove unbound VcMMAE and potential aggregates. This site-specific conjugation protocol results in ADCs with an average drug-to-antibody ratio (DAR) of approximately 4 (DAR4).

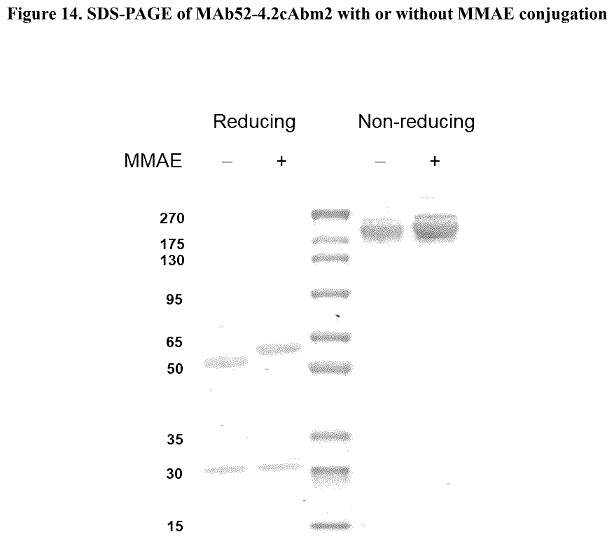

The concentration of MAb52-4.2cAbm2-MMAE was measured using NanoDrop™ 2000 at 280 nm and confirmed with the Pierce™ Bradford Protein Assay Kit (Thermo Fisher Scientific). SDS-PAGE analysis under reducing conditions revealed distinct light and heavy chain bands, with no visible aggregation or degradation (FIG. 14). As expected, the MMAE-conjugated heavy chain band migrated higher than the unconjugated heavy chain band, indicating successful conjugation. Non-reducing SDS-PAGE showed a minor, but acceptable, fraction of aggregates.

B. In Vitro Inhibitory Effects of MAb52-4.2cAbm2-MMAE on Cancer Cell Proliferation

The effect of MAb52-4.2cAbm2-MMAE on cancer cell proliferation was evaluated using cell line-based cytotoxic assays as previously described (see U.S. Pat. No. 11,939,391B2). Briefly, cancer cell lines cultured in DMEM with 10% (v/v) FBS were seeded at 3-5×10³ cells/well, depending on cell type, in triplicate in 96-well tissue culture microplates. Cells were treated with MAb52-4.2cAbm2-MMAE or the non-cell binding isotype control cAbm2-MMAE at serial dilutions with concentrations starting from 2 to 20 μg/mL (13.3~133 nM). After four days of incubation at 37° C. under a 5% CO₂ humidified atmosphere, cell viability was assessed by adding 1/10 volume of the Cell Counting Kit-8 (CCK-8) solution (Dojindo Molecular Technologies, Rockville, MD) and incubating for four hours at 37° C., followed by measuring the absorbance at 450 nm (A450).

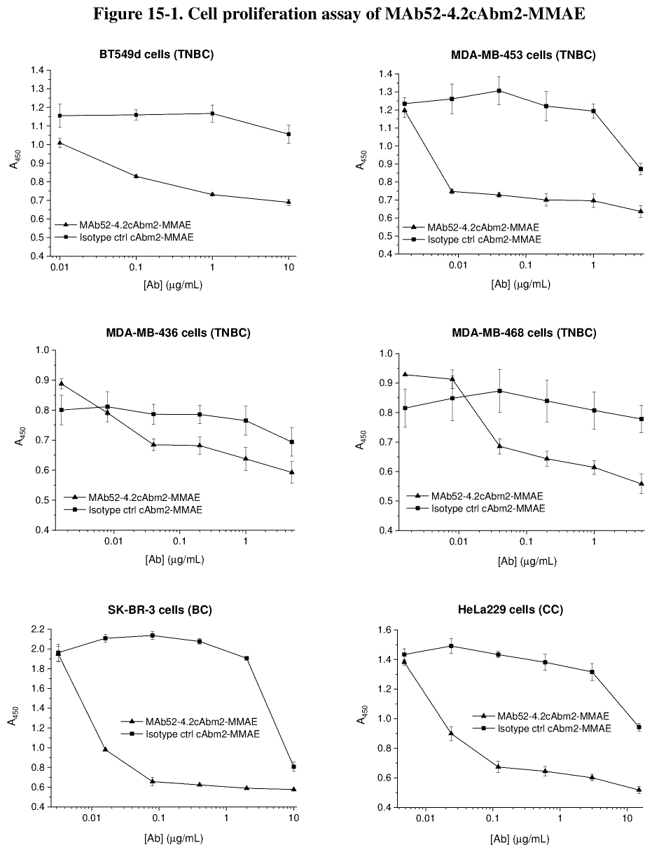

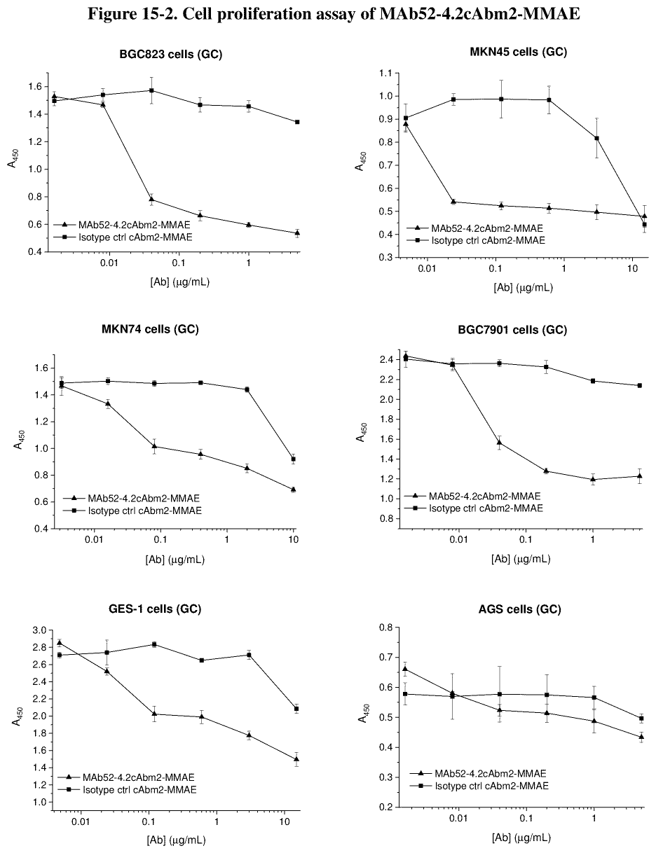

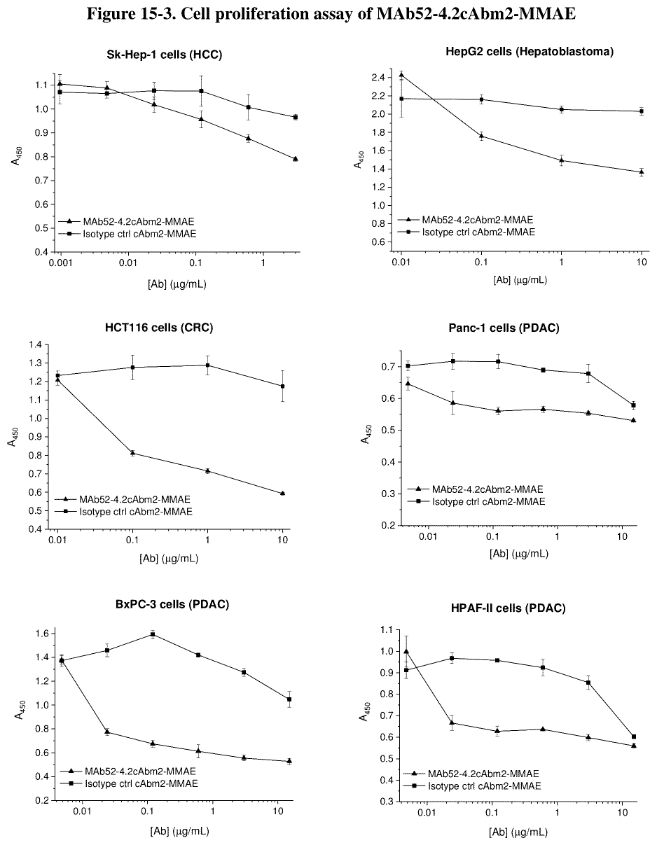

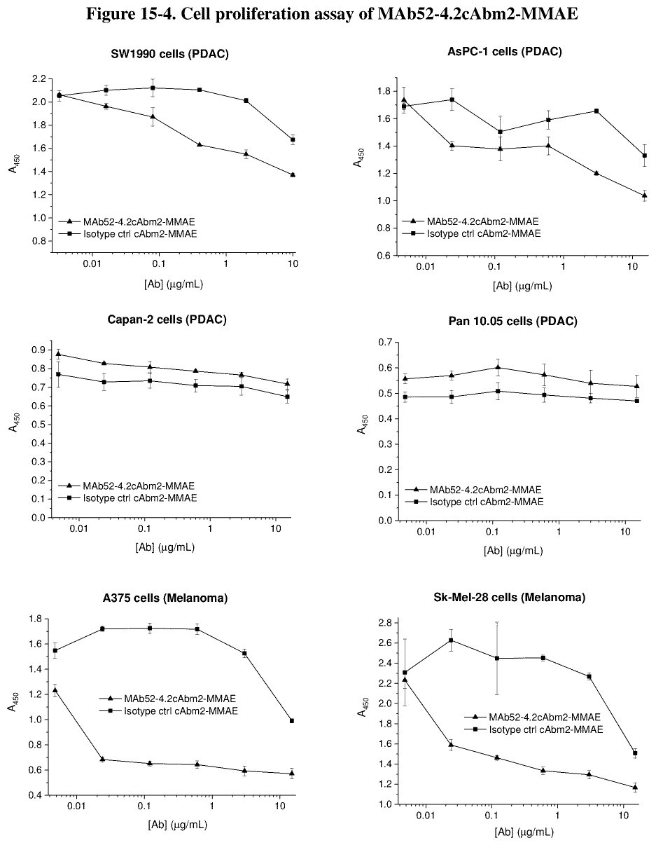

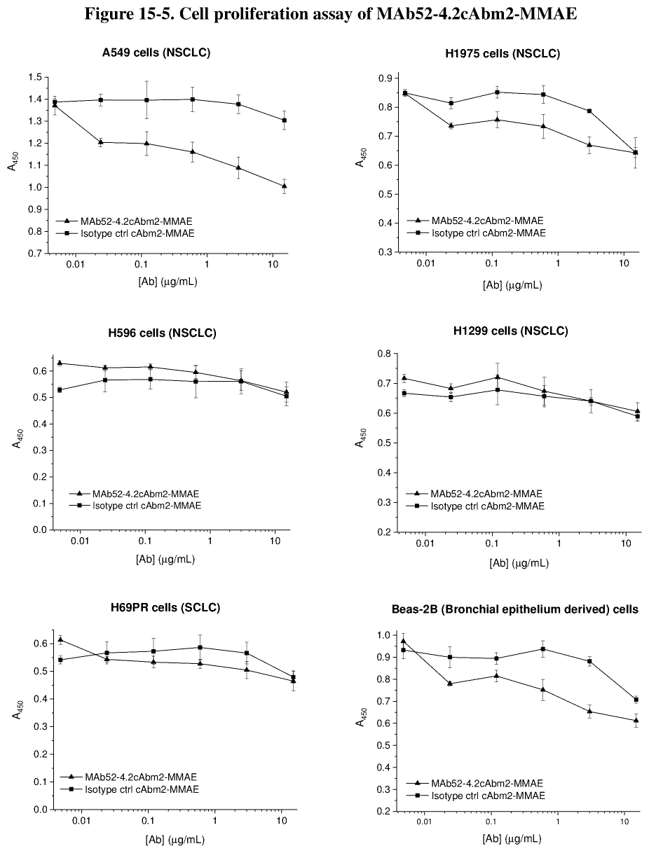

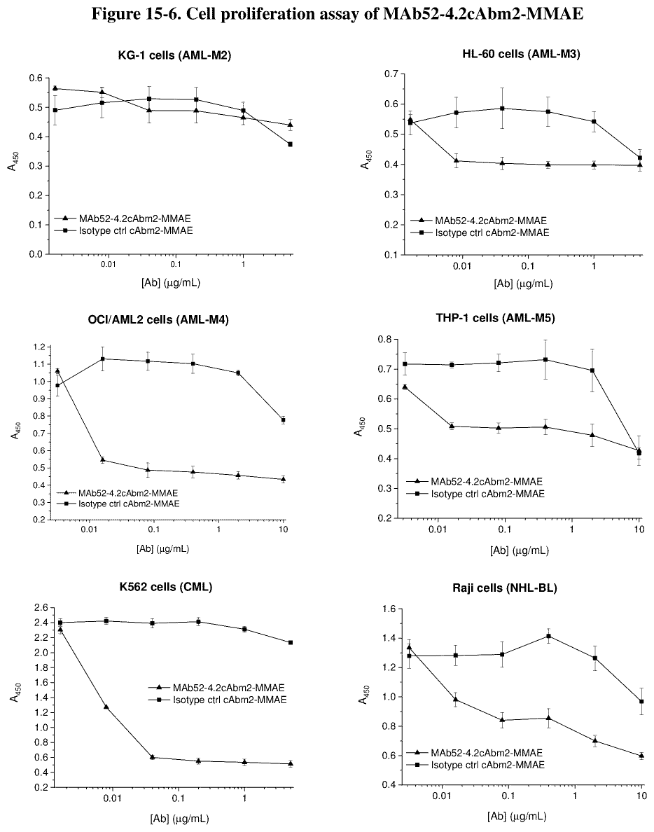

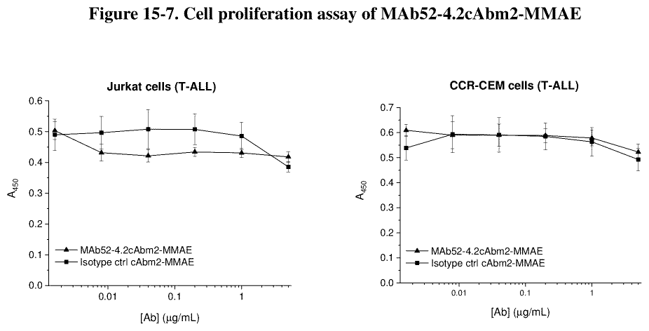

As shown in FIG. 15, exposure to MAb52-4.2cAbm2-MMAE led to a significant, dose-dependent reduction in cell proliferation across most tested cell lines, including TNBC, GC, CRC, CC, PDAC, melanoma, AML, and CLL. In contrast, most NSCLC and some PDAC cell lines, along with two liver cancer cell lines and two ALL cell lines, did not respond to MAb52-4.2cAbm2-MMAE. These findings suggest that MAb52-4.2cAbm2-MMAE effectively suppresses cancer cell proliferation, likely due to its strong binding affinity, efficient internalization, and subsequent lysosomal release of MMAE, which disrupts cell division by binding to tubulin dimers and interfering with the microtubule network, leading to mitotic arrest and cell death.

### Example 7

In Vivo Tumor Inhibition Study of MAb52-4.2cAbm2-MMAE on Cancer Cell-Derived Xenograft (CDX) Mouse Models

Six cell-derived xenograft (CDX) mouse models, including BT549d (TNBC), BGC823 (GC), MKN45 (GC), HCT-116 (CRC), A549 (NSCLC), and A375 (melanoma), were utilized to evaluate the antitumor effects of a single-dose treatment with MAb52-4.2cAbm2-MMAE (DAR≈4). For each model, 2×10⁶ to 5×10⁶ cancer cells, suspended in 0.1 ml of PBS, were subcutaneously (s.c.) inoculated into the flanks of 6-week-old male athymic nude mice (Charles River Laboratories, Wilmington, MA). Mice were randomized into five per treatment group when the majority of tumors reached 100 mm³ in size (Day 0).

Mice received intraperitoneal (i.p.) injections of either MAb52-4.2cAbm2-MMAE or isotype control cAbm2-MMAE (DAR≈4) at doses of 4 mg/kg, 7 mg/kg, or 10 mg/kg. In some studies, a vehicle-only control group was treated with 0.1 ml of PBS to assess potential off-target effect of the ADCs. Tumor size and body weight were monitored every three to four days over four to five weeks, depending on tumor aggressiveness. Tumor volumes were calculated using the formula: Volume=½× (width)²× length. Animals were euthanized if tumor volume exceeded 2,000 mm³ or if the tumor diameter surpassed 20 mm.

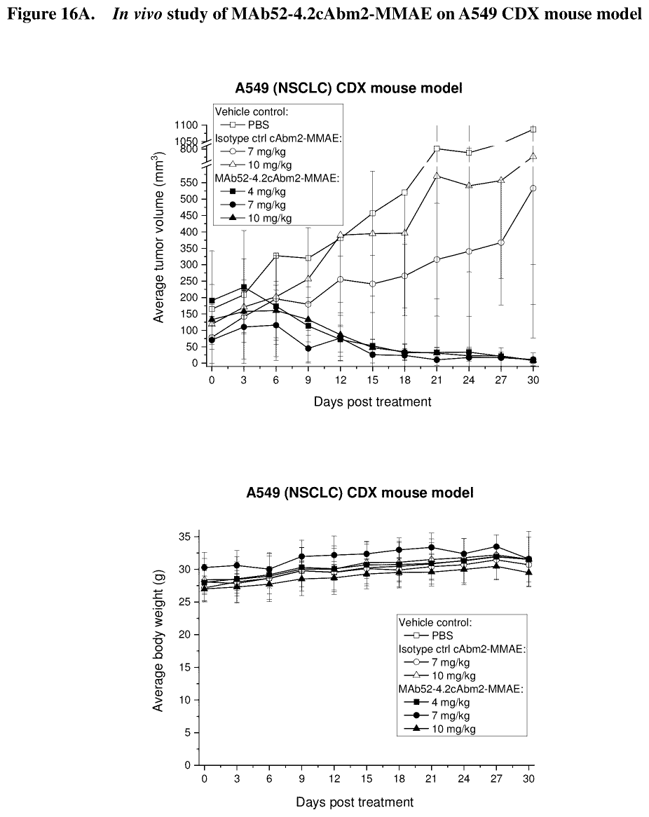

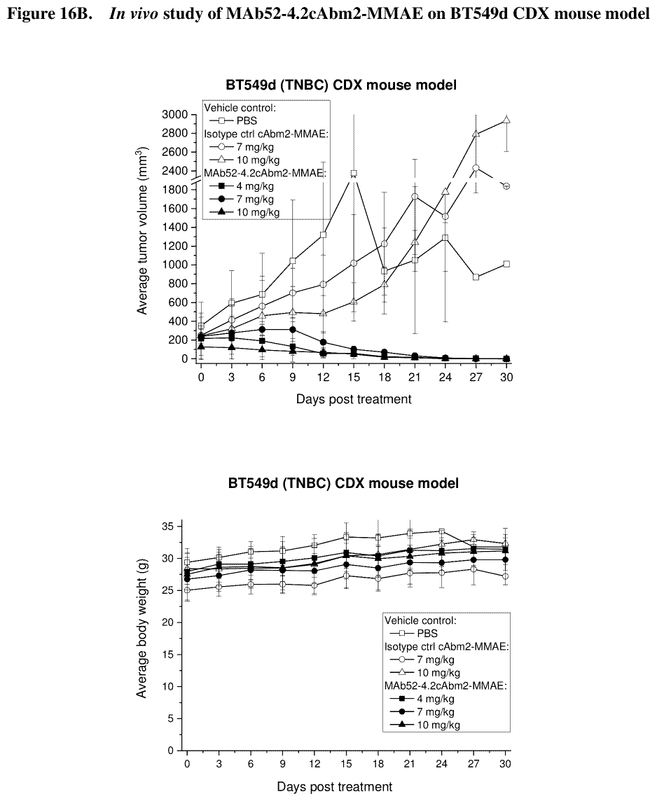

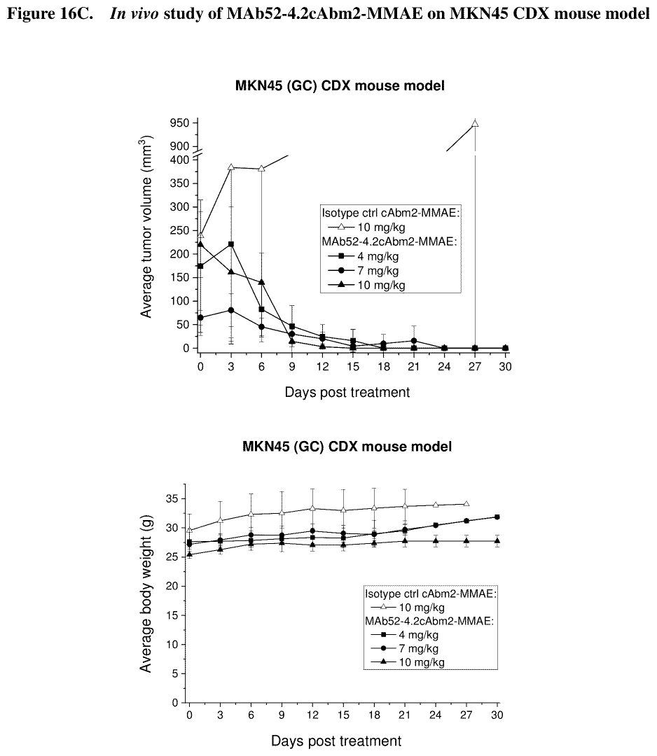

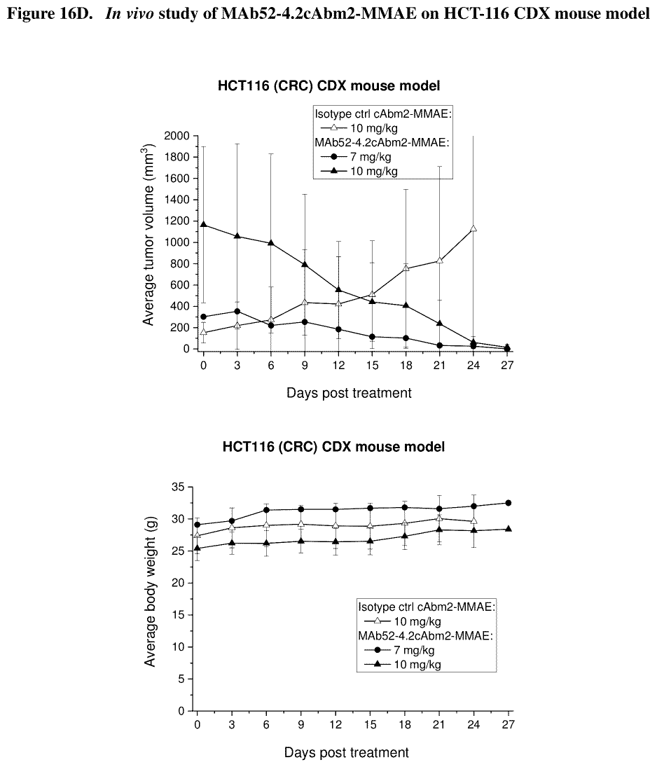

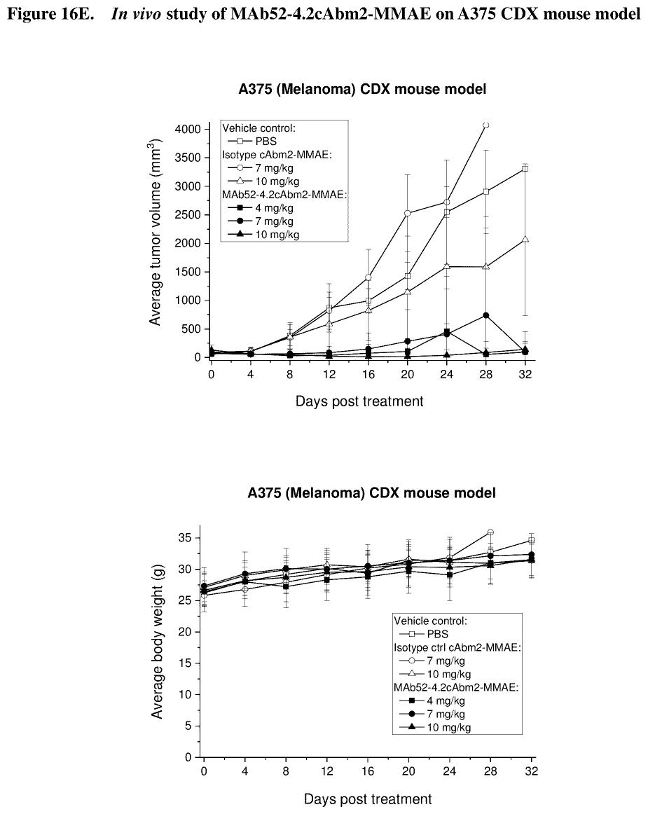

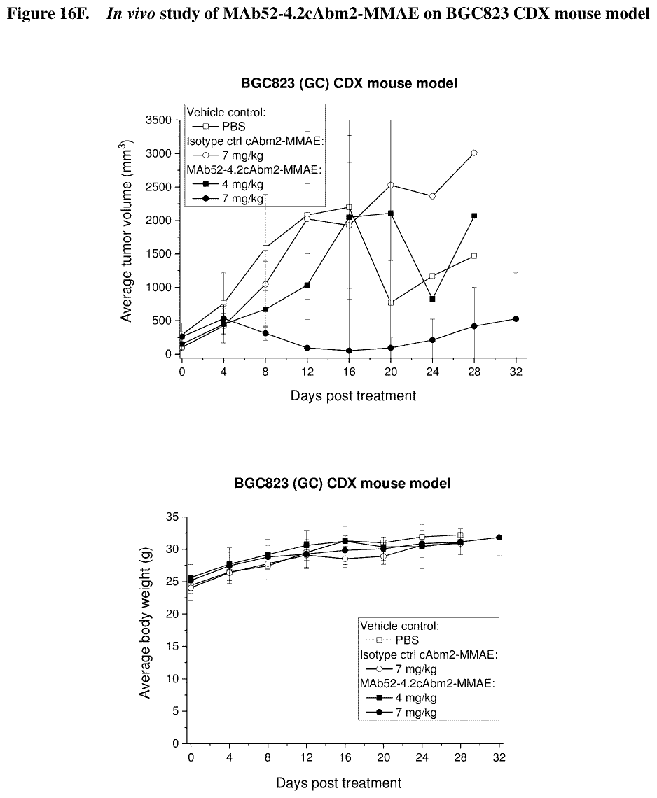

FIGS. 16A to 16F illustrate the average tumor volume and body weight in each treatment group. Compared to the vehicle-treated and isotype control cAbm2-MMAE-treated groups, MAb52-4.2cAbm2-MMAE significantly reduced tumor volumes in the A549, BT549d, and MKN45 CDX models across all three tested doses (FIGS. 16A to 16C). In these models, tumor nodules halted growth within six to eight days post-treatment, with most tumors in the high- and medium-dose groups continuing to regress until became undetectable, suggesting a strong bystander effect of the MAb52-4.2 ADC.

In contrast, MAb52-4.2cAbm2-MMAE was less effective against A375 tumors as it reduced tumor growth rate but failed to achieve full suppression (FIG. 16E), indicating that multiple-dose treatment may be necessary for complete tumor eradication. BGC823 tumors did not respond to 4 mg/kg dose and exhibited only partial regression at 7 mg/kg, despite promising antibody binding and internalization data (FIG. 16F). Preliminary results from the HCT-116 tumor model showed encouraging tumor regression in cases where tumors had reached sizes of 1400 to 1800 mm³ prior to treatment (FIG. 16D). However, due to the small sample size and the slow progression of tumors in the isotype control group, a definitive conclusion could not be drawn.

Nevertheless, the anti-tumor effect of a single-dose MAb52-4.2cAbm2-MMAE administration persisted for more than three to four weeks across multiple models. It is anticipated that multiple-dose regimens could achieve complete tumor control in these CDX models.

In the A549, BT549d, A375, and BGC823 CDX models, the isotype control cAbm2-MMAE exhibited a slightly reduced tumor growth rate compared to the vehicle-treated group (FIGS. 16A, 16B, 16E, and 16F), suggesting a minor non-specific effect, potentially due to the instability of the cleavable Val-Cit-PAB linker in vivo. While this difference was not significant, it highlights the need for a more stable linker in future ADC development.

As observed in the in vitro assays, the varying responses of different tumor models to MAb52-4.2cAbm2-MMAE may be influenced by several factors, including differences in surface expression of CD98hc, internalization efficiency of the ADC-CD98hc complex, and cellular sensitivity to MMAE. Additionally, for in vivo studies, ADC penetration through the tumor microenvironment (TME) could further impact therapeutic efficacy.

No adverse effects, such as weight loss, fever, lethargy, vomiting, diarrhea, bleeding, or loss of appetite, were observed in any of the MMAE-conjugated antibody-treated groups.

### Example 8

### Humanization of MAb52-4.2 mAb and Verification of Target Binding for MAb52-4.2HuAbm2s

### A. Engineering MAb52-4.2 HuAbs Via CDR Grafting and Strategic Back-Mutations

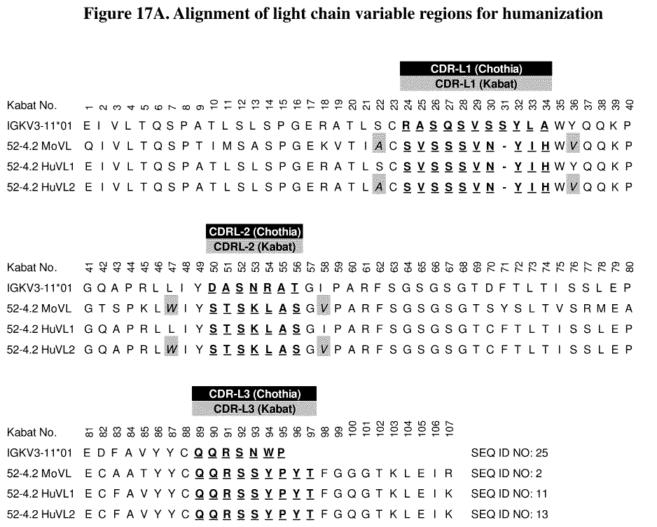

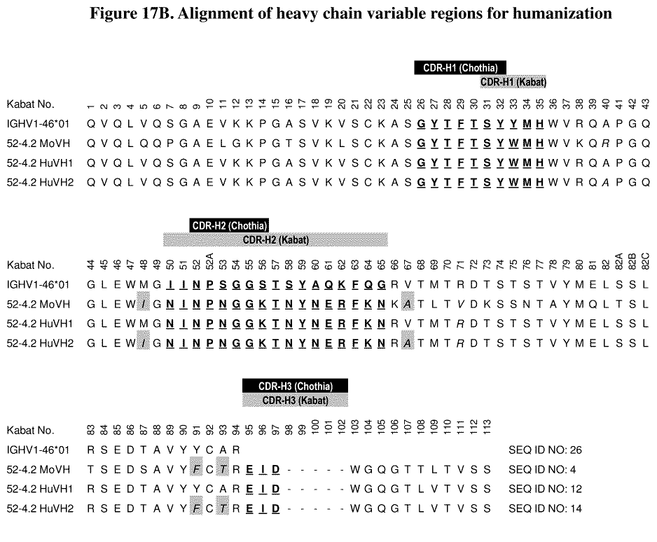

The VL (SEQ ID NO: 2) and VH (SEQ ID NO: 4) regions of murine MAb52-4.2 mAb were compared against human antibody germline sequences available in public databases. The optimal human germline sequences selected as the templates for humanization are shown in FIGS. 17A and 17B. Specifically, the VL frameworks (FRs) of human IGKV3-11*01 (SEQ ID NO: 25) and the VH FRs from human IGHV1-46*01 (SEQ ID NO: 26) were chosen to replace the corresponding murine FRs of MAb52-4.2 mAb. The joint (J) region sequences selected were from human IGKJ2 (YTFGQGTKLEIK, SEQ ID NO: 27) and IGHJ4 (WGQGTLVTVSS, SEQ ID NO: 28) sequences. The sequence identities between murine and human FRs are relatively high, ranging from 61% to 69% for VL or 56% to 80% for VH. Therefore, only two sets of HuAb sequences were designed, one comprising HuVL1 (SEQ ID NOs: 11) and HuVH1 (SEQ ID NOs: 12) without any back mutation, while the other containing HuVL2 (SEQ ID NOs: 13) and HuVH2 (SEQ ID NOs: 14) with back mutations.

The cDNA fragments encoding these humanized VL and VH sequences were synthesized and annealed respectively with the human light and heavy chain constant regions encoded by the pCMV-hIgG1K-m2 expression vector following the protocol described in Example 4B. The expression plasmids also contained two free cysteine point mutations in each of the Fc region to allow site-specific conjugation via disulfide bonds. Sanger sequencing confirmed the antibody coding regions in the plasmids, which were then transfected into HEK293FT cells (Thermo Fisher Scientific) using FectoPro™ Transfection Reagent (PolyPlus Illkirch, France) according to the manufacturer's protocol. Cell cultures were maintained in 30 mL of SFM containing DMEM with 1.0% (w/v) BSA. Three days post-transfection, the humanized antibodies secreted in the culture supernatants was quantified using a capture ELISA assay immobilized with AffiniPure™ Goat Anti-Human IgG, Fcγ Fragment Specific polyclonal antibody (Jackson ImmunoResearch laboratories).

B. Binding of Humanized MAb52-4.2HuAbm2 with Cancer Cell Lines

Transiently expressed antibodies in the culture supernatants were assessed for cell binding using FACS analysis with A375 and BGC823 cell lines. The result showed that the HuVL1 and HuVH1 combination without framework back mutations failed to stain any of the cells, whereas the HuVL2 and HuVH2 combination exhibited binding capacity to cancer cells comparable to MAb52-4.2cAbm2 (data not shown). This finding indicates that the selected residues for back mutations were essential for target recognition. Consequently, the humanized version consisting HuVL2 and HuVH2 with back mutations, designated as MAb52-4.2HuAbm2, was used for further evaluations.

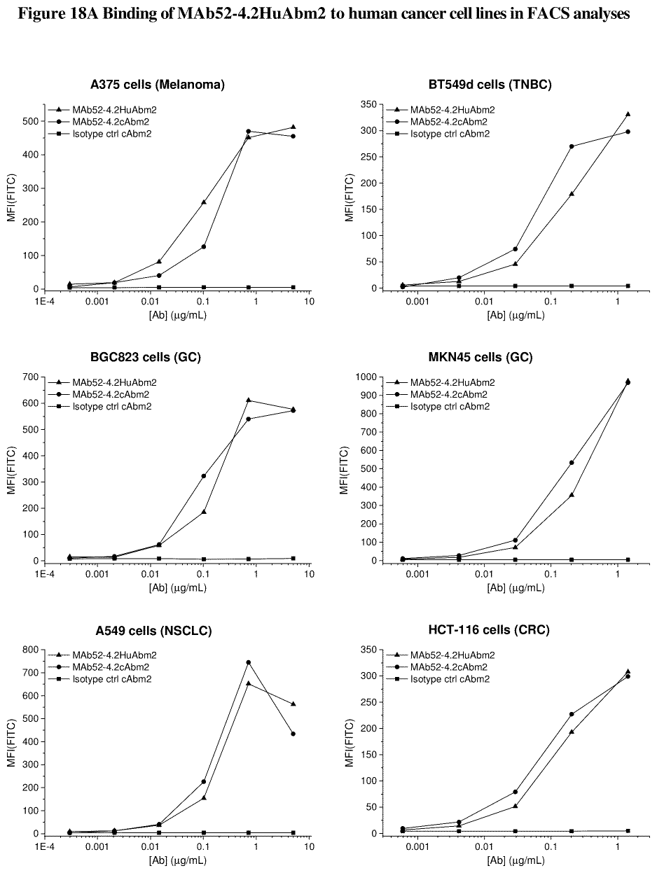

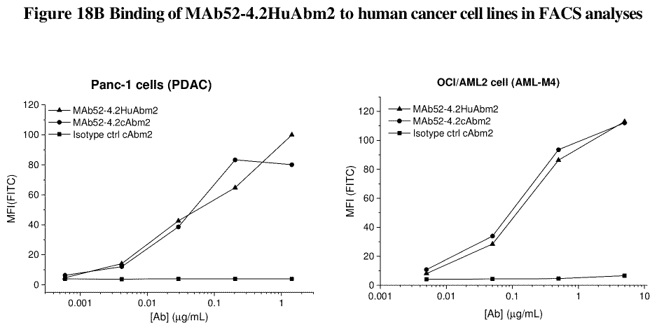

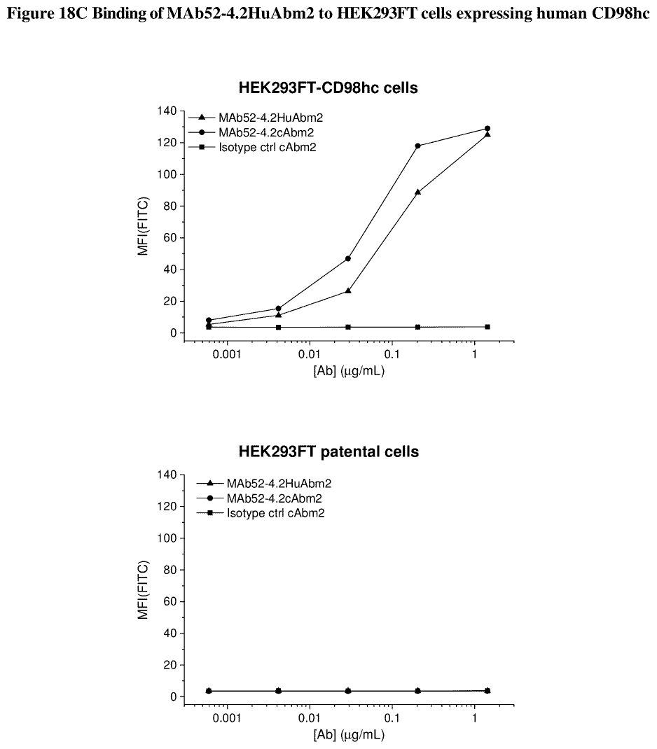

Cell staining of eight representative human cancer cell lines confirmed a dose-dependent binding pattern for MAb52-4.2HuAbm2, comparable to MAb52-4.2cAbm2 (FIG. 18A). Furthermore, both MAb52-4.2HuAbm2 and MAb52-4.2cAbm2 demonstrated binding to HEK293FT cells expressing CD98hc, while no binding was observed in the parental HEK293FT cells (FIG. 18B), further validating target specificity.

C. Competitive ELISA Assay of MAb52-4.2cAbm2 and MAb52-4.2HuAbm2 in Antigen Binding

A competitive ELISA assay was performed to determine whether MAb52-4.2cAbm2 and MAb52-4.2HuAbm2 retain the ability to recognize the same epitope on rhSLC3A2&SLC7A11-VLP as MAb52-4.2 mAb. In this assay, MAb52-4.2 mAb was conjugated with HRP using Lightning-Link® HRP Conjugation Kit as described in Example 2A. The titer of MAb52-4.2mAb-HRP was determined in an ELISA assay using plates coated with 30 ng/well of rhSLC3A2/SLC7A11-VLP. Subsequently, unconjugated MAb52-4.2 mAb, MAb52-4.2cAbm2, MAb52-4.2HuAbm2, or the isotype control cAbm2 were serially diluted in three folds, mixed with 1:10,000 diluted MAb52-4.2mAb-HRP, and incubated with immobilized rhSLC3A2/SLC7A11-VLP for one hour. After thorough wash, TMB substrate was added to detect binding signals between MAb52-4.2mAb-HRP and the antigen.

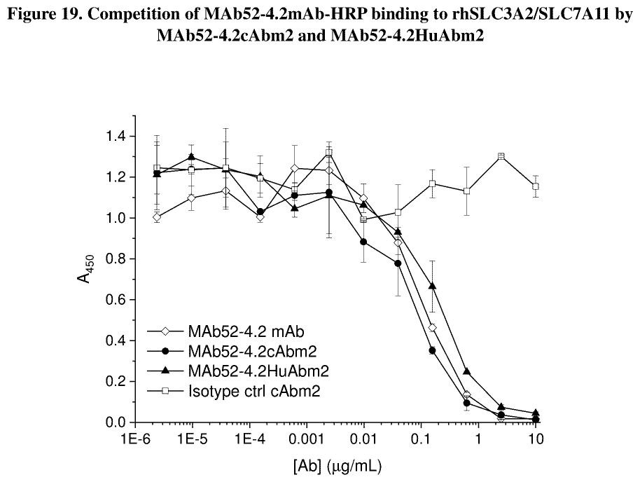

As shown in FIG. 19, the isotype control cAbm2, which targets an irrelevant antigen, served as the baseline for MAb52-4.2mAb-HRP and rhSLC3A2&SLC7A11-VLP binding. Both MAb52-4.2cAbm2 and MAb52-4.2HuAbm2 exhibited a dose-dependent reduction in binding signals similar to MAb52-4.2 mAb, with nearly overlapping curves. This indicates that they recognize overlapping or identical epitopes on rhSLC3A2/SLC7A11-VLP and possess comparable apparent affinities. Furthermore, the findings suggest that substituting the murine antibody constant regions with human heavy chain constant regions containing Cys point mutations did not affect epitope recognition. However, additional structural and kinetic analyses are necessary to confirm complete epitope identity and binding affinity equivalence.

D. Binding Kinetics and Affinity Between MAb52-4.2HuAbm2 and rhCD98hc-ECD-his

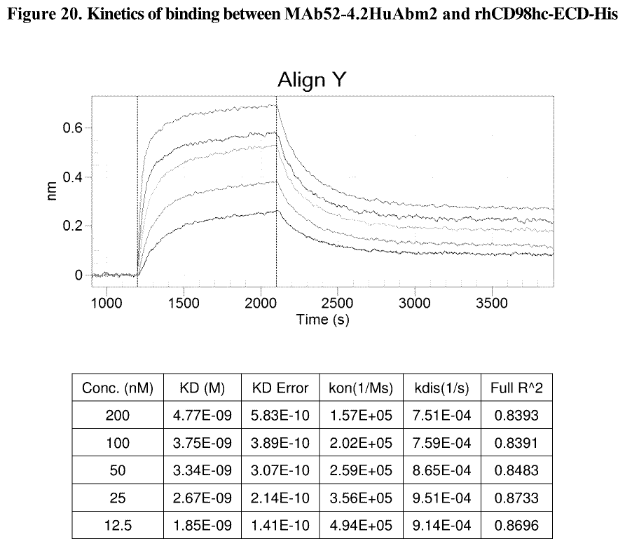

The binding kinetics and affinity between MAb52-4.2HuAbm2 and rhCD98hc-ECD-His were determined using the same protocol as for MAb52-4.2cAbm2. MAb52-4.2HuAbm2 demonstrated a binding affinity constant (K_{D}) of 3.3×10⁻⁹ M (FIG. 20), which is closely comparable to MAb52-4.2 mAb (2.2×10⁻⁹ M) and MAb52-4.2cAbm2 (2.6×10⁻⁹ M). These results indicate that replacing the murine FR sequences with human FRs from IGKV3-11*01 (SEQ ID NO: 25) and IGHV1-46*01 (SEQ ID NO: 26), along with the designed back mutations, did not compromise the antibody's binding affinity to its target.

E. Internalization Assay of MAb52-4.2HuAbm2—CF488 with Various Cancer Cell Lines

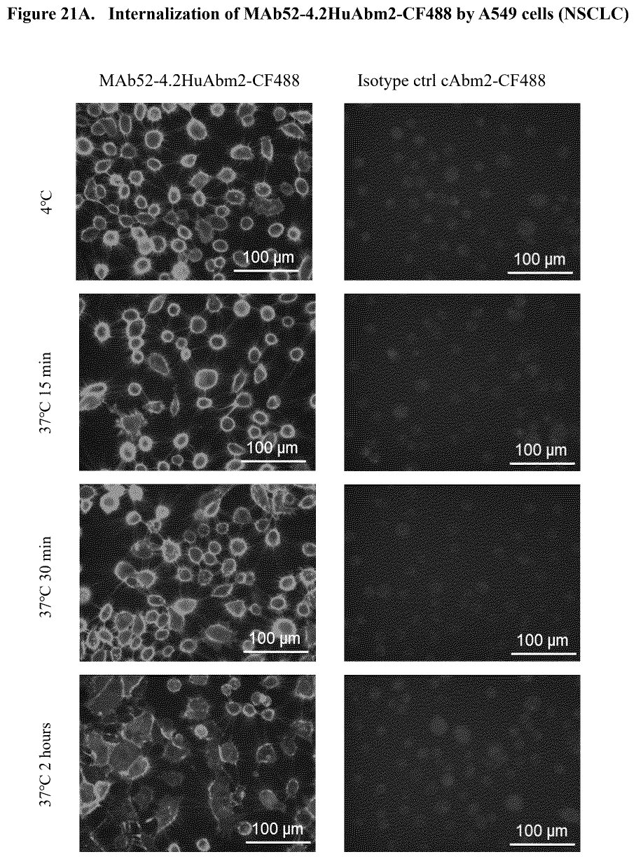

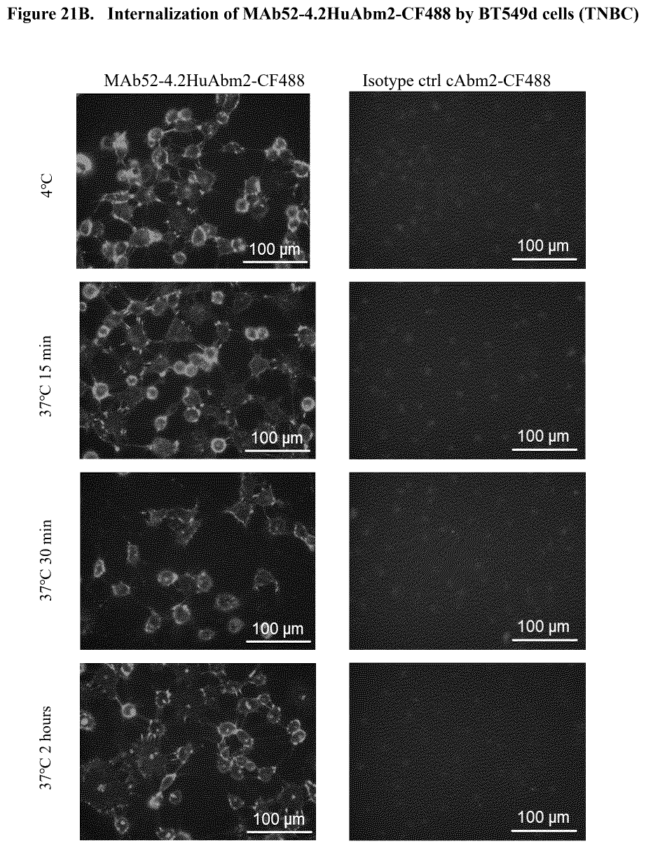

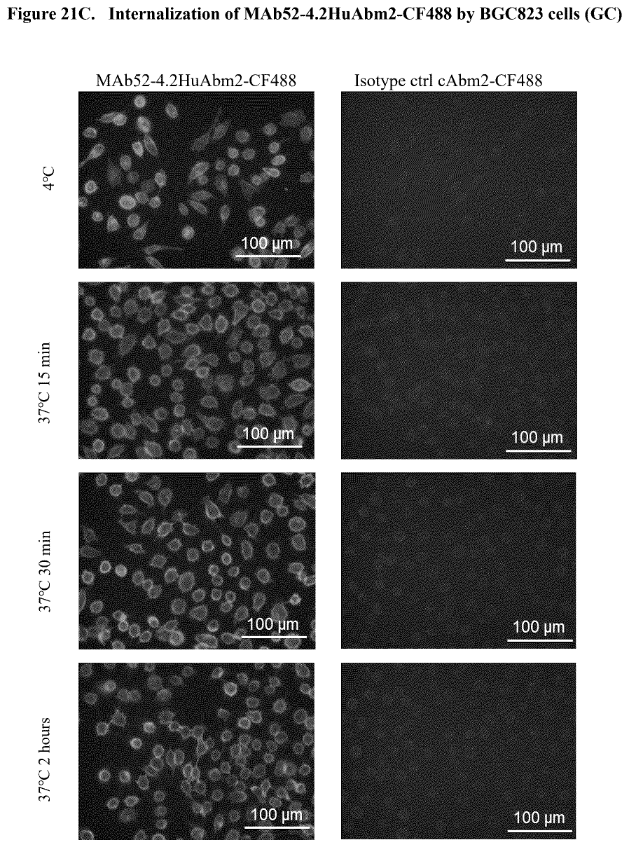

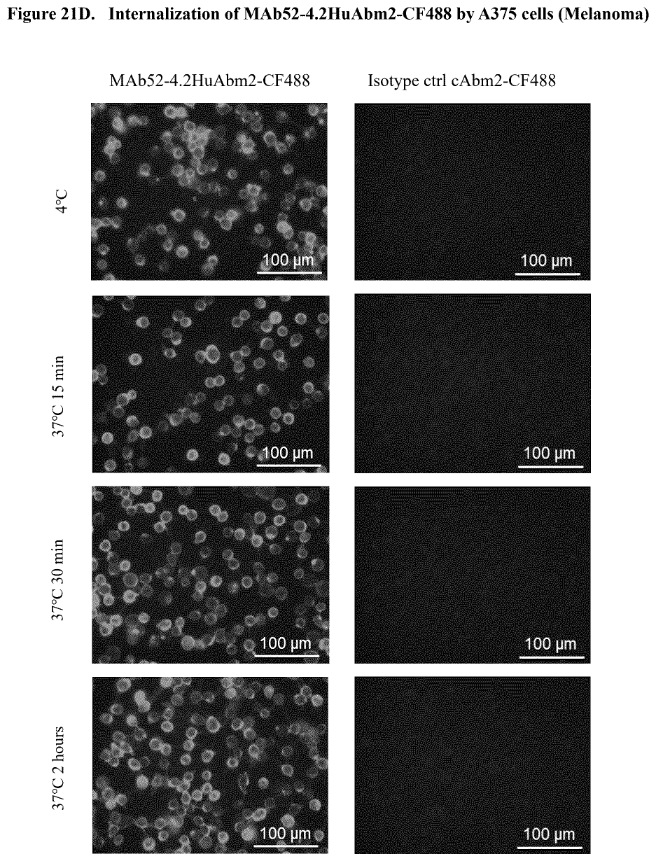

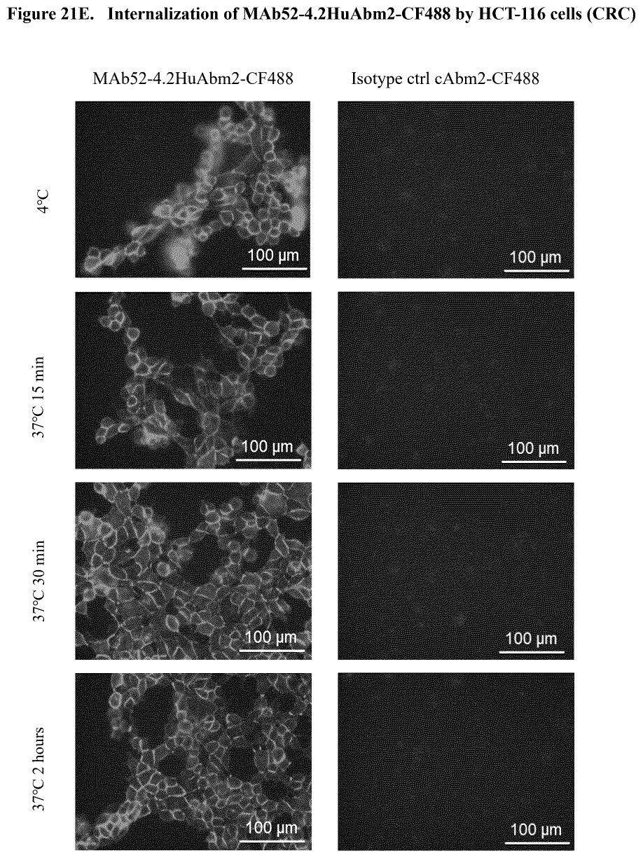

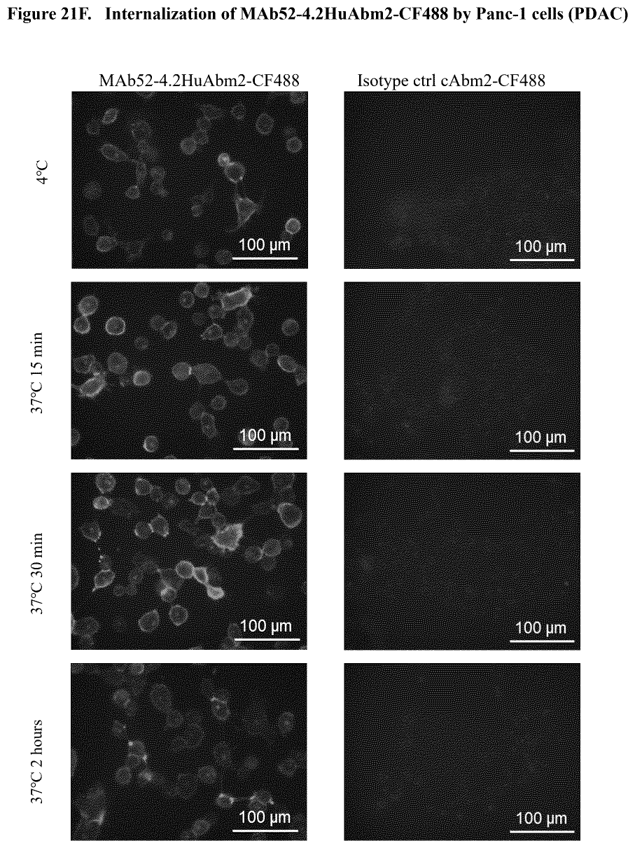

The internalization of MAb52-4.2HuAbm2 was evaluated using the protocol described in Example 3B. CF488-labeled MAb52-4.2HuAbm2 and an isotype control cAbm2 were conjugated with the Mix-n-Stain Antibody Labeling Kit (Biotium, Fremont, CA) following the manufacturer's instructions. Consistent with MAb52-4.2mAb-FITC, A549, BT549d, and BGC823 cells exhibited rapid and robust internalization of MAb52-4.2HuAbm2—CF488 (FIGS. 21A to 21C), whereas HCT-116 cells displayed limited intracellular fluorescence (FIG. 21E). Panc-1 cells showed internalization at 4° C., but no significant increase in intracellular signal was observed after incubation at 37° C. (FIG. 21F). Unlike with MAb52-4.2mAb-FITC, A375 cells did not exhibit efficient internalization of MAb52-4.2HuAbm2 (FIG. 21D). These findings indicate that humanization preserved the internalization capability of MAb52-4.2 mAb, further supporting its potential for ADC development in cancer therapy.

### Example 9

### Preparation and Characterization of MAb52-4.2HuAbm2 ADC

A. Site-Specific Conjugation of MAb52-4.2HuAbm2 with Vc-MMAE

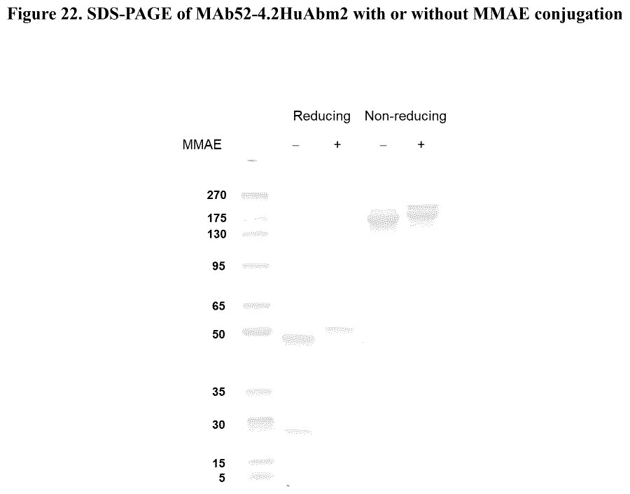

To evaluate whether the ADC derived from MAb52-4.2HuAbm2 has anti-cancer activity like MAb52-4.2cAbm2-MMAE, VcMMAE was used for site-specific conjugation following the procedure described in Example 6. MMAE was conjugated to four free cysteine residues within the heavy chain constant regions of MAb52-4.2HuAbm2, generating an ADC with DAR of approximately 4. SDS-PAGE analysis confirmed successful conjugation, as indicated by the apparent molecular weight shift of both the non-reduced MAb52-4.2HuAbm2-MMAE and the reduced heavy chain-MMAE (FIG. 22).

B. Binding of MMAE-Conjugated MAb52-4.2HuAbm2 and MAb52-4.2cAbm2 with Cancer Cell Lines

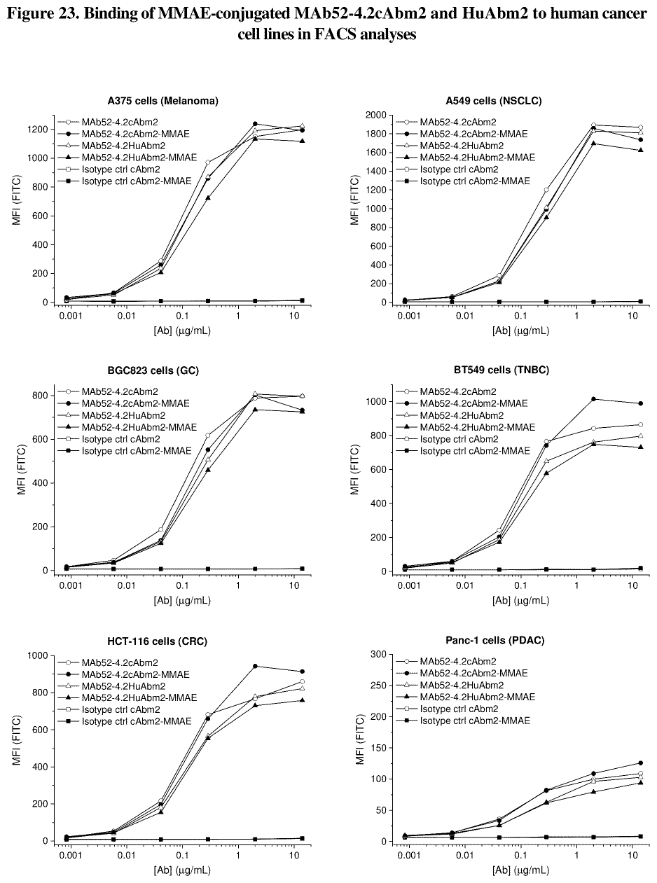

In FACS analyses, both humanized and chimeric MAb52-4.2 antibodies, with or without MMAE-conjugation, exhibited similar binding properties to various cancer cell lines tested (FIG. 23). This suggests that MMAE-conjugation to the Fc regions of the antibodies does not interfere with the binding activity of either MAb52-4.2cAbm2 or MAb52-4.2HuAbm2. Since MAb52-4.2HuAbm2 recognized overlapped epitope like MAb52-4.2cAbm2, and could be efficiently internalized by various cancer cell lines, it is reasonable to assume that MAb52-4.2HuAbm2-MMAE retains the same biological activity as MAb52-4.2cAbm2-MMAE.

### C. In Vitro Inhibitory Effects of MAb52-4.2HuAbm2-MMAE on Cancer Cell Proliferation

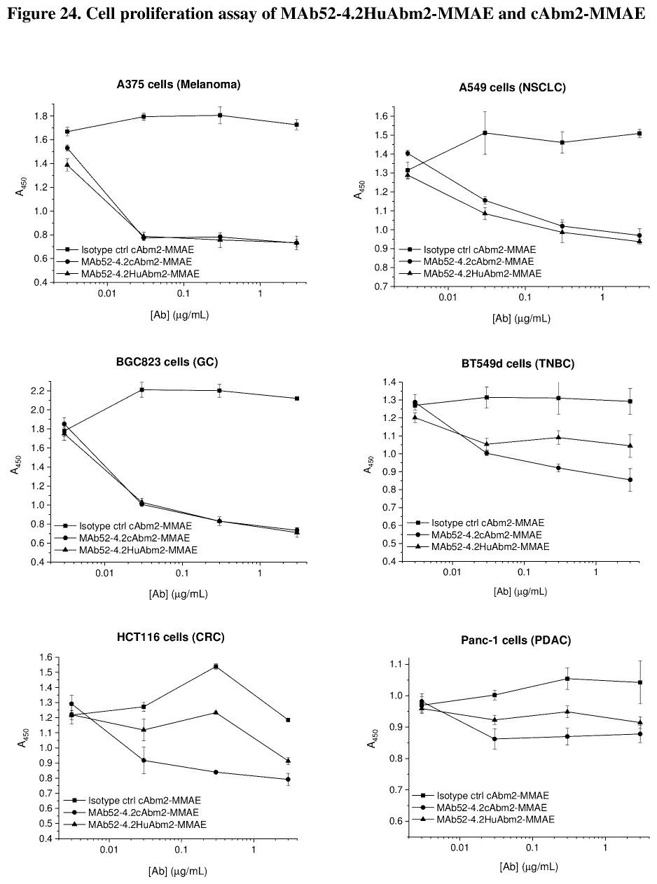

An in vitro cell proliferation assay was conducted following the protocol described in Example 6. MAb52-4.2HuAbm2-MMAE and MAb52-4.2cAbm2-MMAE exhibited similarly potent growth inhibition in A375, A549, and BGC823 cell lines (FIG. 24). However, MAb52-4.2HuAbm2-MMAE appeared to have slightly lower antiproliferative potency than MAb52-4.2cAbm2-MMAE in BT549d and HCT-116 cells, which could be attributed to experimental variations. Both MAb52-4.2HuAbm2— and MAb52-4.2cAbm2-derived ADCs showed minimal effects on Panc-1 cells, aligning with the observed low binding affinity and poor internalization properties with this cell line.

In summary, the humanized MAb52-4.2HuAbm2 retained its binding capability to human CD98hc, whether expressed as a monomer or as a dimeric complex, and various human cancer cell lines. The tested ADC, MAb52-4.2HuAbm2-MMAE, demonstrated sustained inhibitory activity in vitro. These finding suggest that MAb52-4.2HuAbm2 is likely to maintain its anti-cancer potency in vivo, making it a promising drug candidate for cancer therapy in humans.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, “containing”, “having” and “have” are to be read as synonyms and expansively and without limitation. The methods and processes illustratively described herein suitably may be practiced in differing orders of steps, and that they are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1
Sequence Listing

| SEQ ID | | |
|---|---|---|
| NO: | Sequence | Note |
| 1 | CAAATTGTTCTCACCCAGTCTCCAACAATCATGTCTGCATCTCCTGGG | Mouse VL |
| | GAGAAGGTCACCATAGCCTGCAGTGTCAGCTCAAGTGTAAATTACATA | |
| | CACTGGGTCCAGCAGAAGCCAGGCACTTCTCCCAAACTCTGGATTTAT | |
| | AGCACGTCCAAATTGGCTTCTGGAGTCCCTGCTCGCTTCAGTGGCAGT | |
| | GGCTCTGGGACCTCTTATTCTCTCACAGTCAGCCGAATGGAGGCTGAA | |
| | GATGCTGCCACTTATTACTGCCAACAAAGGAGTAGTTACCCGTACACG | |
| | TTCGGAGGGGGGACCAAGCTGGAAATAAGA | |
| 2 | QIVLTQSPTIMSASPGEKVTIACSVSSSVNYIHWVQQKPGTSPKLWIY | Mouse VL |
| | STSKLASGVPARFSGSGSGTSYSLTVSRMEAEDAATYYCQQRSSYPYT | |
| | FGGGTKLEIR | |
| 3 | CAGGTCCAACTGCAGCAGCCTGGGGCTGAACTGGGCAAGCCTGGGACA | Mouse VH |
| | TCAGTGAAGCTGTCCTGCAAGGCTTCTGGCTACACATTCACCAGTTAT | |
| | TGGATGCACTGGGTGAAGCAGAGGCCTGGACAAGGCCTTGAGTGGATT | |
| | GGAAACATTAATCCTAATGGTGGTAAGACTAACTATAATGAGAGGTTC | |
| | AAGAATAAGGCCACACTGACTGTTGACAAATCCTCCAACACAGCCTAC | |
| | ATGCAGCTCACCTCCCTGACATCTGAGGACTCTGCGGTCTACTTCTGT | |
| | ACAAGAGAGATAGACTGGGGCCAAGGCACCACTCTCACAGTCTCCTCA | |
| 4 | QVQLQQPGAELGKPGTSVKLSCKASGYTFTSYWMHWVKQRPGQGLEWI | Mouse VH |
| | GNINPNGGKTNYNERFKNKATLTVDKSSNTAYMQLTSLTSEDSAVYFC | |
| | TREIDWGQGTTLTVSS | |
| 5 | SVSSSVNYIH | CDR-L1 |
| 6 | STSKLAS | CDR-L2 |
| 7 | QQRSSYPYT | CDR-L3 |
| 8 | GYTFTSYWMH | CDR-H1 |
| 9 | NINPNGGKTNYNERFKN | CDR-H2 |
| 10 | EID | CDR-H3 |
| 11 | EIVLTQSPATLSLSPGERATLSCSVSSSVNYIHWYQQKPGQAPRLLIY | HuVL1 |
| | STSKLASGIPARFSGSGSGTDFTLTISSLEPEDFAVYYCQQRSSYPYT | |
| | FGQGTKLEIK | |
| 12 | QVQLVQSGAEVKKPGASVKVSCKASGYTFTSYWMHWVRQAPGQGLEWM | HuVH1 |
| | GNINPNGGKTNYNERFKNRVTMTRDTSTSTVYMELSSLRSEDTAVYYC | |
| | AREIDWGQGTLVTVSS | |
| 13 | EIVLTQSPATLSLSPGERATLACSVSSSVNYIHWVQQKPGQAPRLWIY | HuVL2 |
| | STSKLASGVPARFSGSGSGTDFTLTISSLEPEDFAVYYCQQRSSYPYT | |
| | FGQGTKLEIK | |
| 14 | QVQLVQSGAEVKKPGASVKVSCKASGYTFTSYWMHWVRQAPGQGLEWI | HuVH2 |
| | GNINPNGGKTNYNERFKNRATMTRDTSTSTVYMELSSLRSEDTAVYFC | |
| | TREIDWGQGTLVTVSS | |
| 15 | CCAGAAGGATGATGTCGCTCAG | SLC3A2 Forward |
| | | primer |
| 16 | GAGTAAGGTCCAGAATGACACGG | SLC3A2 Reverse |
| | | primer |
| 17 | GGCGACCTGGAAGTCCAACT | RPLP0 Forward |
| | | primer |
| 18 | CCATCAGCACCACAGCCTTC | RPLP0 Reverse |
| | | primer |
| 19 | CTGCTCACTGGATGGTGGGAAGATGG | Light chain |
| | | reverse primer |
| 20 | AGCTGGGAAGGTGTGCACAC | Heavy chain |
| | | reverse primer |
| 21 | AGCACTAGTGCCGCCACCATGCATTTTCAAGTGCAGAT | VL Cloning |
| | | Forward primer |
| 22 | GAACACGCTGGGCGCCGCATCAGCCCGTCTTATTTC | VL Cloning |
| | | Reverse primer |
| 23 | AGCTCTAGAGCCGCCACCATGGGATGGAACTGTTTCATC | VH Cloning |
| | | Forward primer |
| 24 | GCCTTTGGTGCTAGCTGAGGAGACTGTGAGAGTG | VH Cloning |
| | | Reverse primer |
| 25 | EIVLTQSPATLSLSPGERATLSCRASQSVSSYLAWYQQKPGQAPRLLI | human IGKV3- |
| | YDASNRATGIPARFSGSGSGTDFTLTISSLEPEDFAVYYCQQRSNWP | 11*01 |
| 26 | QVQLVQSGAEVKKPGASVKVSCKASGYTFTSYYMHWVRQAPGQGLEWM | Human IGHV1- |
| | GIINPSGGSTSYAQKFQGRVTMTRDTSTSTVYMELSSLRSEDTAVYYC | 46*01 |
| | AR | |
| 27 | YTFGQGTKLEIK | human IGKJ2 |
| 28 | WGQGTLVTVSS | human IGHJ4 |

### REFERENCES

Bajaj J, Konuma T, Lytle N K, Kwon Hyog Y, Ablack J N, et al. (2016) CD98-mediated adhesive signaling enables the establishment and propagation of acute myelogenous leukemia. Cancer Cell. 30:792-805.
Bianconi D, Fabian E, Herac M, Kieler M, Thaler J, Prager G, et al. (2022) Expression of CD98hc in pancreatic cancer and its role in cancer cell behavior. J Cancer. 13:2271-80.
Bixby D, Wieduwilt M J, Akard L P, Khoury H J, Becker P S, Van E H, et al. (2015) A phase I study of IGN523, a novel anti-CD98 monoclonal antibody in patients with relapsed or refractory acute myeloid leukemia (AML). Blood. 126:3809.
Cantor J M, Ginsberg M H. (2012) CD98 at the crossroads of adaptive immunity and cancer. J Cell Sci. 125:1373-82.
Digomann D, Kurth I, Tyutyunnykova A, Chen O, Löck S, et al. (2019) The CD98 heavy chain is a marker and regulator of head and neck squamous cell carcinoma radiosensitivity. Clin. Cancer Res. 25:3152-3163.
Hasegawa K, Ikeda S, Yaga M, Watanabe K, Urakawa R, et al. (2022) Selective targeting of multiple myeloma cells with a monoclonal antibody recognizing the ubiquitous protein CD98 heavy chain. Sci. Transl. Med. 14: eaax7706.
Hayes G M, Chinn L, Cantor J M, Cairns B, Levashova Z, Tran H, Velilla T, Duey D, Lippincott J, Zachwieja J, Ginsberg M H, van der E H. (2015) Antitumor activity of an anti-CD98 antibody. Int J Cancer. 137:710-20.
Jiang, C., et al. (2020) CD98: A key regulator of immune responses and its therapeutic potential. Nature Reviews Immunology, 20 (4), 215-227.
Junutula J, Raab H, Clark S, et al. (2008). Site-specific conjugation of a cytotoxic drug to an antibody improves the therapeutic index. Nat Biotechnol. 26:925-932.
Kaira K, Oriuchi N, Imai H, Shimizu K, Yanagitani N, Sunaga N, et al. (2009) CD98 expression is associated with poor prognosis in resected non-small-cell lung cancer with lymph node metastases. Ann Surg Oncol. 16:3473-81.
Lim K, Han S H, Han S, Lee J Y, Choi H S, Choi D, Ryu C J. (2024) A monoclonal antibody recognizing CD98 on human embryonic stem cells shows anti-tumor activity in hepatocellular carcinoma xenografts. Cancer Immunol Immunother. 73:231.
Liu, J., et al. (2022) SLC3A2 in metabolic reprogramming and cancer progression: A comprehensive review. Frontiers in Oncology, 12, 1017.
Liu K, Li M, Li Y, Li Y, Chen Z, Tang Y, Yang M, Deng G, Liu H (2024). A review of the clinical efficacy of FDA-approved antibody-drug conjugates in human cancers. Mol Cancer. 23:62.
Livak K J and Schmittgen T D (2001). Analysis of relative gene expression data using real-time quantitative PCR and the 2 (−Delta Delta C(T)) method. Methods. 25:402-8.
Milkereit R, Persaud A, Vanoaica L, Guetg A, Verrey F, Rotin D (2015) LAPTM4b recruits the LAT1-4F2 hc Leu transporter to lysosomes and promotes mTORC1 activation. Nat Commun. 6:7250.
Montero J C, Calvo-Jiménez E, del Carmen S, Abad M, Ocaña A, Pandiella A. (2022) Surfaceome analyses uncover CD98hc as an antibody drug-conjugate target in triple negative breast cancer. J. Exp. Clin. Cancer Res. 41:106.
Nachef M, Ali A K, Almutairi S M, Lee S-H. (2021) Targeting SLC1A5 and SLC3A2/SLC7A5 as a Potential Strategy to Strengthen Anti-Tumor Immunity in the Tumor Microenvironment. Front. Immunol. 12:624324.
Nakao M, Kubo K, Hara A, Hirohashi N, Futagami E, Shichijo S, Sagawa K, Itoh K (1993) A monoclonal antibody (H227) recognizing a new epitope of 4F2 molecular complex associated with T cell activation. Cell Immunol. 152:226-233.
Poettler M, Unseld M, Braemswig K, Haitel A, Zielinski C C, Prager G W. (2013) CD98hc (SLC3A2) drives integrin-dependent renal cancer cell behavior. Mol. Cancer. 12:169.
Prager G W, Feral C C, Kim C, Han J, Ginsberg M H (2007). CD98hc (SLC3A2) interaction with the integrin beta subunit cytoplasmic domain mediates adhesive signaling. J Biol Chem. 282:24477-84.
Rodriguez C F, Escudero-Bravo P, Diaz L, Bartoccioni P, Garcia-Martin C, Gilabert J G, et al. (2021). Structural basis for substrate specificity of heteromeric transporters of neutral amino acids. Proc Natl Acad Sci U.S.A. 118:1-10.
Salter D M, Krajewski A S, Sheehan T, Turner G, Cuthbert R J, Mclean A. (1989) Prognostic significance of activation and differentiation antigen expression in B-cell non-Hodgkin's lymphoma. J. Pathol. 159:211-220.
Xia P and Dubrovska A (2023). CD98 heavy chain as a prognostic biomarker and target for cancer treatment. Front. Oncol. 13:1251100.
Yaga M, Hasegawa K, Ikeda S, Matsubara M, Hiroshima T, et al. (2022) CD98 heavy chain protein is overexpressed in non-small cell lung cancer and is a potential target for CAR T-cell therapy. Sci Rep. 14:17917.
Zhang, X., et al. (2021). Targeting CD98 in cancer therapy: From bench to bedside. Cancer Research, 81 (15), 3892-3901.
Zheng M, (2024) A novel SLC3A2-targeting antibody-drug conjugate exerts potent antitumor efficacy in head and neck squamous cell cancer. Transl Oncol. 45:101981.
Zuchero Y J, Chen X, Bien-Ly N, Bumbaca D, Tong R K, Gao X, Zhang S, Hoyte K, Luk W, Huntley M A, et al. (2016) Discovery of Novel Blood-Brain Barrier Targets to Enhance Brain Uptake of Therapeutic Antibodies. Neuron. 89:70-82.

1. A binding agent of human CD98hc including a light chain variable region wherein CDR 1 to CDR 3 have sequences at least 70% identical to, respectively, SEQ ID NO: 5, SEQ ID NO: 6, and SEQ ID NO: 7.

2. The binding agent of claim 1, wherein CDR 1 to CDR3 of the light chain variable region have sequences identical to, respectively, SEQ ID NO: 5, SEQ ID NO: 6, and SEQ ID NO: 7.

3. A binding agent having a light chain variable region which is at least 70% identical to SEQ ID NO: 2 or SEQ ID NO: 13.

4. A binding agent including a heavy chain variable region wherein CDR 1 to CDR 3 have sequences at least 70% identical to, respectively, SEQ ID NO: 8, SEQ ID NO: 9, and SEQ ID NO: 10.

5. The binding agent of claim 4, wherein CDR1 to CDR3 of the heavy chain variable region have sequences identical to, respectively, SEQ ID NO: 8, SEQ ID NO: 9, and SEQ ID NO: 10.

6. A binding agent having a heavy chain variable region which is at least 70% identical to SEQ ID NO: 4 or SEQ ID NO: 14.

7. An isolated DNA or RNA molecule encoding the light chain variable region of a binding agent of claim 2.

8. An isolated DNA molecule of claim 7 having the nucleotide sequence of SEQ ID NO: 1, or encoding the amino acid sequences in SEQ ID NO: s2 or SEQ ID NO: 13.

9. An isolated DNA or RNA molecule encoding the heavy chain variable region of a binding agent of claim 5.

10. An isolated DNA molecule of claim 9 having the nucleotide sequence of SEQ ID NO: 3, or encoding the amino acid sequences in SEQ ID NO: 4 or SEQ ID NO: 14.

11. The binding agent of claim 1 or 4 wherein the binding agent is a monoclonal antibody.

12. The binding agent of claim 11 wherein the monoclonal antibody is a murine, human, humanized, chimeric, bispecific, or multi-specific antibody.

13. The binding agent of claim 1 wherein the binding agent is a diabody, a single domain antigen binding (SDAB) molecule, or a VL domain.

14. The binding agent of claim 4 wherein the binding agent is a diabody, a single domain antigen binding (SDAB) molecule, a VH domain, or a VHH domain.

15. The binding agent of claim 11 wherein the monoclonal antibody has an IgG1 heavy chain and a κ light chain.

16. The binding agent of claim 1 or 4 wherein the binding agent is a Fab, Fab′, F(ab′) 2, rIgG, Fv, or Fd fragment.

17. The binding agent of claim 1 or 4 wherein the binding agent is a scFv or a sc(Fv)₂.

18. The binding agent of claim 11 wherein the monoclonal antibody is class: IgD, IgE, IgG, IgA, or IgM, or a sub-class of one of said classes.

19. The monoclonal antibody of claim 18 wherein the sub-class is IgG1, IgG2, IgG3, IgG4, IgA1 or IgA2.

20. The monoclonal antibody of claim 18 having a κ light chain.

21. A vector comprising the isolated DNA molecules of claim 8 or 10.

22. The binding agent of claim 1 or 4 conjugated with an anti-tumor agent.

23. The binding agent of claim 22 wherein the anti-tumor agent is monomethyl auristatin E.

23. The binding agent of claim 22 wherein the conjugation is with a linker.

24. The binding agent of claim 23 wherein the linker is cleavable or non-cleavable.

25. The binding agent of claim 24 wherein the linker is a lysosomal-cleavable dipeptide linker.

26. The binding agent of claim 25 wherein the lysosomal-cleavable dipeptide linker includes a para-aminobenzyloxycarbonyl (PABC) spacer and a thiol-reactive maleimidocaproyl group that specifically reacts with a free thiol group in cysteines, as well as anti-tubulin toxin monomethyl auristatin E.


SEQUENCE LISTING (28 sequences)

SEQ ID NO: 1 - DNA, 318 bp, Mus musculus
  source 1..318: mol_type = genomic DNA; note = Mouse VL
caaattgttctcacccagtctccaacaatcatgtctgcatctcctggggagaaggtcaccatagcctgcagtgtcagctcaagtgtaaattacatacactgggtccagcagaagccaggcacttctcccaaactctggatttatagcacgtccaaattggcttctggagtccctgctcgcttcagtggcagtggctctgggacctcttattctctcacagtcagccgaatggaggctgaagatgctgccacttattactgccaacaaaggagtagttacccgtacacgttcggaggggggaccaagctggaaataaga

SEQ ID NO: 2 - protein, 106 aa, Mus musculus
  source 1..106: note = Mouse VL
QIVLTQSPTIMSASPGEKVTIACSVSSSVNYIHWVQQKPGTSPKLWIYSTSKLASGVPARFSGSGSGTSYSLTVSRMEAEDAATYYCQQRSSYPYTFGGGTKLEIR

SEQ ID NO: 3 - DNA, 336 bp, Mus musculus
  source 1..336: mol_type = genomic DNA; note = Mouse VH
caggtccaactgcagcagcctggggctgaactgggcaagcctgggacatcagtgaagctgtcctgcaaggcttctggctacacattcaccagttattggatgcactgggtgaagcagaggcctggacaaggccttgagtggattggaaacattaatcctaatggtggtaagactaactataatgagaggttcaagaataaggccacactgactgttgacaaatcctccaacacagcctacatgcagctcacctccctgacatctgaggactctgcggtctacttctgtacaagagagatagactggggccaaggcaccactctcacagtctcctca

SEQ ID NO: 4 - protein, 112 aa, Mus musculus
  source 1..112: note = Mouse VH
QVQLQQPGAELGKPGTSVKLSCKASGYTFTSYWMHWVKQRPGQGLEWIGNINPNGGKTNYNERFKNKATLTVDKSSNTAYMQLTSLTSEDSAVYFCTREIDWGQGTTLTVSS

SEQ ID NO: 5 - protein, 10 aa, Mus musculus
  source 1..10: note = CDR-L1
SVSSSVNYIH

SEQ ID NO: 6 - protein, 7 aa, Mus musculus
  source 1..7: note = CDR-L2
STSKLAS

SEQ ID NO: 7 - protein, 9 aa, Mus musculus
  source 1..9: note = CDR-L3
QQRSSYPYT

SEQ ID NO: 8 - protein, 10 aa, Mus musculus
  source 1..10: note = CDR-H1
GYTFTSYWMH

SEQ ID NO: 9 - protein, 17 aa, Mus musculus
  source 1..17: note = CDR-H2
NINPNGGKTNYNERFKN

SEQ ID NO: 10 - skipped

SEQ ID NO: 11 - protein, 106 aa, Homo sapiens
  source 1..106: note = HuVL1
EIVLTQSPATLSLSPGERATLSCSVSSSVNYIHWYQQKPGQAPRLLIYSTSKLASGIPARFSGSGSGTDFTLTISSLEPEDFAVYYCQQRSSYPYTFGQGTKLEIK

SEQ ID NO: 12 - protein, 112 aa, Homo sapiens
  source 1..112: note = HuVH1
QVQLVQSGAEVKKPGASVKVSCKASGYTFTSYWMHWVRQAPGQGLEWMGNINPNGGKTNYNERFKNRVTMTRDTSTSTVYMELSSLRSEDTAVYYCAREIDWGQGTLVTVSS

SEQ ID NO: 13 - protein, 106 aa, Homo sapiens
  source 1..106: note = HuVL2
EIVLTQSPATLSLSPGERATLACSVSSSVNYIHWVQQKPGQAPRLWIYSTSKLASGVPARFSGSGSGTDFTLTISSLEPEDFAVYYCQQRSSYPYTFGQGTKLEIK

SEQ ID NO: 14 - protein, 112 aa, Homo sapiens
  source 1..112: note = HuVH2
QVQLVQSGAEVKKPGASVKVSCKASGYTFTSYWMHWVRQAPGQGLEWIGNINPNGGKTNYNERFKNRATMTRDTSTSTVYMELSSLRSEDTAVYFCTREIDWGQGTLVTVSS

SEQ ID NO: 15 - DNA, 22 bp, synthetic construct
  source 1..22: mol_type = other DNA; note = SLC3A2 Forward primer
ccagaaggatgatgtcgctcag

SEQ ID NO: 16 - DNA, 23 bp, synthetic construct
  source 1..23: mol_type = other DNA; note = SLC3A2 Reverse primer
gagtaaggtccagaatgacacgg

SEQ ID NO: 17 - DNA, 20 bp, synthetic construct
  source 1..20: mol_type = other DNA; note = RPLP0 Forward primer
ggcgacctggaagtccaact

SEQ ID NO: 18 - DNA, 20 bp, synthetic construct
  source 1..20: mol_type = other DNA; note = RPLP0 Reverse primer
ccatcagcaccacagccttc

SEQ ID NO: 19 - DNA, 26 bp, synthetic construct
  source 1..26: mol_type = other DNA; note = Light chain reverse primer
ctgctcactggatggtgggaagatgg

SEQ ID NO: 20 - DNA, 20 bp, synthetic construct
  source 1..20: mol_type = other DNA; note = Heavy chain reverse primer
agctgggaaggtgtgcacac

SEQ ID NO: 21 - DNA, 38 bp, synthetic construct
  source 1..38: mol_type = other DNA; note = VL Cloning Forward primer
agcactagtgccgccaccatgcattttcaagtgcagat

SEQ ID NO: 22 - DNA, 36 bp, synthetic construct
  source 1..36: mol_type = other DNA; note = VL Cloning Reverse primer
gaacacgctgggcgccgcatcagcccgtcttatttc

SEQ ID NO: 23 - DNA, 39 bp, synthetic construct
  source 1..39: mol_type = other DNA; note = VH Cloning Forward primer
agctctagagccgccaccatgggatggaactgtttcatc

SEQ ID NO: 24 - DNA, 34 bp, synthetic construct
  source 1..34: mol_type = other DNA; note = VH Cloning Reverse primer
gcctttggtgctagctgaggagactgtgagagtg

SEQ ID NO: 25 - protein, 95 aa, Homo sapiens
  source 1..95: note = human IGKV3-11*01
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 26 - protein, 98 aa, Homo sapiens
  source 1..98: note = Human IGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 27 - protein, 12 aa, Homo sapiens
  source 1..12: note = human IGKJ2
YTFGQGTKLEIK

SEQ ID NO: 28 - protein, 11 aa, Homo sapiens
  source 1..11: note = human IGHJ4
WGQGTLVTVSS